(19) United States
(12) Patent Application Publication
(10) Pub. No.: US 2022/0409747 A1
(43) Pub. Date: Dec. 29, 2022

(54) FIBROBLAST ACTIVATION PROTEIN (FAP)-TARGETED IMAGING AND THERAPY OF CANCERS AND OTHER FIBROTIC AND INFLAMMATORY DISEASES

(71) Applicant: Purdue Research Foundation, West Lafayette, IN (US)

(72) Inventors: Philip Stewart Low, West Lafayette, IN (US); Madduri Srinivasarao, West Lafayette, IN (US); Ramesh Mukkamala, West Lafayette, IN (US)

(21) Appl. No.: 17/761,508

(22) PCT Filed: Sep. 17, 2020
(86) PCT No.: PCT/US2020/051328

§ 371 Date: Mar. 17, 2022

Related U.S. Application Data
(60) Provisional application No. 62/901,792, filed on Sep. 17, 2019.

(51) Int. Cl. A61K 49/10; A61K 47/54; A61K 47/55; A61K 49/08; A61K 49/00; A61K 51/04

(52) U.S. Cl. CPC A61K 49/10; A61K 47/545; A61K 47/55; A61K 49/085; A61K 49/0052; A61K 51/0446

(57) ABSTRACT

Fibroblast activation protein (FAP)-targeting compounds (e.g., conjugates); a method for imaging cancer or fibrosis; and methods for treating an inflammatory disease/disorder and cancer.

### CROSS-REFERENCE TO RELATED APPLICATION

This application claims the benefit of U.S. Provisional Patent Application No. 62/901,792, filed Sep. 17, 2019, which is hereby incorporated by reference in its entirety.

### TECHNICAL FIELD

The present disclosure relates to the development of certain chemical compounds and radicals, such as fibroblast activation protein (FAP-u) targeted ligands. In some instances, the radicals are linked to a drug or an imaging agent. In certain instances, such linked compounds (also referred to herein as “conjugates”) are utilized in various methods, such as the treatment and/or imaging of (e.g., FAP-positive) cancers, fibrotic diseases, and/or inflammatory disorders. In some instances, specifically targeted (e.g., in therapeutic and/or imaging methods) are cancer-associated fibroblasts (CAFs) and/or activated myofibroblasts (e.g., in cancers and/or other fibrotic and inflammatory diseases). In some instances, chemical compounds and/or ligands provided herein have good or improved internalization and residence time in tumor and other diseased sites.

### BACKGROUND

Radio- and chemo-therapies are considered for various cancers, fibrotic disorders, and inflammatory diseases. Often, such therapies are not considered as first line therapies because of adverse (e.g., systemic) effects that can result.

As a result, there is a need for targeted therapies that treat disease (e.g., cancer, fibrotic disease, and/or inflammatory disease) with radio- and/or chemo-therapeutic agents that are suitable for targeting diseased cells and tissue, with minimal or reduced off-target or systemic effects.

In some instances, survival and proliferation of certain tumors is dependent on the percentage of tumor stroma (TSP). A high TSP can be associated with poorer long-term patient survival compared to low TSP (>50% vs. ≤50% respectively). The TSP can also be a significant prognostic factor for tumor relapse, growth, and metastasis.

In certain instances, cancer-associated fibroblasts (CAFs) are abundant in the tumor stroma and perform several important functions to promote tumorigenesis. These functions include, by way of non-limiting example, cytokine secretion and/or extracellular matrix (ECM) production and remodeling. In some instances, such effects result in angiogenesis to promote tumor growth, signaling factors to increase chemoresistance, denser ECM to create an immunosuppressive environment, and enhanced cell motility to direct metastasis. In some instances, such processes parallel the behavior of pathogenic fibroblasts in fibrotic diseases.

In some instances, a prevalent marker of CAFs is fibroblast activation protein alpha (FAPα). Moreover, FAPα is a serine protease (primarily) found on the cell surface of activated fibroblasts in diseased cells and tissue, such as in fibrotic disease, inflammatory disease, and/or cancer (e.g., fibrosis, rheumatoid arthritis, wound healing, and cancer). More than 90% of epithelial carcinomas show FAPα expression in immunohistochemical (IHC) staining. Additional FAPα expression has been found in a subset of primary glioma cell cultures and tumor-associated macrophages (TAMs). However, FAPα expression is very low or nonexistent in the majority of adult tissues. Therefore, because the expression is restricted to the surface of diseased cells, such as carcinomas, FAPα is uniquely qualified as a receptor for selectively delivering pharmacotherapeutics to tumors via ligand-targeting.

### SUMMARY

Provided are compounds (e.g., conjugates) of formula X:

Aₘ-L-B (X)

wherein:
A is a radical of a fibroblast activation protein alpha (FAPα) ligand (targeting moiety) (e.g., with a molecular weight below 10,000);
L is a (e.g., bi-functionalized) linker connecting one or more A groups to B (e.g., through a first covalent bond connecting L to A and a second covalent bond linking L to B);
B is an (e.g., a radical of) imaging agent, a photodynamic therapeutic agent, a radio-imaging agent, a radiotherapeutic agent, a chemotherapeutic agent, an antifibrotic agent, or an anticancer agent (e.g., an anticancer agent that is effective against cancer cells or cancer-associated fibroblasts, myofibroblasts or other tumor microenvironment factors); and
m is 1-6.

Also provided are compounds (e.g., conjugates) of formula I:

A-L-B (I)

wherein
A comprises (e.g., a radical of) a FAPα ligand (e.g., targeting moiety);
L comprises a (e.g., bi-functionalized) linker connecting one or more A groups to B; and
B comprises an (e.g., a radical of) optical imaging agent, a photodynamic therapeutic agent, a radio-imaging agent, a radiotherapeutic agent, a chemotherapeutic agent, an antifibrotic agent, or an anticancer agent (e.g., an anticancer agent that is effective against cancer cells or cancer-associated fibroblasts, myofibroblasts or other tumor microenvironment factors).

Further provided is a method for imaging cancer or fibrosis in a subject with the cancer or the fibrosis, the method comprising administering an effective amount of a compound to a subject in need thereof.

Still further provided is a method for treating an inflammatory disease or disorder, the method comprising administering a therapeutically effective amount of a compound to a subject suffering therefrom.

Even still further provided is a method for treating cancer, the method comprising administering a therapeutically effective amount of a compound to a subject suffering therefrom.

### BRIEF DESCRIPTION OF DRAWINGS

The drawings illustrate generally, by way of example, but not by way of limitation, various embodiments discussed in the present document.

FIG. 1 shows the retrosynthesis of a fibroblast activation protein (FAP) targeted ligand.

FIG. 2 shows the structure of a targeted compound having a target ligand.

FIG. 3 shows increased binding with increasing concentrations (e.g., at 50 nM (A), at 25 nM (B), at 12.5 nM (C), and at 6.25 nM (D)) of a targeting ligand on fibroblast cells having high concentrations of fibroblast activation protein (FAP).

FIG. 4 shows binding of a targeting ligand on fibroblasts having high surface concentrations of FAP at a single concentration after incubation for 1 hour (A), 8 hours (B), 24 hours (C), and 48 hours (D).

FIG. 5 shows binding of a targeting ligand on fibroblasts having high surface concentrations of FAP with at least 100-fold excess of competition ligand (e.g., A: 25 nM targeting ligand, 2.5 μM competitor; B: 25 nM targeting ligand, 5 μM competitor) for 1 hour.

FIG. 6 shows binding (e.g., at 100 nM (A) and at 200 nM (B)) of a targeting ligand on similar fibroblast cells, without high surface concentrations of FAP.

FIG. 7 shows a binding curve for a targeting ligand on fibroblasts with high surface concentrations of FAP (or FAP fibroblasts).

FIG. 8 shows binding curves for a targeting ligand on FAP fibroblasts (targeting ligand only (circles) and targeting ligand and competitor (squares)) and FAP fibroblasts (triangles).

FIG. 9A illustrates imaging results demonstrating in vivo tumor-specific targeting of a targeting ligand after administration to a mammal having a tumor with a FAP-rich environment for 2 hours to 32 hours.

FIG. 9B illustrates imaging results demonstrating in vivo tumor-specific targeting of a targeting ligand after administration to a mammal having a tumor with a FAP-rich environment for 48 hours to 122 hours.

FIG. 9C illustrates the biodistribution of a targeting ligand after administration to a mammal having a tumor with a FAP-rich environment (MDA-MB-231 xenograft mouse) in the tumor, heart, liver, lung, spleen, kidney, intestine, and stomach after 122 hours. Black or white ovals or circles in the images highlight where the targeting ligand is present. Darker shading within the oval or circle represents a higher concentration of targeting ligand than the lighter shading within the oval or circle.

FIG. 10 shows in vivo imaging of a targeting ligand after administration to a mammal having a tumor with a FAP-rich environment (MDA-MB-231 xenograft mouse) after 2 hours and 6 hours both with (right) and without (left) an unlabelled competitor. Black ovals or circles in the images highlight where the targeting ligand is present. Darker shading within the oval or circle represents a higher concentration of targeting ligand than the lighter shading within the oval or circle.

FIG. 11A shows in vivo imaging of a targeting ligand after administration to a mammal having another tumor with a FAP-rich environment (KB xenograft mouse) for 2 hours to 32 hours.

FIG. 11B shows in vivo imaging of a targeting ligand after administration to a mammal having another tumor with a FAP-rich environment (KB xenograft mouse) for 48 hours to 122 hours.

FIG. 11C illustrates the biodistribution of a targeting ligand after administration to a mammal having a tumor with a FAP-rich environment in the tumor, heart, liver, lung, spleen, kidney, intestine, and stomach at 122 hours. Black ovals or circles in the images highlight where the targeting ligand is present. Darker shading within the oval or circle represents a higher concentration of targeting ligand than the lighter shading within the oval or circle.

FIG. 12 shows in vivo imaging of a targeting ligand after administration to a mammal having another tumor with a FAP-rich environment (KB xenograft mouse) after 2 hours and 6 hours both with (right) and without (left) an unlabelled competitor. Black ovals or circles in the images highlight where the targeting ligand is present. Darker shading within the oval or circle represents a higher concentration of targeting ligand than the lighter shading within the oval or circle.

FIG. 13 shows biodistribution of a targeting ligand in the tumor, heart, liver, lung, spleen, kidney, intestine, and stomach at time points 2 h, 4 h, 6 h, 6 h (kidney covered (KC)), 15 h, 24 h, and 122 hours after administration to a mammal having another tumor with a FAP-rich environment (KB xenograft mouse). Black or white arrows, ovals, or circles in the images highlight where the targeting ligand is present. Darker shading adjacent to an arrowhead or within an oval or circle represents a higher concentration of targeting ligand than lighter shading.

FIG. 14A shows in vivo imaging of a targeting ligand after administration to a mammal having another tumor with a FAP-rich environment (FADu xenograft mice M1, M2, M3) 6 hours post-injection.

FIG. 14B illustrates biodistribution of the FAP-targeting compound in the tumor, heart, liver, lung, spleen, kidney, intestine, muscle, and stomach after 6 hours.

FIG. 14C shows in vivo imaging of a competition experiment between an exemplary FAP-targeting compound and an unlabelled competitor 6 hours post-injection to a mammal having another tumor with a FAP-rich environment (FADu xenograft mice M1, M2, and M3).

FIG. 14D illustrates biodistribution of a FAP-targeting compound in the tumor, heart, liver, lung, spleen, kidney, intestine, muscle, and stomach after 6 hours. Black or white arrows, ovals, or circles in the images highlight where the targeting ligand is present. Darker shading adjacent to an arrowhead or within an oval or circle represents a higher concentration of targeting ligand than lighter shading.

FIG. 15A shows in vivo imaging of a targeting ligand after administration to a mammal having another tumor with a FAP-rich environment (HT29 xenograft mice, M1, M2, and M3).

FIG. 15B illustrates the biodistribution of the FAP-targeting compound in the tumor, heart, liver, lung, spleen, kidney, intestine, muscle, and stomach after 6 hours. FIG. 15C shows in vivo imaging of a competition experiment between a targeting ligand and an unlabelled competitor after administration.

FIG. 15C illustrates the biodistribution of the FAP-targeting compound in the tumor, heart, liver, lung, spleen, kidney, intestine, muscle, and stomach after 6 hours. Black or white arrows, ovals, or circles in the images highlight where the targeting ligand is present. Darker shading adjacent to an arrowhead or within an oval or circle represents a higher concentration of targeting ligand than lighter shading.

FIG. 15D. illustrates the biodistribution of the FAP-targeting compound in the tumor, heart, liver, lung, spleen, kidney, intestine, muscle, and stomach after 6 hours. Black or white arrows, ovals, or circles in the images highlight where the targeting ligand is present. Darker shading adjacent to an arrowhead or within an oval or circle represents a higher concentration of targeting ligand than lighter shading in HT29 xenograft mice.

FIG. 16A shows in vivo imaging of a targeting ligand after administration to a mammal having a tumor with a FAP-rich environment (KB tumor xenograft mice (e.g., M1, M2, and M3)).

FIG. 16B illustrates the biodistribution in the tumor, heart, liver, lung, spleen, kidney, intestine, muscle, and stomach.

FIG. 16C shows in vivo imaging of a competition experiment between a targeting ligand and an unlabelled competitor after administration to a mammal having a tumor with a FAP-rich environment (KB tumor xenograft mice (e.g., M1, M2, and M3)).

FIG. 16D illustrates biodistribution in the tumor, heart, liver, lung, spleen, kidney, intestine, muscle, and stomach for the competition study. Black or white arrows, ovals, or circles in the images highlight where the targeting ligand is present. Darker shading adjacent to an arrowhead or within an oval or circle represents a higher concentration of targeting ligand than lighter shading.

FIG. 17A shows in vivo imaging of a targeting ligand after administration to a mammal having a tumor with a FAP-rich environment (MDA-MB-231 tumor xenograft mice (e.g., M1, M2, and M3)).

FIG. 17B illustrates the biodistribution of the FAP-targeting compound in the tumor, heart, liver, lung, spleen, kidney, intestine, muscle, and stomach.

FIG. 17C illustrates the in vivo imaging of a competition experiment between a targeting ligand and an unlabelled competitor 500 nmol.

FIG. 17D illustrates the biodistribution of the FAP-targeting compound in the tumor, heart, liver, lung, spleen, kidney, intestine, muscle, and stomach in the competition study. Black or white arrows, ovals, or circles in the images highlight where the targeting ligand is present. Darker shading adjacent to an arrowhead or within an oval or circle represents a higher concentration of targeting ligand than lighter shading.

FIG. 18A shows in vivo imaging of a targeting ligand after administration to a mammal having another tumor with a FAP-rich environment (U87MG tumor xenograft mice (e.g., M1, M2, and M3)).

FIG. 18B illustrates the biodistribution of the FAP-targeting compound in the tumor, heart, liver, lung, spleen, kidney, intestine, muscle, and stomach.

FIG. 18C shows in vivo imaging of a competition experiment between a targeting ligand and an unlabelled competitor.

FIG. 18D illustrates the biodistribution in the tumor, heart, liver, lung, spleen, kidney, intestine, muscle, and stomach in the competition study. Black or white arrows, ovals, or circles in the images highlight where the targeting ligand is present. Darker shading adjacent to an arrowhead or within an oval or circle represents a higher concentration of targeting ligand than lighter shading.

FIG. 19A shows in vivo imaging of a targeting ligand after administration to a mammal having another tumor with a FAP rich environment (PANC1 tumor xenograft mice (e.g., M1, M2, and M3)).

FIG. 19B illustrates the biodistribution of the a FAP-targeting compound in the tumor, heart, liver, lung, spleen, kidney, intestine, muscle, and stomach.

FIG. 19C shows in vivo imaging of a competition experiment between a targeting ligand provided herein and an unlabelled competitor.

FIG. 19D illustrates the biodistribution in the tumor, heart, liver, lung, spleen, kidney, intestine, muscle, and stomach in the competition study. Black or white arrows, ovals, or circles in the images highlight where the targeting ligand is present. Darker shading adjacent to an arrowhead or within an oval or circle represents a higher concentration of targeting ligand than lighter shading.

FIG. 20A shows in vivo imaging of a targeting ligand after administration to a mammal having another tumor with a FAP-rich environment (4T1 tumor xenograft mice) 2 hours post-injection.

FIG. 20B illustrates the biodistribution of the FAP-targeting compound in the tumor, heart, liver, lung, spleen, kidney, intestine, muscle, and stomach after 6 hours. Black or white arrows, ovals, or circles in the images highlight where the targeting ligand is present. Darker shading adjacent to an arrowhead or within an oval or circle represents a higher concentration of targeting ligand than lighter shading.

FIG. 20C illustrates the biodistribution of the FAP-targeting compound in the tumor, heart, liver, lung, spleen, kidney, intestine, muscle, and stomach after 6 hours. Black or white arrows, ovals, or circles in the images highlight where the targeting ligand is present. Darker shading adjacent to an arrowhead or within an oval or circle represents a higher concentration of targeting ligand than lighter shading in 4T1 xenograft mice.

FIG. 21 shows a displacement binding curve for a targeting ligand in HEK-FAP cells.

FIG. 22 shows displacement binding curves for targeting ligands in HEK-FAP cells.

FIG. 23 shows a Western blot for the level of phosphorylation of Akt (protein kinase B) in transforming growth factor (TGF)-β-stimulated human lung fibroblasts after treatment (e.g., at a concentration of 1 nM, 10 nM, or 100 nM) of a phosphoinositide 3-kinase inhibitor (PI3Ki) or a targeting compound provided herein.

FIG. 24 shows the relative change in expression of collagen 1A1 mRNA in transforming growth factor (TGF)-β-stimulated human lung fibroblasts after the treatment (e.g., at a concentration of 1 nM, 10 nM, or 100 nM) of a phosphoinositide 3-kinase inhibitor (PI3Ki) or a targeting compound provided herein.

### DETAILED DESCRIPTION

### Definitions

Unless defined otherwise, all technical and scientific terms used herein have the same meaning as commonly understood by one of skill in the chemical and biological arts.

Additionally, as used in this specification and the appended claims, the singular forms “a”, “an” and “the” include plural referents unless the content clearly dictates otherwise. Thus, for example, where a compound/composition is substituted with “an” alkyl or aryl, the compound/composition is optionally substituted with at least one alkyl and/or at least one aryl. Furthermore, unless specifically stated otherwise, the term “about” refers to a range of values plus or minus 10% for percentages and plus or minus 1.0 unit for unit values, for example, about 1.0 refers to a range of values from 0.9 to 1.1.

A “therapeutically effective amount” (or “effective amount”) of a compound with respect to use in treatment, refers to an amount of the compound in a preparation which, when administered as part of a desired dosage regimen (to a mammal, such as a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

The term “prophylactic or therapeutic” treatment is art-recognized and includes administration to the patient of one or more compound of the disclosure. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “patient”, “individual” or “subject” refers to a mammal in need of a particular treatment. A patient or subject can be a primate, canine, feline, or equine. A patient or subject can be a bird. The bird can be a domesticated bird, such as chicken. The bird can be a fowl. A patient or subject can be a human.

“Oxo” refers to the ═O radical.

“Alkyl” generally refers to a straight or branched hydrocarbon chain radical consisting solely of carbon and hydrogen atoms, such as having from one to fifteen carbon atoms (e.g., C₁-C₁₅ alkyl). Disclosures provided herein of an “alkyl” are intended to include independent recitations of a saturated “alkyl,” unless otherwise stated. An alkyl can comprise one to thirteen carbon atoms (e.g., C₁-C₁₃ alkyl). An alkyl can comprise one to eight carbon atoms (e.g., C₁-C₈ alkyl). An alkyl can comprise one to five carbon atoms (e.g., C₁-C₅ alkyl). An alkyl can comprise one to four carbon atoms (e.g., C₁-C₄ alkyl). An alkyl can comprise one to three carbon atoms (e.g., C₁-C₃ alkyl). An alkyl can comprise one to two carbon atoms (e.g., C₁-C₂ alkyl). An alkyl can comprise one carbon atom (e.g., C₁ alkyl). An alkyl can comprise five to fifteen carbon atoms (e.g., C₅-C₁₅ alkyl). An alkyl can comprise five to eight carbon atoms (e.g., C₅-C₈ alkyl). An alkyl can comprise two to five carbon atoms (e.g., C₂-C₅alkyl). An alkyl can comprise three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond.

“Alkoxy” refers to a radical bonded through an oxygen atom of the formula —O-alkyl, where alkyl is an alkyl chain as defined above.

“Alkylene” or “alkylene chain” generally refers to a straight or branched divalent alkyl group linking the rest of the molecule to a radical group, such as having from one to twelve carbon atoms, for example, methylene, ethylene, propylene, i-propylene, n-butylene, and the like.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene.

“Aralkyl” or “aryl-alkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain.

“Carbocyclyl” or “cycloalkyl” refers to a stable non-aromatic monocyclic or polycyclic hydrocarbon radical consisting solely of carbon and hydrogen atoms, which includes fused or bridged ring systems, having from three to fifteen carbon atoms.

A carbocyclyl can comprise three to ten carbon atoms. A carbocyclyl can comprise five to seven carbon atoms. The carbocyclyl is attached to the rest of the molecule by a single bond. Carbocyclyl or cycloalkyl is saturated (i.e., containing single C—C bonds only) or unsaturated (i.e., containing one or more double bonds or triple bonds). Examples of saturated cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like.

“Carbocyclylalkyl” refers to a radical of the formula —R^{c}-carbocyclyl where R^{c} is an alkylene chain as defined above.

“Halo” or “halogen” refers to bromo, chloro, fluoro or iodo substituents.

“Haloalkyl” refers to an alkyl radical, as defined above, that is substituted by one or more halogen radicals, as defined above, for example, trifluoromethyl, difluoromethyl, fluoromethyl, 2,2,2-trifluoroethyl, 1-fluoromethyl-2-fluoroethyl, and the like.

The term “heteroalkyl” refers to an alkyl group as defined above in which one or more skeletal carbon atoms of the alkyl are substituted with a heteroatom (with the appropriate number of substituents or valencies—for example, —CH₂— may be replaced with —NH— or —O—). For example, each substituted carbon atom is independently substituted with a heteroatom, such as wherein the carbon is substituted with a nitrogen, oxygen, selenium, or other suitable heteroatom. In some instances, each substituted carbon atom is independently substituted for an oxygen, nitrogen (e.g. —NH—, —N(alkyl)-, or —N(aryl)- or having another substituent contemplated herein), or sulfur (e.g. —S—, —S(═O)—, or —S(═O)₂—). A heteroalkyl is attached to the rest of the molecule at a carbon atom of the heteroalkyl. A heteroalkyl is attached to the rest of the molecule at a heteroatom of the heteroalkyl. A heteroalkyl is a C₁-C₁₈ heteroalkyl. A heteroalkyl is a C₁-C₁₂ heteroalkyl. A heteroalkyl is a C₁-C₆ heteroalkyl. A heteroalkyl is a C₁-C₄ heteroalkyl. Heteroalkyl can include alkoxy, alkoxyalkyl, alkylamino, alkylaminoalkyl, aminoalkyl, heterocycloalkyl, heterocycloalkyl, and heterocycloalkylalkyl, as defined herein.

“Heteroalkylene” refers to a divalent heteroalkyl group defined above which links one part of the molecule to another part of the molecule.

“Heterocyclyl” refers to a stable 3- to 18-membered non-aromatic ring radical that can comprise two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which optionally includes aromatic, fused, and/or bridged ring systems. The heteroatoms in the heterocyclyl radical are optionally oxidized. The heterocyclyl radical is partially or fully saturated. Disclosures provided herein of an “heterocyclyl” are intended to include independent recitations of heterocyclyl comprising aromatic and non-aromatic ring structures, unless otherwise stated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, 1,3-benzodioxolyl, 1,4-benzodioxanyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, indolinyl, isoindolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. Examples of such N-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“Heteroaryl” refers to a radical derived from a 3- to 18-membered aromatic ring radical that can comprise two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, benzofuranyl, benzooxazolyl, benzo[d]thiazolyl, benzothiadiazolyl, benzo[b][1,4]dioxepinyl, benzo[b][1,4]oxazinyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl).

The compounds disclosed herein can contain one or more asymmetric centers and thus give rise to enantiomers, diastereomers, and other stereoisomeric forms that are defined, in terms of absolute stereochemistry, as (R)- or (S)—. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

As used herein, the term “linker” generally refers to a portion of a compound that forms a chemical bond with an A (e.g., a binding ligand) and/or B (e.g., a therapeutic agent or a imaging agent). In particular, a “linker” can connect two or more functional parts of a molecule to form a compound provided herein. Illustratively, the linker may comprise atoms selected from C, N, O, S, Si, and P; C, N, O, S, and P; or C, N, O, and S. The linker may connect different functional capabilities of the compound, such as the FAP ligand and the PI3K inhibitor. The linker may comprise a several linker groups, such as, for example, in the range from about 2 to about 100 atoms in the contiguous backbone. The linker can be a releasable linker. The linker can be a non-releasable linker.

A compound can be a monovalent conjugate (e.g., a compound comprising one binding ligand (as described elsewhere herein, e.g., one FAP-binding ligand)). A compound can be a bivalent conjugate (e.g., a compound comprising one or more binding ligand (as described elsewhere herein, e.g., one or more FAP-binding ligand) conjugated to a therapeutic agent or an imaging agent (e.g., through a linker) (as described elsewhere herein)). A compound can be a multivalent conjugate (e.g., a compound comprising two or more binding ligands (as described elsewhere herein, e.g., two or more FAP-binding ligands) conjugated to a multipoint linker).

The binding ligand (also referred to herein as the targeting ligand or the targeting moiety) can be a compound (or radical thereof) that binds to a biological molecule (e.g., a polypeptide (e.g., an enzyme)) localized to a particular cell, tissue, organ, or the like. The binding ligand can be a fibroblast activation protein (FAP) ligand (or a radical thereof). The binding ligand can be a fibroblast activation protein alpha (FAPα) ligand (or a radical thereof).

The therapeutic agent (or a radical thereof) can be any entity that can produce a desirable physiological response. The therapeutic agent (or a radical of) can be an antifibrotic agent, an anticancer agent, a chemotherapeutic agent, a radiotherapeutic agent, or the like. A therapeutic agent can be a compound (e.g., or a radical thereof) that is effective against (e.g., effective at eliminating, destroying, reducing (e.g., reducing the amount of), or lessening the effects of) cancer cells or pro-fibrotic cells (e.g., cancer-associated fibroblasts, myofibroblasts, or the like (e.g., other tumor microenvironment factors)). Examples of a therapeutic agent (or a radical thereof) include, but are not limited to, a photodynamic therapeutic agent, a radiotherapeutic agent, a chemotherapeutic agent, an antifibrotic agent, and an anti-cancer agent. The therapeutic agent provided herein can be a phosphoinositide-3-kinase (PI3K) inhibitor (or a radical thereof). The therapeutic agent can be an anti-cancer agent (or a radical thereof). The therapeutic agent can be an anti-fibrotic agent (or a radical thereof). The therapeutic agent can be a compound (or a radical thereof) selected from a tumor growth factor (TGF) R/Smad inhibitor, a Wnt/0-catenin inhibitor, a kinase inhibitor (e.g., a kinase inhibitor for Vascular Endothelial Growth Factor Receptor (VEGFR), a kinase inhibitor for Fibroblast Growth Factor Receptors (FGFR), a kinase inhibitor for platelet-derived growth factor receptor (PDGFR), a kinase inhibitor for focal adhesion kinase (FAK), or a kinase inhibitor for Rho-associated protein kinase (ROCK)), a toll-like receptor agonist (TLR), a nuclear factor kappa-light-chain-enhancer of activated B cells (NF-κB) inhibitor, an inhibitor of collagen synthesis, and a phosphoinositide-3-kinase (PI3K) inhibitor. The therapeutic agent can be a phosphoinositide-3-kinase (PI3K) inhibitor (or a radical thereof).

The imaging agent can be a compound (or a radical thereof) that emits a detectable signal (e.g., an electromagnetic signal (e.g., a radio signal, a fluorescent signal, gamma rays) or a mass). Examples of an imaging agent include, but are not limited to, a radio-imaging agent (e.g., a PET imaging agent or a SPECT imaging agent), a fluorescent imaging agent (e.g., a fluorescent dye), or the like.

### Compounds

Provided are compounds (e.g., conjugates) of formula X:

Aₘ-L-B (X)

wherein
A is a radical of a fibroblast activation protein alpha (FAPα) ligand (targeting moiety) (e.g., with a molecular weight below 10,000);
L is a (e.g., bi-functionalized) linker connecting one or more A groups to B (e.g., through a first covalent bond connecting L to A and a second covalent bond linking L to B);
B is an (e.g., a radical of) optical imaging agent, a photodynamic therapeutic agent, a radio-imaging agent, a radiotherapeutic agent, a chemotherapeutic agent, an antifibrotic agent, or an anticancer agent (e.g., an anticancer agent that is effective against cancer cells or cancer-associated fibroblasts, myofibroblasts or other tumor microenvironment factors); and

m is 1-6.

A can be a radical of FAPα ligand with a molecular weight below less than 10,000, 7,500 less than 5,000, less than 2,500, less than 1,000, less than 760, less then 500; from about 500 to about 10,000 g/mol, about 1,000 to about 7,500 g/mol, about 750 g/mol to about 1,500 g/mol, about 1000, to about 5,000 g/mol or about 500 to about 2,500 g/mol.

m can be 1. m can be 2. m can be 3. m can be 4. m can be 5. m can be 6. m can be 1 to 3, 2 to 4, 1 to 5.

The disclosure also relates to compounds (e.g., conjugates) of formula I:

A-L-B (I)

wherein:
A comprises (e.g., a radical of) a FAPα ligand (e.g., targeting moiety);
L comprises a (e.g., bi-functionalized) linker connecting one or more A groups to B; and
B comprises (e.g., a radical of) an optical imaging agent, a photodynamic therapeutic agent, a radio-imaging agent, a radiotherapeutic agent, a chemotherapeutic agent, an antifibrotic agent, or an anticancer agent (e.g., an anticancer agent that is effective against cancer cells or cancer-associated fibroblasts, myofibroblasts or other tumor microenvironment factors).

The targeting moiety can bind to an activated fibroblast expressing FAPα and such activated fibroblast is involved in cancer or inflammatory diseases. The targeting moiety can have a molecular weight below 10,000. L can comprise a bi-functionalized linker. The (e.g., biofunctionalized) linker can form a chemical bond with A and B. L can be a (e.g., bi-functionalized) linker connecting one or more A groups to B (e.g., through a first covalent bond connecting L to A and a second covalent bond linking L to B). B can comprise (e.g., a radical of) an imaging agent, a radio-imaging agent, a photodynamic therapeutic agent, a chemotherapeutic agent, an antifibrotic agent and/or a radiotherapeutic agent, wherein B is an anticancer agent that is effective against cancer cells or cancer-associated fibroblasts, myofibroblasts, or other tumor microenvironment factor.

A can have a structure represented by the formula I-A:

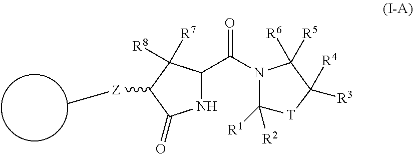

wherein

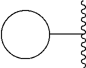

is a functionalized 5- to 10-membered N-containing aromatic or non-aromatic mono- or bicyclic heterocycle, said heterocycle optionally further comprising 1 to 3 heteroatoms selected from oxygen, nitrogen, and sulfur;
Z is a bond, substituted or unsubstituted alkylene (e.g., —CH₂—), substituted or unsubstituted amino (e.g., —NH—), —O—, or —S—;
T is substituted or unsubstituted methylene (—CH₂—), substituted or unsubstituted amino (—NH—), —O—, or —S—;
R¹ and R² are each independently selected from the group consisting of —H, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, —SO₂F and 5-tetrazolyl;
R³ and R⁴ are each independently selected from the group consisting of —H, —OH, F, Cl, Br, I, —C₁₋₆alkyl, —O—C₁₋₆alkyl, and —S—C₁₋₆alkyl;
R⁵, R⁶, R⁷, and R⁸ are each independently selected from group consisting of H, alkyl and halo; and

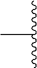

is a point of attachment of the FAPα binding ligand (e.g., through the Linker, L, or the imaging/therapeutic agent moiety, B), wherein the point of attachment can be through any of the carbon atoms of the 5- to 10-membered N-containing aromatic or non-aromatic mono- or bicyclic heterocycle or 1 °, 2° amines or with functionalized alkyl or cycloalkyl motif, as well as stereoisomers and pharmaceutically acceptable salts thereof.

A can have a structure represented by the formula I-B:

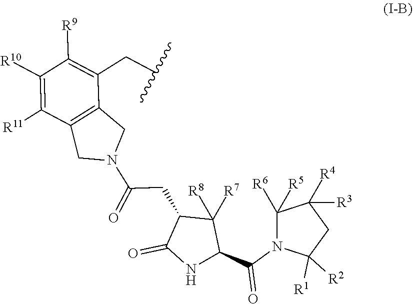

T is substituted or unsubstituted methylene (—CH₂—), substituted or unsubstituted amino (—NH—), —O—, or —S—;
R¹ and R² are each independently selected from the group consisting of —H, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, —SO₂F and 5-tetrazolyl;
R³ and R⁴ are each independently selected from the group consisting of —H, —OH, F, Cl, Br, I, —C₁₋₆alkyl, —O—C₁₋₆alkyl, and —S—C₁₋₆alkyl;
R⁵, R⁶, R⁷, and R⁸ are each independently selected from group consisting of H, alkyl and halo; and
R⁹, R¹⁰, and R¹¹ are each independently selected from group consisting of H, —C₁₋₆alkyl, —O—C₁₋₆alkyl, —S—C₁₋₆ alkyl, F, Cl, Br and I.

A can have a structure represented by the formula I-C:

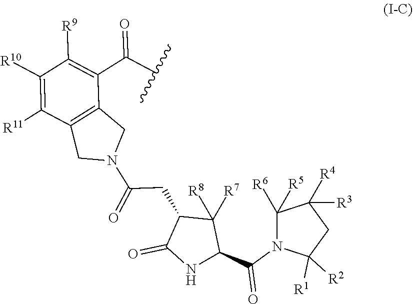

T is substituted or unsubstituted methylene (—CH₂—), substituted or unsubstituted amino (—NH—), —O—, or —S—;
R¹ and R² are each independently selected from the group consisting of —H, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, —SO₂F, and 5-tetrazolyl;
R³ and R⁴ are each independently selected from the group consisting of —H, —OH, F, Cl, Br, I, —C₁₋₆alkyl, —O—C₁₋₆alkyl, and —S—C₁₋₆alkyl;
R⁵, R⁶, R⁷, and R⁸ are each independently selected from group consisting of H, alkyl and halo; and
R⁹, R¹⁰, and R¹¹ are each independently selected from group consisting of H, —C₁₋₆alkyl, —O—C₁₋₆alkyl, —S—C₁₋₆ alkyl, F, Cl, Br and I.

A can have a structure represented by the following formulae:

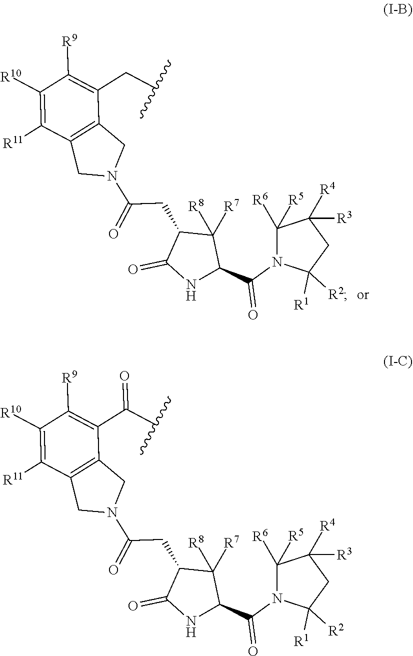

wherein T is substituted or unsubstituted methylene (—CH₂—), substituted or unsubstituted amino (—NH—), —O—, or —S—;
R¹ and R² are each independently selected from the group consisting of —H, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, —SO₂F, and 5-tetrazolyl;
R³ and R⁴ are each independently selected from the group consisting of —H, —OH, F, Cl, Br, I, —C₁₋₆alkyl, —O—C₁₋₆alkyl, and —S—C₁₋₆alkyl;
R⁵, R⁶, R⁷, and R⁸ are each independently selected from group consisting of H, alkyl and halo; and
R⁹, R¹⁰, and R¹¹ are each independently selected from group consisting of H, —C₁₋₆alkyl, —O—C₁₋₆alkyl, —S—C₁₋₆ alkyl, F, Cl, Br and I.

A can have a structure represented by the formula X-A:

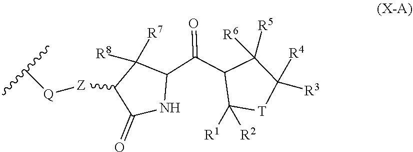

wherein:
Q is aryl, heteroaryl, or heterocyclyl (e.g., comprising aryl and non-aryl ring structures) (e.g., 5- to 10-membered N-containing aromatic or non-aromatic mono- or bicyclic heterocycle, said heterocycle optionally further comprising 1 to 3 heteroatoms selected from O, N, and S);
Z is a bond, substituted or unsubstituted C₁-C₃ alkylene (e.g., —CH₂—), substituted or unsubstituted heteroalkyl (e.g., 1-3 atoms in length), amino (e.g., NH), —O—, or —S—;
T is substituted or unsubstituted methylene (—CH₂—), substituted or unsubstituted amino (—NH—), —O—, or —S— (e.g., wherein the substitution of T is C₁-C₃ alkyl, haloalkyl, or halo);
R¹ and R² are each independently selected from the group consisting of —H, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, —SO₂F, —CONH₂, and 5-tetrazolyl;
R³ and R⁴ are each independently selected from the group consisting of —H, —OH, F, Cl, Br, I, —C₁₋₆alkyl, —O—C₁₋₆alkyl, and —S—C₁₋₆alkyl; and
R⁵, R⁶, R⁷, and R⁸ are each independently selected from group consisting of H, alkyl, and halo.

Q can be attached to L (e.g., L or L₁). Q can be aryl, heteroaryl, or heterocyclyl. The heterocyclyl can comprise aryl and non-aryl ring structures. Q can be attached to L at a heteroalkyl, an alkyl, or an aryl position of Q. Q can be attached to L at an aryl position of Q. Q can be attached to L via a nitrogen atom (e.g., of L). Q can be attached to L via a triazolyl or an amide (e.g., of L). The heteroaryl can comprise aryl and non-aryl ring structures. The heteroaryl or the heterocyclyl can comprise 1 to 3 heteroatoms selected from O, N, and S. The heterocyclyl can comprise 1 to 3 heteroatoms selected from O, N, and S. Q can be a 5- to 10-membered N-containing aromatic or non-aromatic mono- or bicyclic heterocycle (e.g., optionally comprising aryl and non-aryl ring structures). Q can be a N-attached heterocyclyl (e.g., optionally comprising aryl and non-aryl ring structures). Q can be a C₆-C₉—N-attached heterocyclyl (e.g., optionally comprising aryl and non-aryl ring structures). The N-attached heterocyclyl is attached to Z via a N-heterocycloalkyl. Q can be (e.g., an N-attached) isoindolinyl (e.g., wherein the N is attached to Z).

Z can be a bond, substituted or unsubstituted C₁-C₃ alkylene, substituted or unsubstituted heteroalkylene (e.g., 1-3 atoms in length), amino (e.g., NH), —O—, or —S—. Z can be a bond. Z can be substituted methylene. Z can be —CH₂—. Z can be substituted ethylene. Z can be ethylene substituted with oxo. Z can be —C(CO)CH₂—. Z can be —CH₂CH₂—. Z can be a C₁-C₃ heteroalkylene.

A can have a structure represented by the formula X-B:

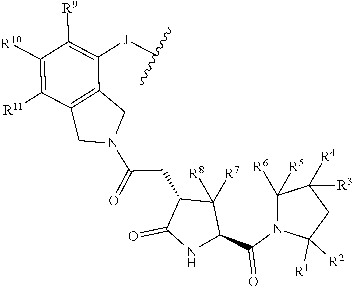

wherein
Q is aryl, heteroaryl, or heterocyclyl (e.g., comprising aryl and non-aryl ring structures); (e.g., 5- to 10-membered N-containing aromatic or non-aromatic mono- or bicyclic heterocycle, said heterocycle optionally further comprising 1 to 3 heteroatoms selected from O, N, and S),
T is substituted or unsubstituted methylene (—CH₂—), substituted or unsubstituted amino (—NH—), —O—, or —S— (e.g., wherein the substitution of T is C₁-C₃ alkyl, haloalkyl, or halo);
J is C(R^{J})₂, wherein each R^{J} is independently H or alkyl, or both R^{J} are taken together to form oxo;
R¹ and R² are each independently selected from the group consisting of —H, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, —SO₂F, —CONH₂, and 5-tetrazolyl;
R³ and R⁴ are each independently selected from the group consisting of —H, —OH, F, Cl, Br, I, —C₁₋₆alkyl, —O—C₁₋₆alkyl, and —S—C₁₋₆alkyl;
R⁵, R⁶, R⁷, and R⁸ are each independently selected from group consisting of H, alkyl, and halo; and
R⁹, R¹⁰, and R¹¹ are each independently selected from group consisting of H, —C₁₋₆alkyl, —C₁₋₆haloalkyl, —O—C₁₋₆alkyl, —S—C₁₋₆ alkyl, F, Cl, Br and I.

J can be attached to L (e.g., L or L₁). J can be attached to L via a nitrogen atom. J can be attached to L via a triazolyl or an amide (e.g., of L). J can be C(R^{J})₂, wherein each R^{J} is independently H or alkyl, or both R^{J} are taken together to form oxo. J can be C₁-C₃alkyl. J can be —CH₂—. J can be —CH₂CH₂—. J can be C═O.

T can be substituted or unsubstituted methylene (e.g., —CH₂—), substituted or unsubstituted amino (e.g., —NH—), —O—, or —S—. The substitution of T can be C₁-C₃ alkyl, C₁-C₃ haloalkyl, or (for the methylene) halo. T can be (—CH₂—). The substitution of T can be C₁-C₃ alkyl, haloalkyl, or halo. T can be unsubstituted.

R¹ and R² are each independently selected from the group consisting of —H, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, —SO₂F, —CONH₂, and 5-tetrazolyl. R¹ and R² can each be independently selected from the group consisting of H, —CN, —CHO, and —B(OH)₂. R¹ and R² can each be independently selected from the group consisting of H, —CN, —CHO, and —CONH₂. R¹ can be H. R² can be —CN, —CHO, —B(OH)₂, or —CONH₂. R¹ can be H and R² can be —CN, —CHO, —B(OH)₂, or —CONH₂. R¹ can be H and R² can be —CN. R¹ can be H and R² can be —CHO. R¹ can be H and R² can be —B(OH)₂. R¹ can be H and R² can be —CONH₂.

R³ and R⁴ can each be independently selected from the group consisting of —H, —OH, F, Cl, Br, I, —C₁₋₆alkyl, —O—C₁₋₆alkyl, and —S—C₁₋₆alkyl. R³ and R⁴ can each be independently —H or —F. R³ can be H and R⁴ can be —F. R³ can be F and R⁴ can be —F.

R¹ can be H, R² can be —CN, R³ can be H and R⁴ can be —F. R¹ can be H, R² can be —CN, R³ can be F and R⁴ can be —F. R¹ can be H, R² can be —CHO, R³ can be H and R⁴ can be —F. R¹ can be H, R² can be —CHO, R³ can be F and R⁴ can be —F. R¹ can be H, R² can be —B(OH)₂, R³ can be H and R⁴ can be —F. R¹ can be H, R² can be —B(OH)₂, R³ can be F and R⁴ can be —F. R¹ can be H, R² can be —CONH₂, R³ can be H and R⁴ can be —F. R¹ can be H, R² can be —CONH₂, R³ can be F and R⁴ can be —F.

R⁵, R⁶, R⁷, and R⁸ can each be independently selected from group consisting of H, alkyl, and halo. R⁵, R⁶, R⁷, and R⁸ can each be H.

R⁹, R¹⁰, and R¹¹ can each be independently selected from group consisting of H, —C₁₋₆alkyl, —C₁₋₆haloalkyl, —O—C₁₋₆alkyl, —S—C₁₋₆ alkyl, F, Cl, Br and I. R⁹, R¹⁰, and R¹¹ can each be independently selected from group consisting of H, —C₁₋₆haloalkyl, F, and Cl. R⁹ and R¹¹ can be H and R¹⁰ can be H, —C₁₋₆haloalkyl, F, or Cl. R⁹ and R¹¹ can be H and R¹⁰ can be H, —CF₃, F, or Cl. R⁹ and R¹¹ can be H and R¹⁰ can be —CF₃. R⁹ and R¹¹ can be H and R¹⁰ can be F. R⁹ and R¹¹ can be H and R¹⁰ can be C₁. R⁹, R¹⁰, and R¹¹ can be H.

A can be attached to L via a nitrogen atom (e.g., of L). A can be attached to L via a triazolyl or an amide (e.g., of L).

A can be selected from the group consisting of:

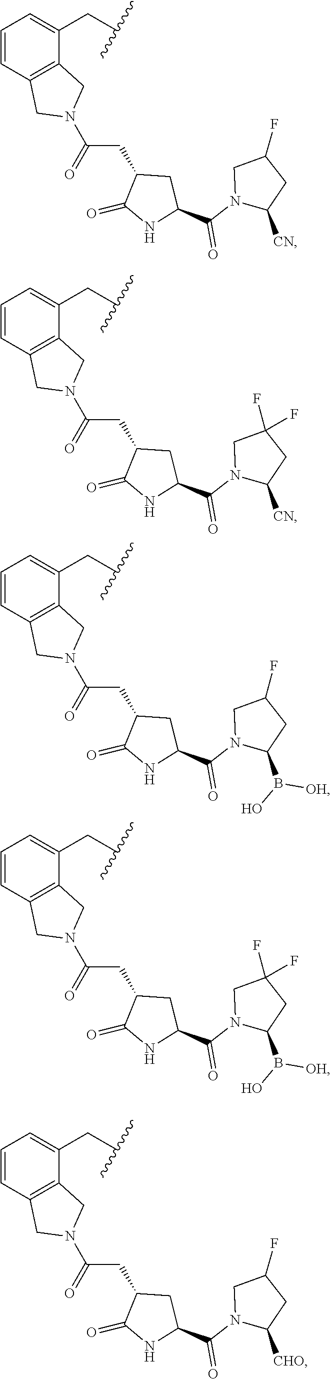 [SMILES: *Cc1cccc2c1CN(C(=O)C[C@@H]1C[C@@H](C(=O)N3CC(F)C[C@H]3C#N)NC1=O)C2 ; *Cc1cccc2c1CN(C(=O)C[C@@H]1C[C@@H](C(=O)N3CC(F)(F)C[C@H]3C#N)NC1=O)C2 ; *Cc1cccc2c1CN(C(=O)C[C@@H]1C[C@@H](C(=O)N3CC(F)C[C@H]3B(O)O)NC1=O)C2 ; *Cc1cccc2c1CN(C(=O)C[C@@H]1C[C@@H](C(=O)N3CC(F)(F)C[C@H]3B(O)O)NC1=O)C2 ; *Cc1cccc2c1CN(C(=O)C[C@@H]1C[C@@H](C(=O)N3CC(F)C[C@H]3C=O)NC1=O)C2]

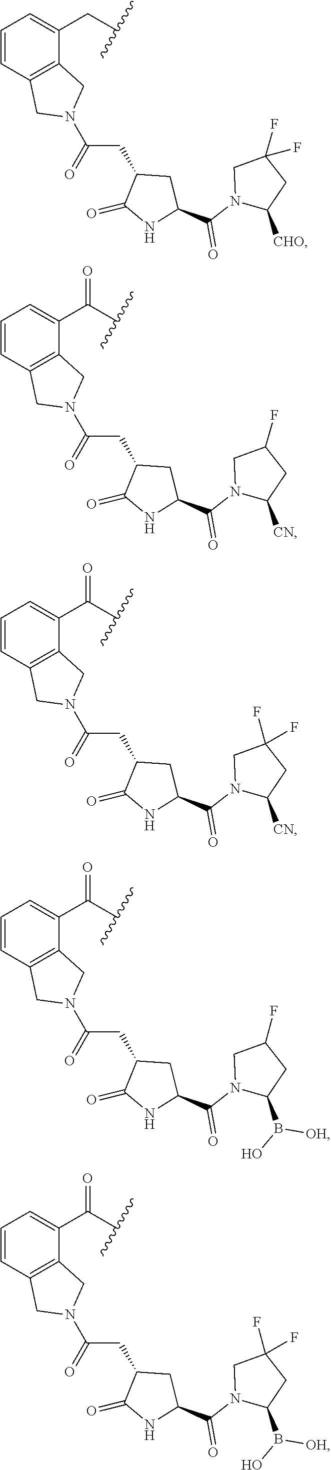 [SMILES: *Cc1cccc2c1CN(C(=O)C[C@@H]1C[C@@H](C(=O)N3CC(F)(F)C[C@H]3C=O)NC1=O)C2 ; *C(=O)c1cccc2c1CN(C(=O)C[C@@H]1C[C@@H](C(=O)N3CC(F)C[C@H]3C#N)NC1=O)C2 ; *C(=O)c1cccc2c1CN(C(=O)C[C@@H]1C[C@@H](C(=O)N3CC(F)(F)C[C@H]3C#N)NC1=O)C2 ; *C(=O)c1cccc2c1CN(C(=O)C[C@@H]1C[C@@H](C(=O)N3CC(F)C[C@H]3B(O)O)NC1=O)C2 ; *C(=O)c1cccc2c1CN(C(=O)C[C@@H]1C[C@@H](C(=O)N3CC(F)(F)C[C@H]3B(O)O)NC1=O)C2]

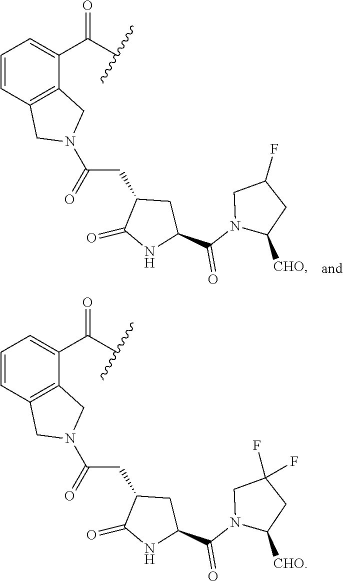 [SMILES: *C(=O)c1cccc2c1CN(C(=O)C[C@@H]1C[C@@H](C(=O)N3CC(F)C[C@H]3C=O)NC1=O)C2 ; *C(=O)c1cccc2c1CN(C(=O)C[C@@H]1C[C@@H](C(=O)N3CC(F)(F)C[C@H]3C=O)NC1=O)C2]

A can be selected from the group consisting of:

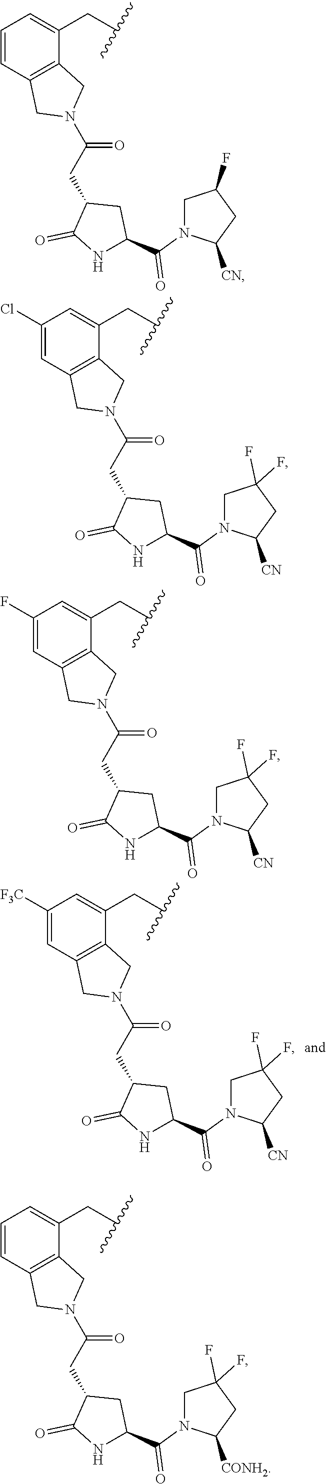

A (e.g., a FAPα binding ligand) can have a binding affinity to a FAP (e.g., FAPα) in the range between about 1 nM to about 25 nM, such as 1 nM to about 25 nM or about 1 nM to 25 nM.

L can be a linker, such as any suitable linker. L can be a non-releasable linker. L can be a releasable linker.

L can comprise one or more linker groups, each linker group independently selected from the group consisting of alkyl(ene), heteroalkyl(ene), heterocycloalkyl(ene), heteroaryl, aryl, alkoxy, thioether, disulfide, carboxylic acid, anhydride, carbonate, carbamate, thioether, sugar, and peptide. L can comprise one or more linker groups, each linker group independently selected from the group consisting of polyethylene glycol (PEG), alkyl(ene), disulfide, amide, carboxylic acid, anhydride, carbonate, ester, carbamate, thioether, triazole, sugar, and peptide. L can comprise one or more linker group, each linker group independently selected from the group consisting of polyethylene glycol (PEG), alkyl(ene), disulfide, amide, carboxylic acid, carbonate, ester, phenyl, triazole, and carbamate. L can comprise one or more linker groups, each linker group independently selected from the group consisting of polyethylene glycol (PEG), alkyl(ene), disulfide, amide, carboxylic acid, phenyl, triazole, ester, and carbonate. L can comprise one or more linker groups, each linker group independently selected from the group consisting of polyethylene glycol (PEG), alkyl(ene), disulfide, amide, carboxylic acid, ester, and carbonate. L can comprise one or more linker groups, each linker group independently selected from the group consisting of polyethylene glycol (PEG), alkyl(ene), disulfide, and amide. L can comprise one or more linker groups, each linker group independently selected from the group consisting of alkyl(ene), disulfide, and amide. L can comprise one or more linker groups, each linker group independently selected from the group consisting of amide, alkyl(ene), PEG, phenyl, and triazole. L can comprise one or more linker groups, each linker group independently selected from the group consisting of PEG, alkyl(ene), and amide. L can comprise one or more linker groups, each linker group independently selected from the group consisting of alkyl(ene) and amide. L can comprise one or more linker groups, each linker group independently selected from the group consisting of PEG and amide. The linker can comprise one or more triazole linker groups. The linker can comprise one or more disulfide linker groups. The linker can comprise one or more amide linker groups. The linker can comprise one or more PEG linker groups.

L can comprise one or more releasable groups.

L can be (covalently) attached to A via an amide linker group. L can be (covalently) attached to B via an amide linker group. L can be independently (covalently) attached to A and B via amide linker groups.

L can be (covalently) attached to A via a triazole linker group. L can be (covalently) attached to B via a triazole linker group. L can be independently (covalently) attached to A and B via triazole linker groups.

L can be (covalently) attached to A via a triazole linker group. L can be (covalently) attached to B via an amide linker group. L can be attached to A via a triazole linker group and attached to B via an amide linker group.

L can be (covalently) attached to A via an amide linker group. L can be (covalently) attached to B via a carbamate linker group. L can be attached to A via an amide linker group and attached to B via a carbamate linker group.

The linker can be a bivalent linker (e.g., connecting a single A to a single B).

The linker can be a multivalent linker (e.g., connecting two or more A to a single B). The linker can be a releasable linker. The linker can be a non-releasable linker.

L can be (L¹)ₒ-Y-(L₂)ₚ, wherein:
each L¹ is a first linker;
each L² is a second linker;
Y is a third linker;
is an integer from 1-5; and
p is an inter from 1-5.

L¹ and L² can be the same. L¹ and L² can be different. Each L¹ can be connected to an A group (and the Y group). Each L² can be connected to a B group (and the Y group). o and m can be the same, such as 1-6, 1-3, or 1. p can be 1. o can be 1. p and o can each be 1.

Each L¹ and L² independently comprise an oligoethylene glycol (chain), a polyethylene glycol (chain), an alkyl (chain), an oligopeptide (chain), or a polypeptide (chain). Each L¹ and L² independently comprise an oligoethylene glycol (chain) or a polyethylene glycol (chain).

Each L¹ and L² independently comprise a triazole or an amide.

Each L¹ and L² independently comprise an oligopeptide (chain) or a polypeptide (chain). Each L¹ and L² independently comprise a peptidoglycan (chain).

Each L¹ and L² independently comprises an oligoproline or an oligopiperidine.

Each L¹ and L² can be independently a length from 15-200 angstroms (Å).

o can be an integer from 1-5. o can be an integer from 1-3. o can be 1.

p can be an integer from 1-5. p can be an integer from 1-3. p can be 1.

Also provided is a multivalent conjugate having the formula II:

(A-S)ₘY-L-B

wherein:
A is a radical of a FAPα ligand (targeting moiety) (e.g., with a molecular weight below 10,000);
S is a spacer (e.g., having a length for the arms of the multivalent targeting ligand (e.g., drug) to reach multiple adjacent FAPs on a target cell);
Y is a linker;
L is a (e.g., bi-functionalized) linker connecting one or more A groups to B (e.g., through a first covalent bond connecting L to A and a second covalent bond linking L to B); and
B is a radical of a fluorescent dye, a photodynamic therapeutic agent, a radio-imaging agent, a radiotherapeutic agent, a chemotherapeutic agent, an antifibrotic agent, or an anticancer agent (e.g., an anticancer agent that is effective against cancer cells or cancer-associated fibroblasts, myofibroblasts or other tumor microenvironment factor); and
m is 2-6.

The spacer can be the optimal length for the arms of the multivalent drug to reach to multiple adjacent FAPs on a target (e.g., cancer or pro-fibrotic) cell.

S can comprise an oligoethylene, a polyethyleneglycol, an alkyl chain, an oligopeptide or a polypeptide. S can be an oligoethylene glycol or a polyethylene glycol.

S can be an oligopeptide or polypeptide.

S can be a peptidoglycan.

The spacer can be a rigid linker. S can be a rigid linker, such as, for example, an oligoproline or an oligopiperidine.

S can have a length of at least 15 angstroms (Å). S can have a length of at most 200 angstroms (Å). S can have a length from 15-200 angstroms (Å).

Y can be a linker that connects multiple arms of the compound (e.g., conjugate). Y can have a repeating structure. Y can comprise a releasable bond. L can comprise a disulfide bond. Y can comprise at least one citric acid group (or a radical thereof). Y can comprise one or more triazole. Y can comprise one or more amine. Y can comprise one or more amide. Y can have an aromatic core (e.g., an aryl core or a heteroaryl core). Y can have an alkyl(ene) core. Y can have an amine core. Y can be N(L¹)₃ (e.g., wherein L¹ can be as described elsewhere herein). Y can be phenyl substituted with three L¹ (e.g., wherein L¹ as described elsewhere herein). Y can be C(L¹)₄ (e.g., wherein L¹ can be as described elsewhere herein).

Y can be attached to a single L¹. Y can be attached to a single L². Y can be attached to a single L¹ and a single L². Y can be independently connected to each L¹ and L² by an amide bond. Y can be attached to L.

Y can be a linker (e.g., a multivalent linker) that connects multiple arms of the compound (e.g., conjugate). Y can have a repeating structure. Y can comprise at least one citric acid group (or a radical thereof). The linker can have the following structure:

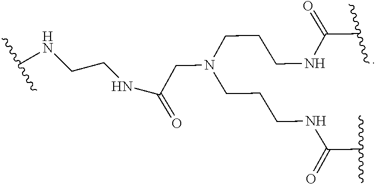 [SMILES: *NCCNC(=O)CN(CCCNC(*)=O)CCCNC(*)=O]

Y can be a linker (e.g., a multivalent linker) that connects multiple arms of the compound (e.g., conjugate) and can comprise a linker (e.g., a repeating unit) of the following structure:

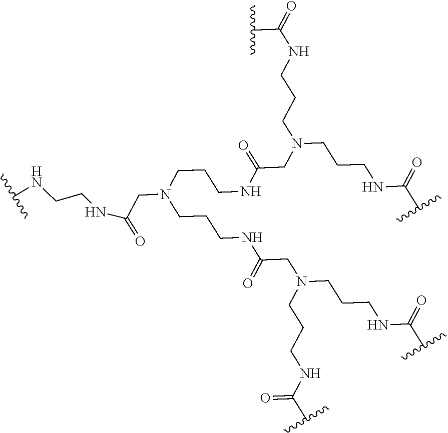

Y can be a linker that connects multiple arms of the compound (e.g., conjugate) that can have a citric acid-based linker. Y can be a linker (e.g., a multivalent template) that connects multiple arms of the compound (e.g., conjugate) and can have a (e.g., citric acid-based) linker of the following structure:

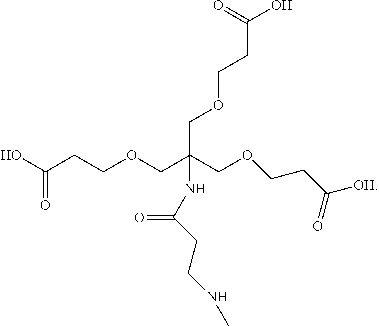 [SMILES: CNCCC(=O)NC(COCCC(=O)O)(COCCC(=O)O)COCCC(=O)O]

Y can be a linker (e.g., a multivalent linker) that connects multiple arms of the compound (e.g., conjugate) and can have a (e.g., citric acid-based) linker of the following structure:

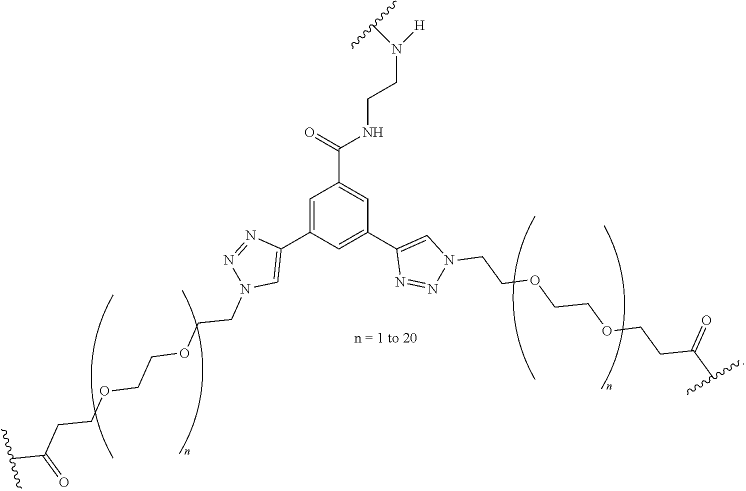

Y can be a linker (e.g., a multivalent linker) that connects multiple arms of the compound (e.g., conjugate) and can have a (e.g., citric acid-based) linker of the following structure:

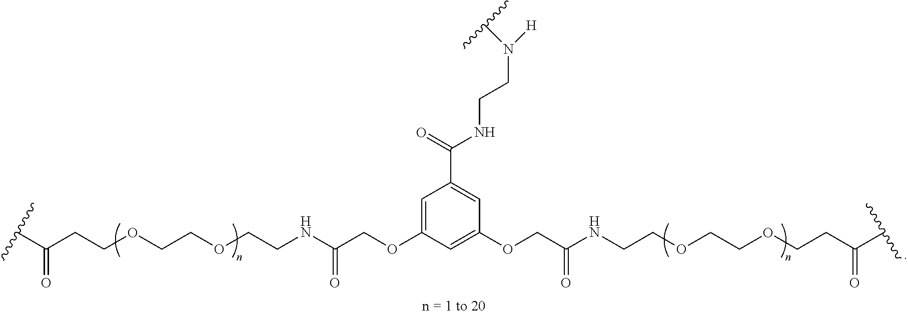

L can comprise at least one linker group, each linker group selected from the group consisting of polyethylene glycol (PEG), alkyl, sugar, and peptide. The linker can be a polyethylene glycol- (PEG-) (e.g., pegylated-), alkyl-, sugar-, and peptide-based dual linker.

L can be a non-releasable linker (e.g., bivalently (e.g., covalently) attached to B and A). L can be a releasable linker (e.g., bivalently (e.g., covalently) attached to B and A).

L, L1, L2, or any combination thereof can comprise one or more linker groups having the following structure:

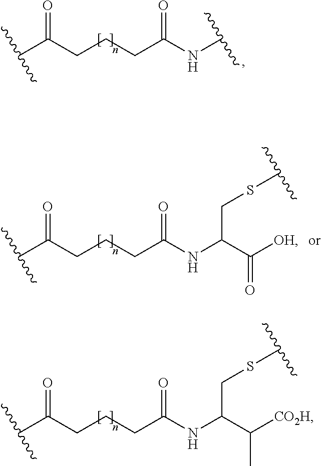

wherein n is 0 to 10.

L can comprise one or more linker groups having the following structure:

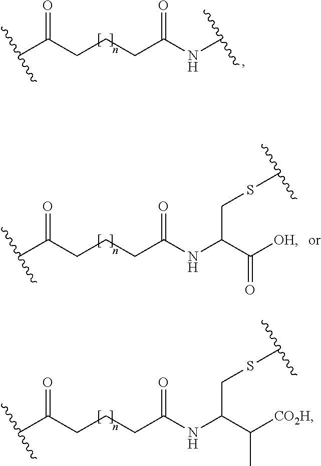

wherein n is 0 to 10.

L can comprise one or more linker groups having the following structure:

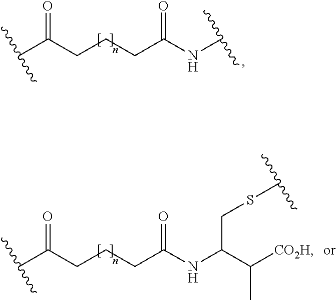

wherein n is 0 to 10.

L can comprise one or more linker groups having the following structure:

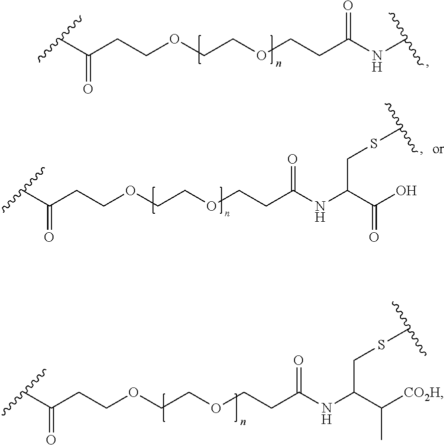

wherein n is 1 to 32.

L can comprise one or more linker groups having the following structure:

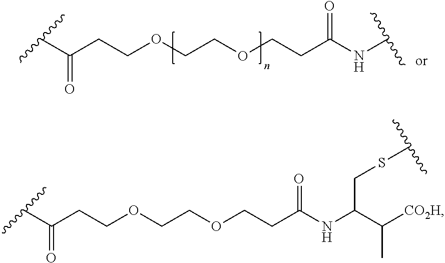

wherein n is 1 to 32.

L can comprise one or more linker groups having the following structure:

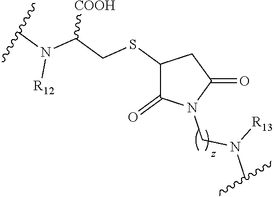

wherein:
R₁₂ and R₁₃ can each be independently H or C₁-C₆ alkyl; and
z is an integer from 1 to 8.

L can comprise one or more linker groups having the following structure:

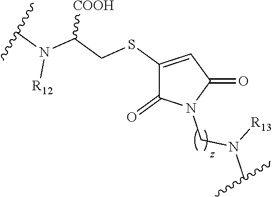

wherein:
R₁₂ and R₁₃ can each be independently H or C₁-C₆ alkyl; and
z is an integer from 1 to 8.

L, L¹, L², or any combination thereof can comprise one or more linker groups having the following structure:

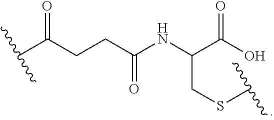 [SMILES: *SCC(NC(=O)CCC(*)=O)C(=O)O]

L can comprise one or more linker groups having the following structure:

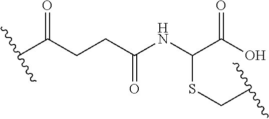 [SMILES: *CSC(NC(=O)CCC(*)=O)C(=O)O]

L can comprise one or more linker groups having the following structure:

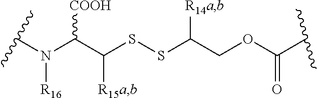

wherein:
R₁₆ is H or C₁-C₆ alkyl; and
R₁₄ₐ, R_{14b}, and R₁₅ₐ, R_{15b} can each be independently H or C₁-C₆ alkyl.

L can have the following structure:

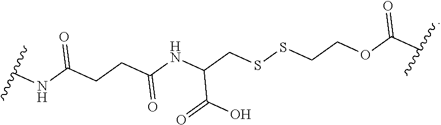 [SMILES: *NC(=O)CCC(=O)NC(CSSCCOC(*)=O)C(=O)O]

L can comprise one or more linker groups having the following structure:

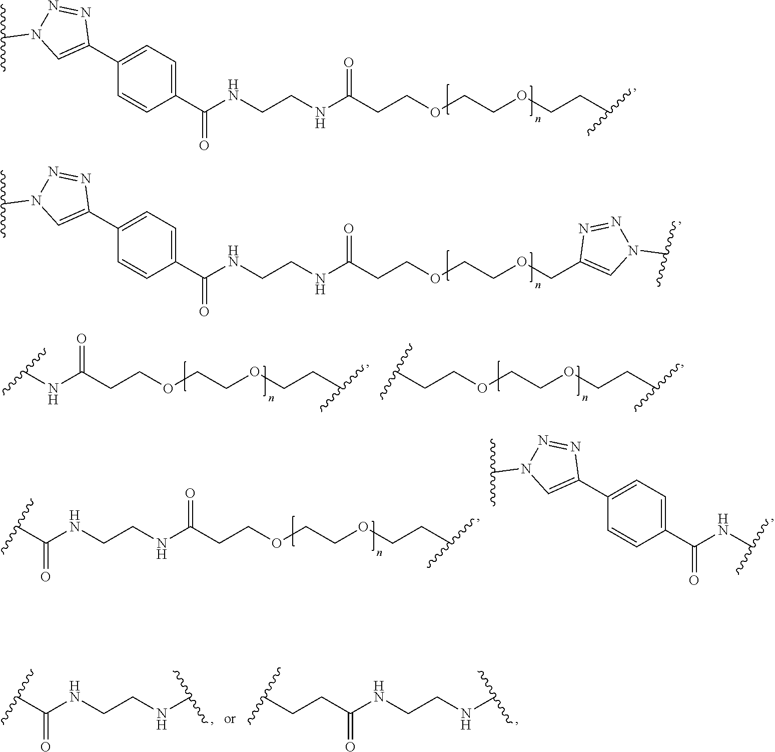

wherein n is 0 to 15.

B can be attached to L via a carbon atom or a nitrogen atom (e.g., of L). B can be attached to L via a triazolyl. B can be attached to L via an oxo (e.g., an ester). B can be attached to L via an amide (e.g., of L).

B can be an optical dye (or a radical thereof). B can be a fluorescent dye (or a radical thereof) (e.g., useful for fluorescence guided surgery (FGS)). The fluorescent dye can comprise a fluorescent dye group, each fluorescent dye group selected from the group consisting of a carbocyanine, an indocarbocyanine, an oxacarbocyanine, a thiacarbocyanine, a merocyanine, a polymethine, acoumarine, a rhodamine, a xanthene, a fluorescein, and the like. The fluorescent dye (or radical thereof) can be borondipyrromethane (BODIPY), CyS, CyS.S, Cy7, VivoTag-680, VivoTag-S680, VivoTag-S7S0, AlexaFluor660, AlexaFluor680, AlexaFluor700, AlexaFluor7S0, 10 AlexaFluor790, Dy677, Dy676, Dy682, Dy7S2, Dy780, DyLightS47, Dylight647, HiLyte Fluor 647, HiLyte Fluor 680, HiLyte Fluor 7S0, IRDye 800CW, IRDye 800RS, IRDye 700DX, ADS780WS, ADS830WS, ADS832WS, S0456, or the like.

The payload can have an excitation from 600 nanometers (nm) to 1000 nm. The payload can have an emission from 700 nm to 1800 nm.

B can be a fluorescent dye group (or a radical thereof) having the following structure:

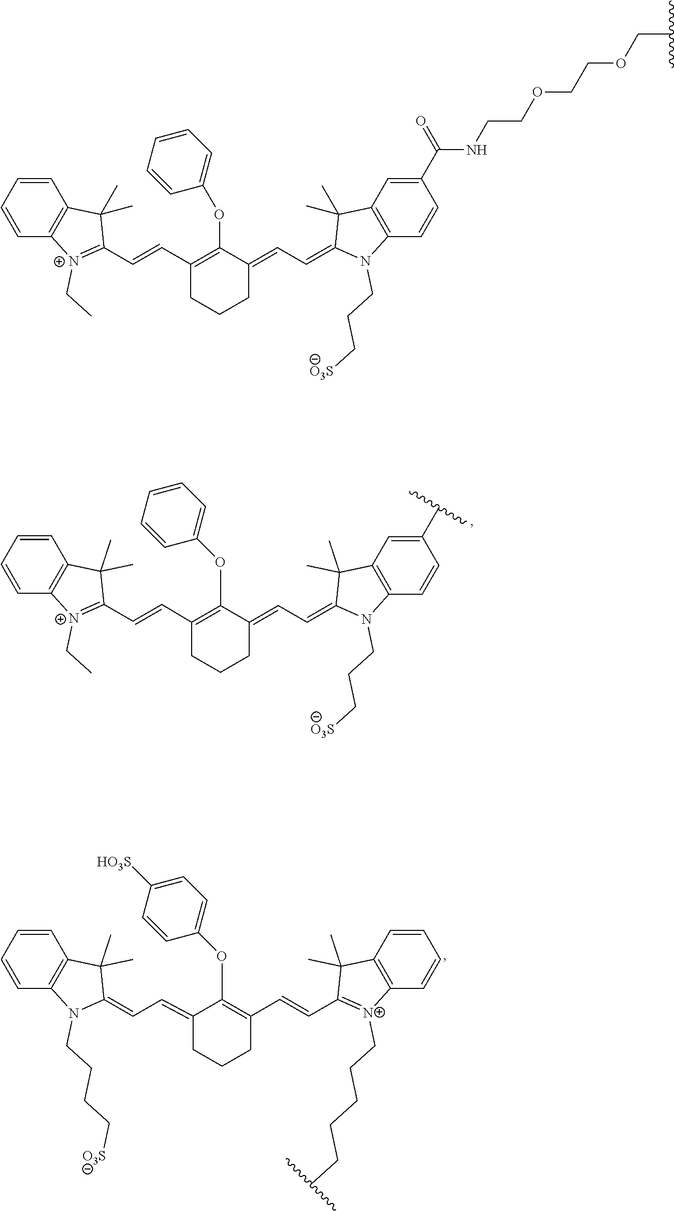

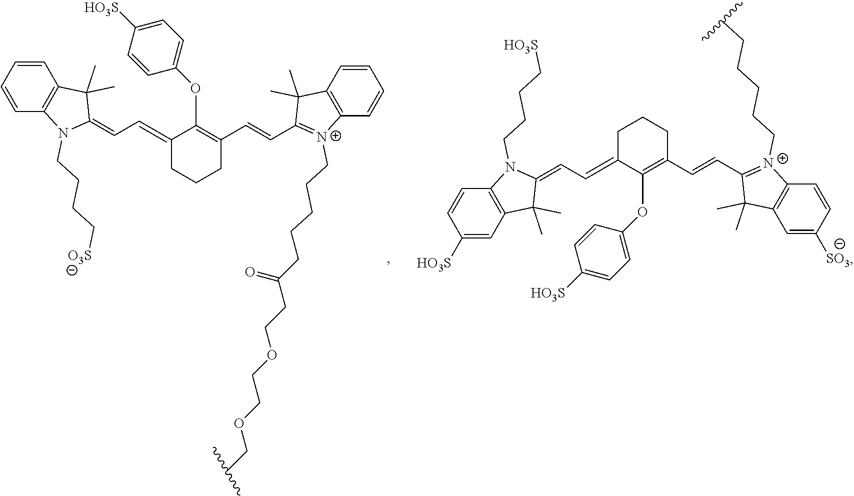

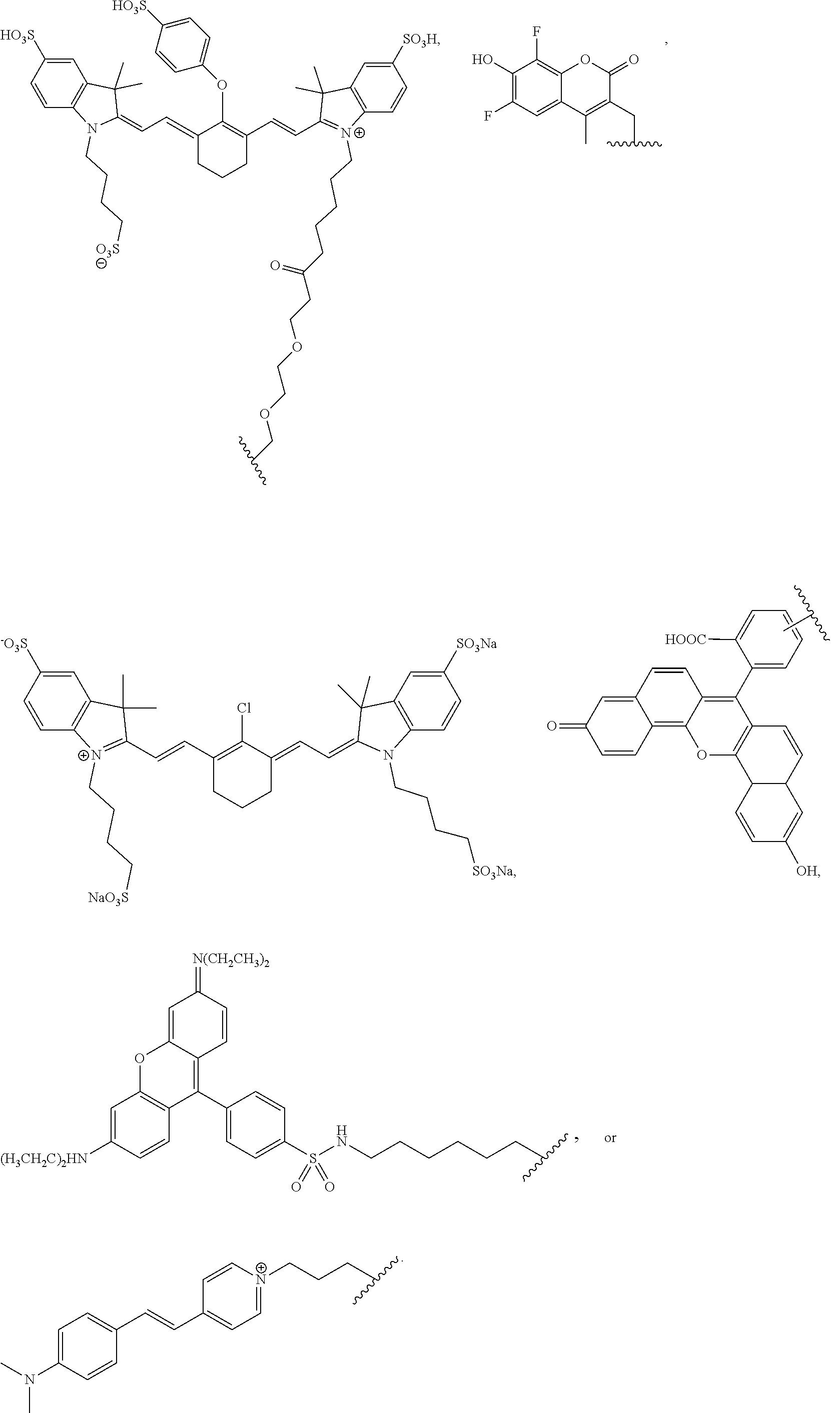

The compound can be a FAP-targeted ligand (or a radical thereof) attached to a linker comprising one or more linker group, each linker group selected from alkyl, pegylated, and peptidoglycan, wherein the linker can further be attached to a fluorescent dye described herein.

A-L-B can have the following structure:

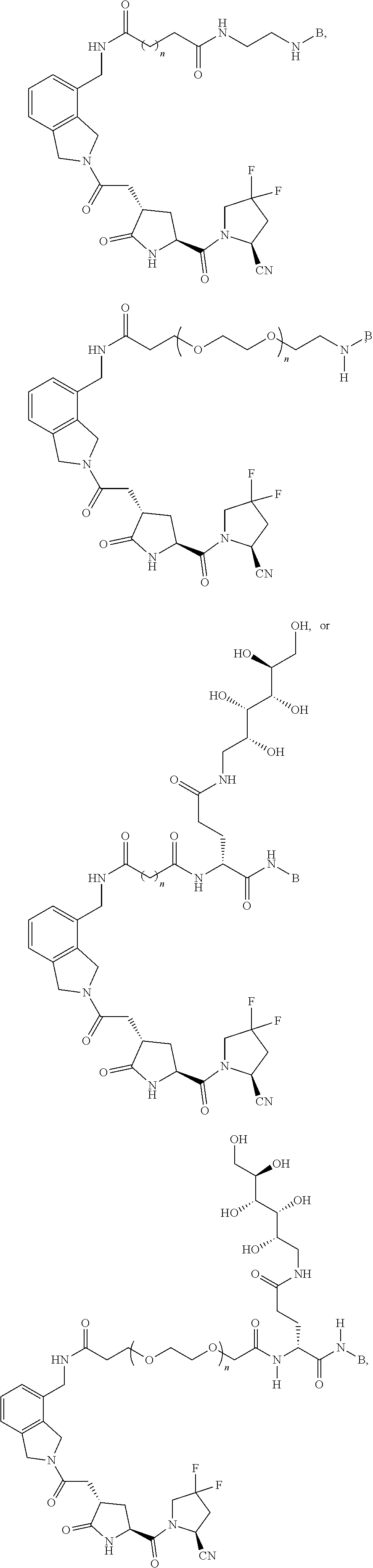

wherein:
n is an inter from 1-5; and
B is:

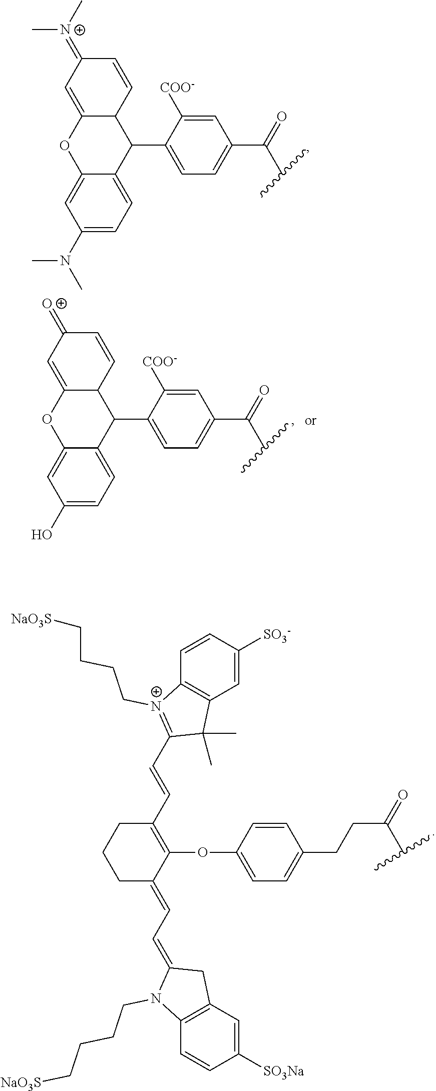

A-L-B can have the following structure:

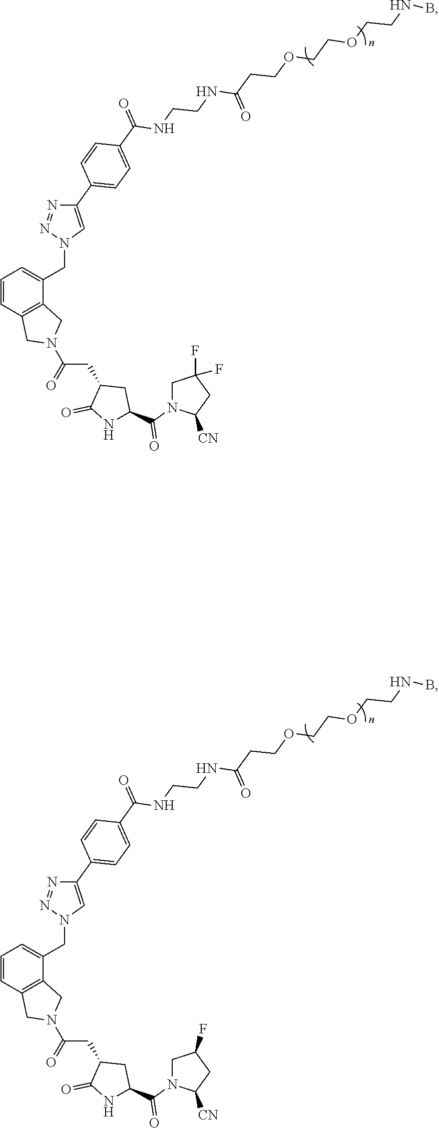

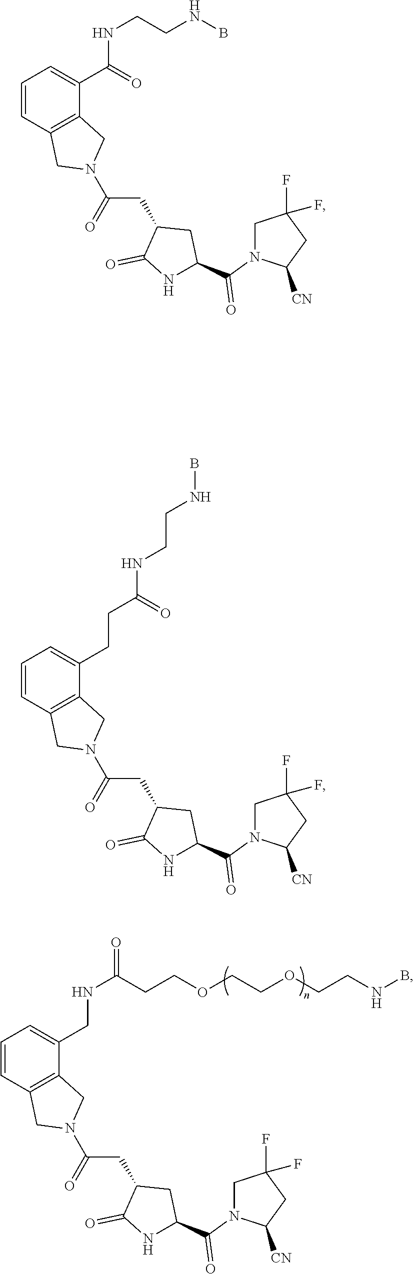

wherein:
n is an inter from 1-5; and
B is:

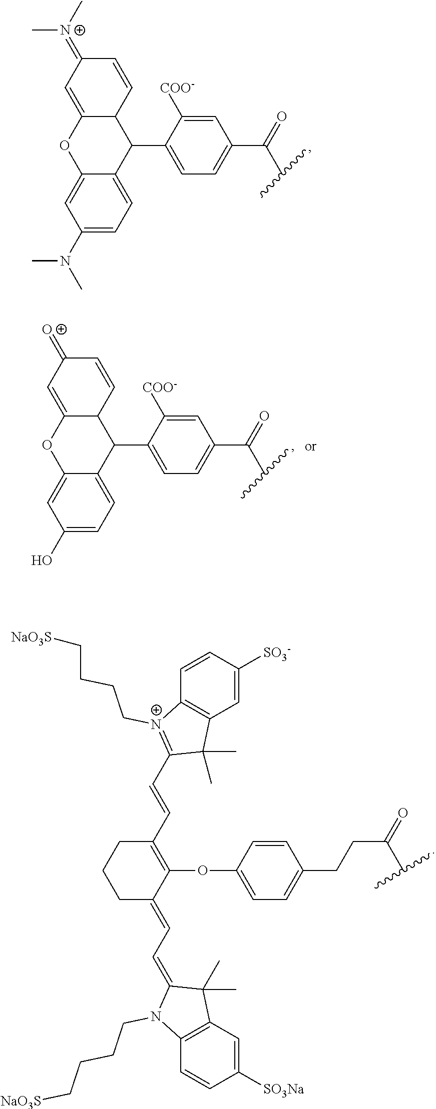

A compound (e.g., conjugate) can have the following structure:

TABLE 1

| Com- | |
|---|---|
| pound | Structure |
| 1 | 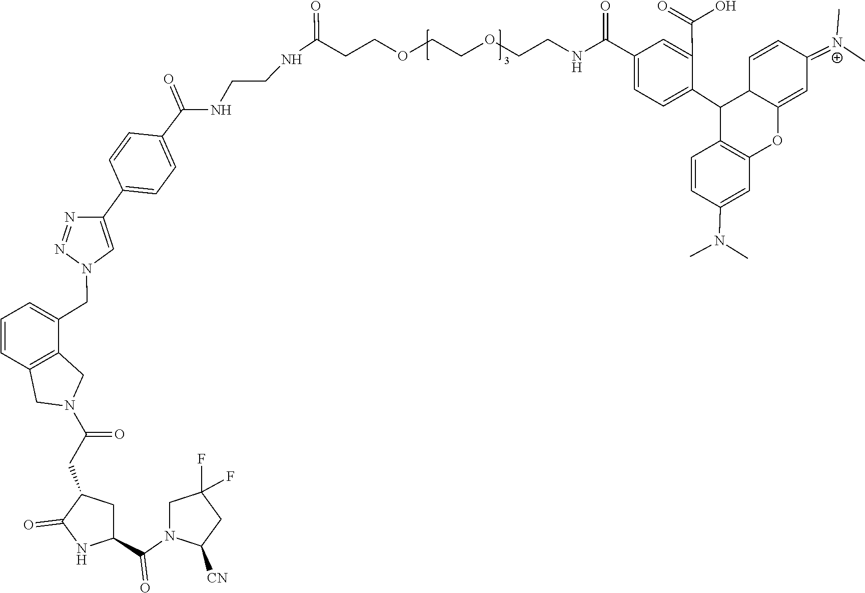 |
| 2 | 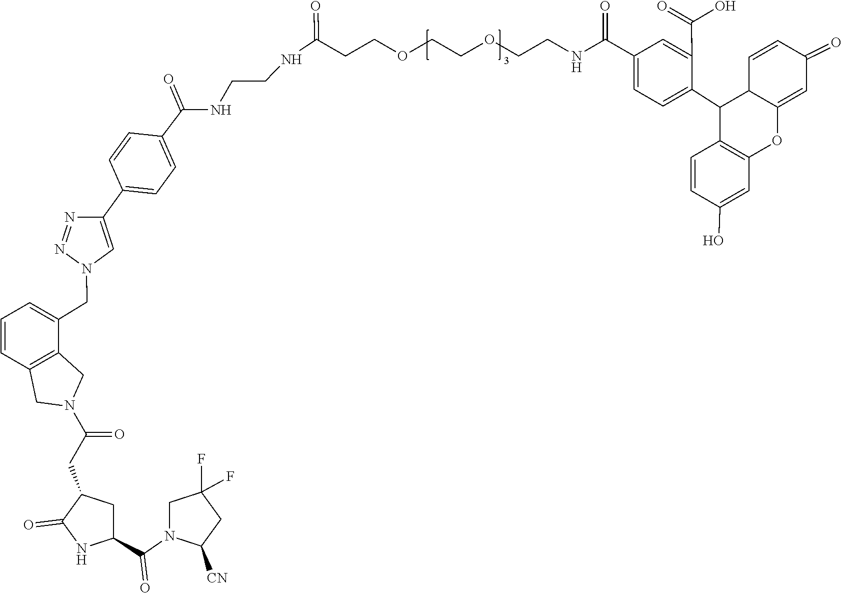 |
| 3 | 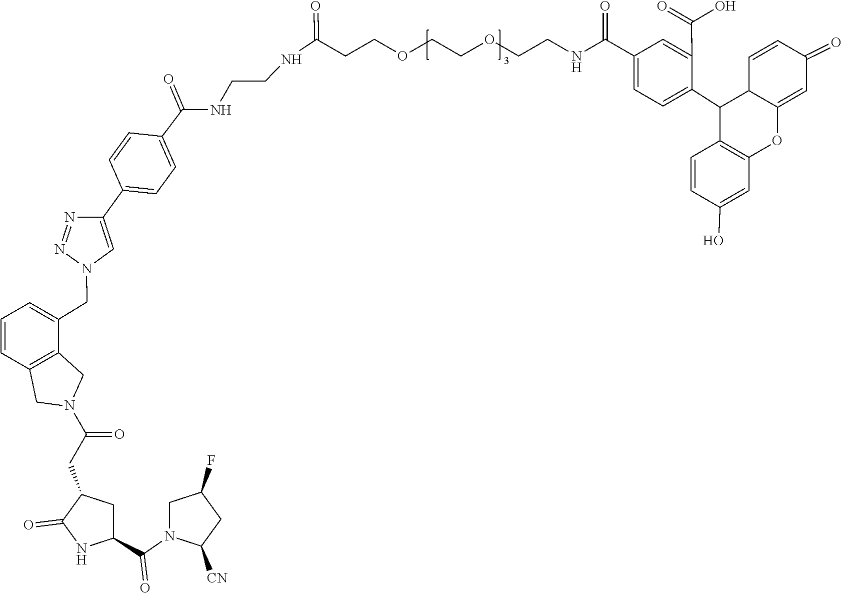 |
| 4 | 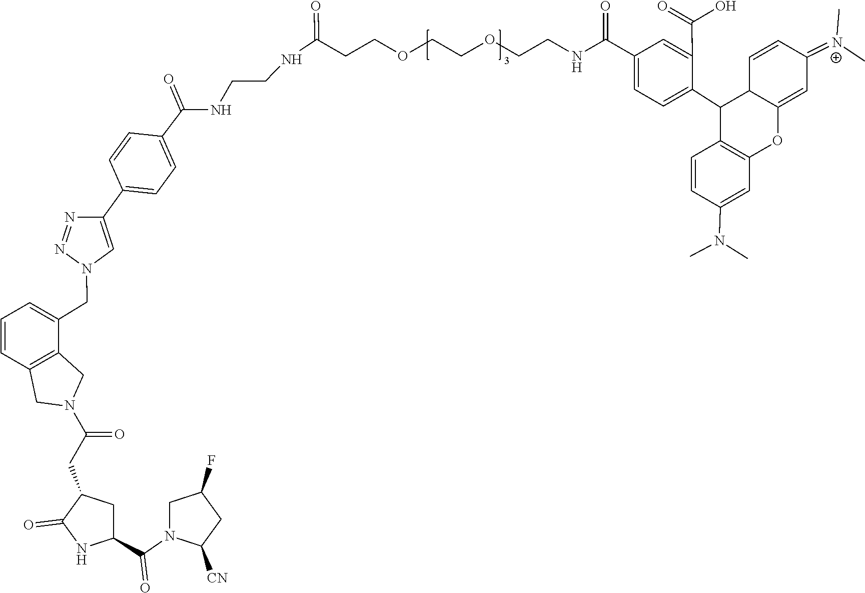 |
| 5 | 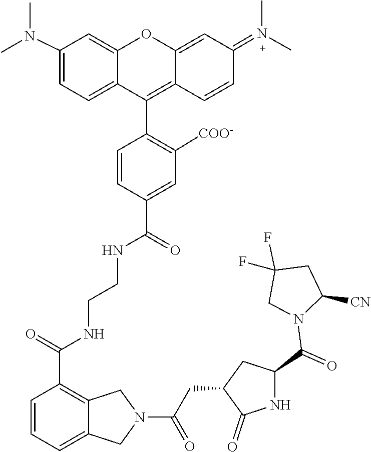 |
| 6 | 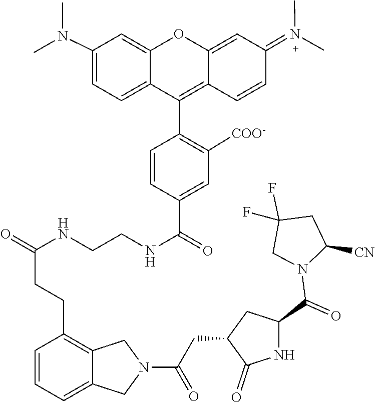 |
| 7 | 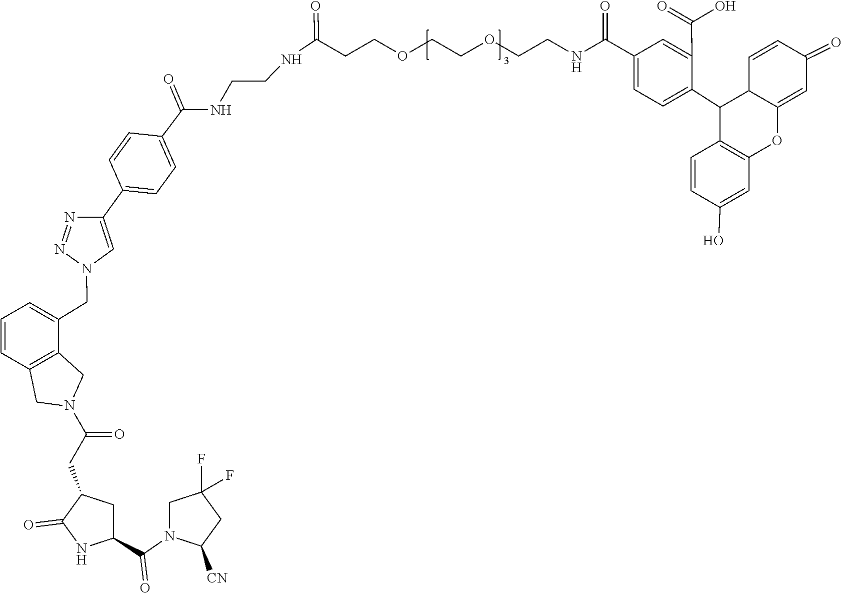 |
| 8 | 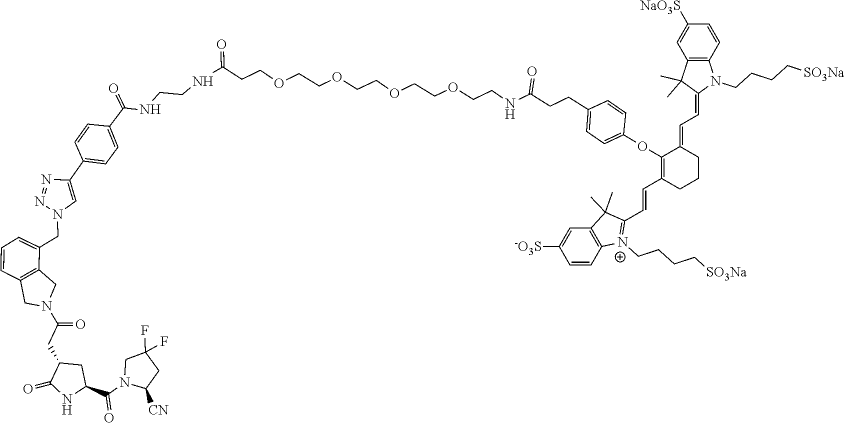 |
| 9 | 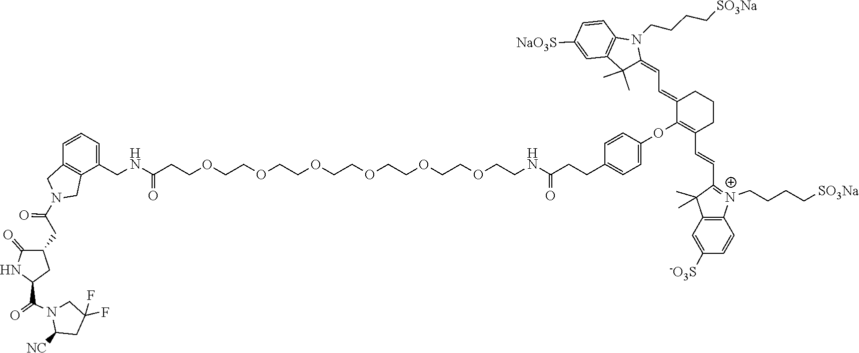 |
| 10 | 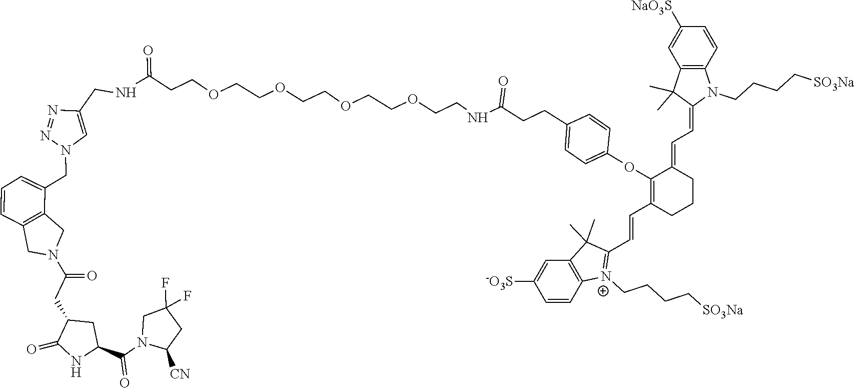 |

Below is a scheme illustrating a method for producing a non-labeled (competition) FAP ligand, such as used in the examples and figures herein:

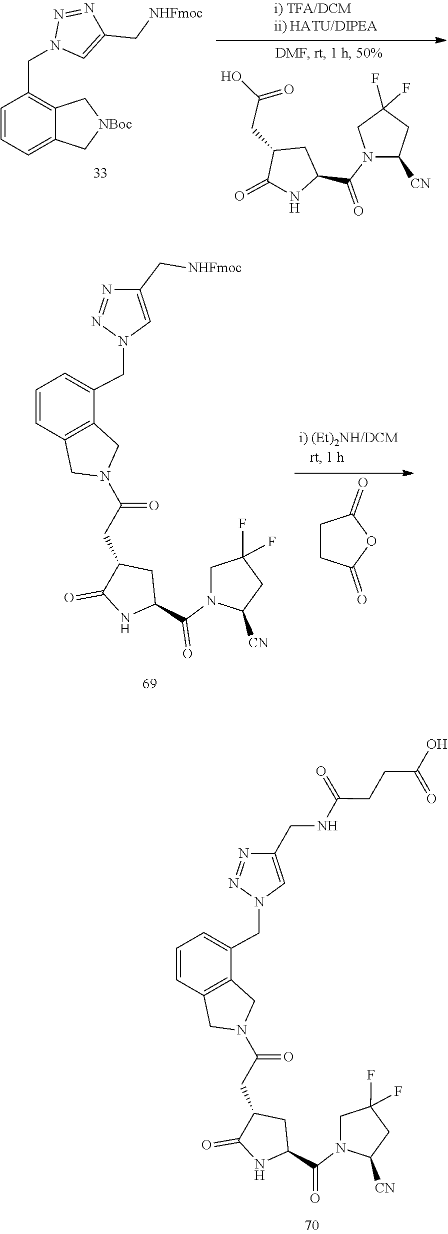

A shortcoming of certain therapeutic agents (e.g., chemotherapeutic agents or radiotherapeutic agents) is the inability of such agents to achieve and/or maintain therapeutically effective concentrations of the agent at a target location (e.g., at a cancer, a tumor, or a fibrotic tissue) without also providing unwanted, toxic and/or lethal (e.g., systemic) effects. General systemic or even local administration of such agents (e.g., that may be pathway-specific, but not target-tissue specific), results, in some instances, in off-target liabilities of the therapeutic agents, thereby increasing side effects. In some instances, a compound (e.g., such as a compound comprising a targeting ligand) that localizes a payload (e.g., a therapeutic agent) to a target site (e.g., a tumor or a fibrotic tissue) can improve residence time of the payload at the target site. In some instances, increasing residence time of a therapeutic payload at a target location facilitates increased and/or efficacious concentrations of the therapeutic agents, even at low or tolerable doses. In some instances, efficacious concentrations of the active payload are maintained at a target location for a time sufficient to achieve a therapeutic concentration (e.g., at tolerable doses and/or at doses that would not be sufficient to achieve a therapeutic concentration if the free payload were similarly administered) and/or for a time sufficient to reduce the frequency of dosing (e.g., relative to what would be needed for administration of the free payload). Moreover, increasing residence time of the therapeutic payload at the target location means, in some instances, that off-target effects can be reduced (e.g., because the active agent is maintained at the target location). In some instances, compounds facilitate administration of active agents or payloads with, for example, decreased dosing frequency, decreased side effects, or combinations thereof (e.g., relative to administration of an otherwise similar free active agent or payload).

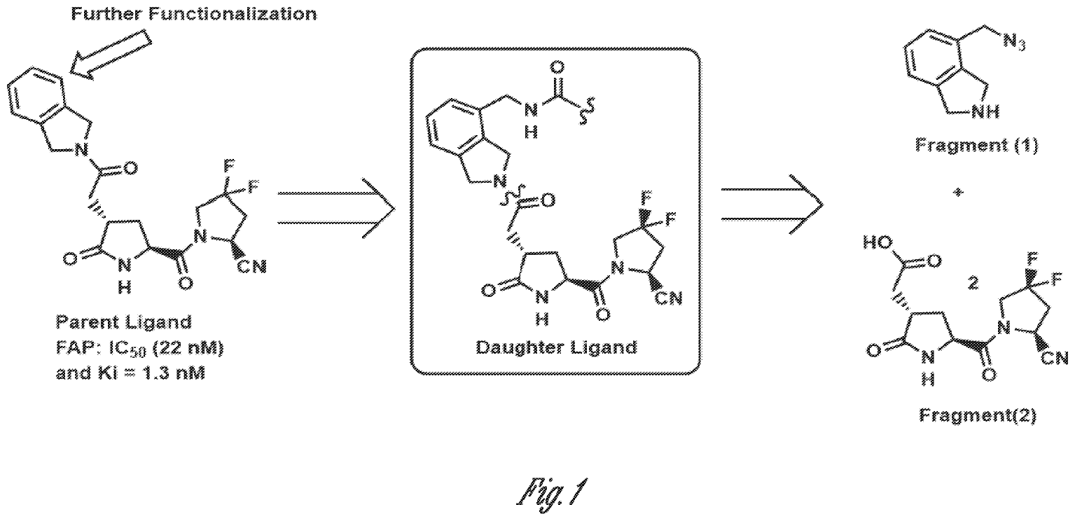

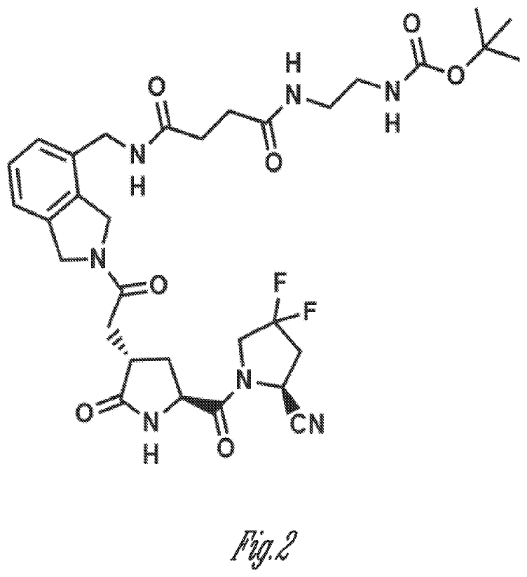

A targeting ligand provided herein is synthesized according to the retro-synthetic scheme shown in FIG. 1. The retro-synthetic scheme of FIG. 1 is used to synthesize a compound shown in FIG. 2. However, any suitable synthetic scheme or processes may be used to produce a compound. For example, certain synthetic schemes are illustrated in the Examples. All such synthetic schemes are incorporated into the detailed description herein for any process, step, or compound (e.g., end product of a scheme, intermediate of a scheme, and/or reagent of a scheme).

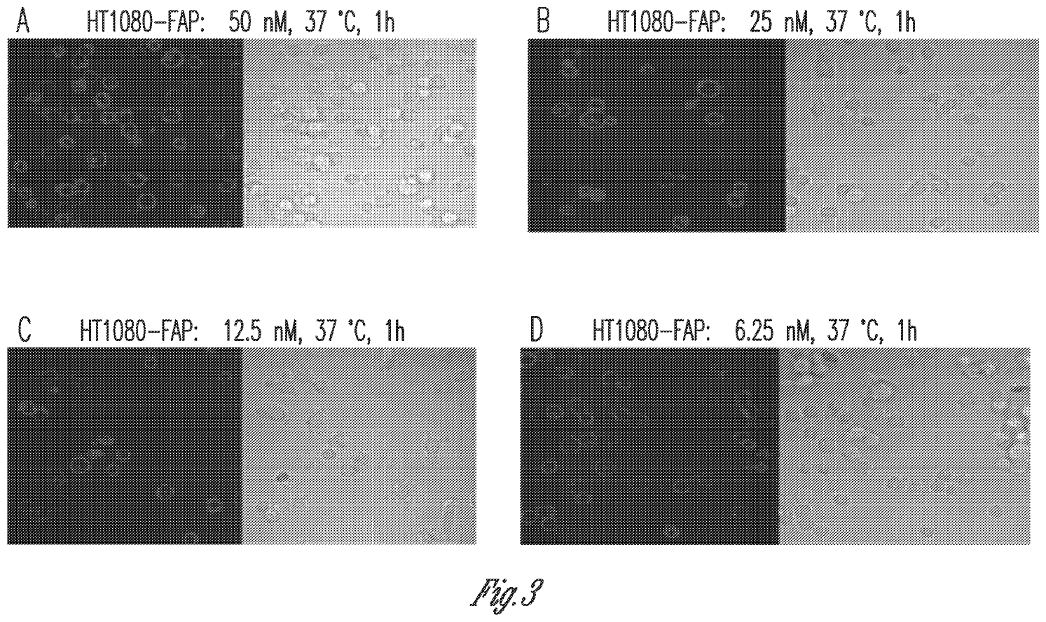

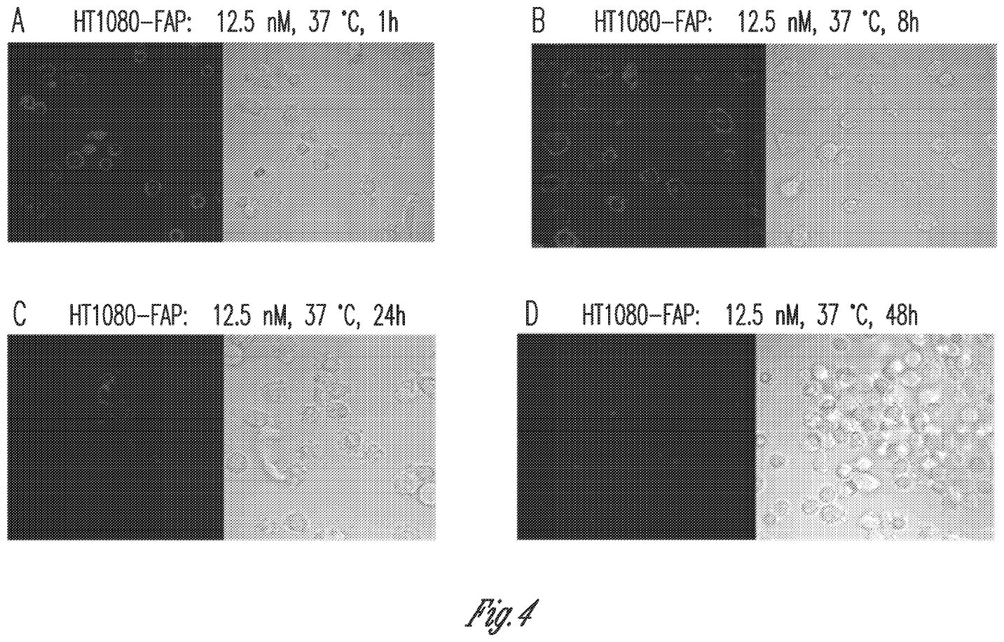

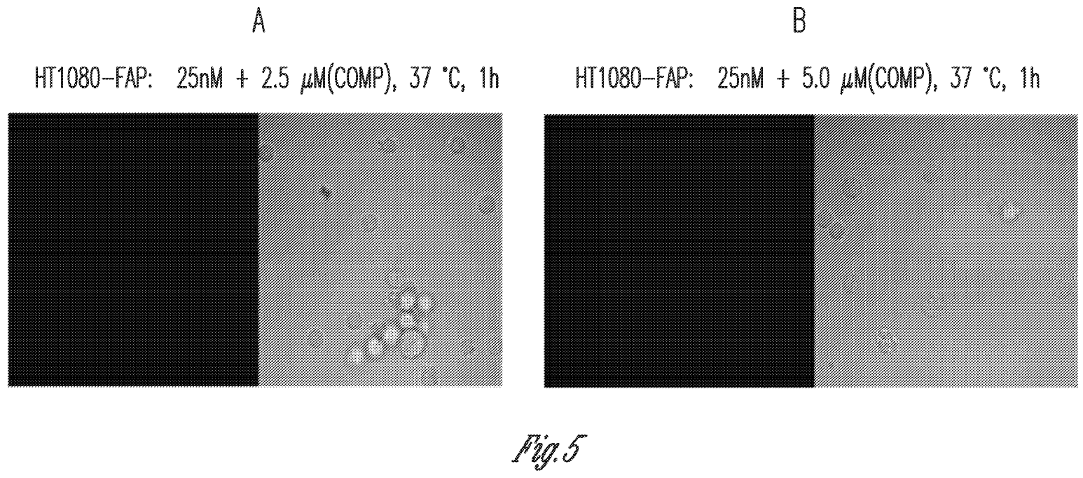

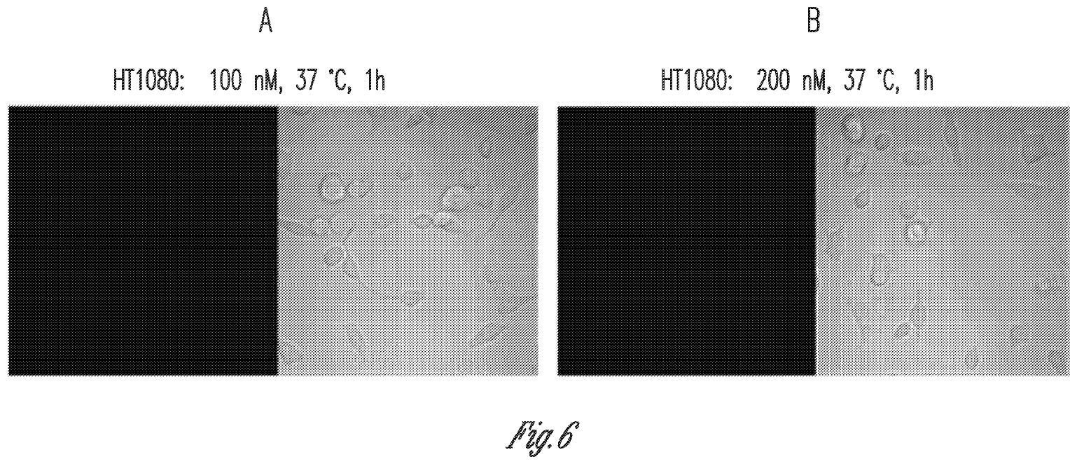

A compound targets (e.g., localizes to) a cell that expresses a FAP (e.g., FAP5). A compound binds to a FAP (e.g., FAP5) expressed by (e.g., and embedded within the cell membrane of) a cell (e.g., a cancer cell or a pro-fibrotic cell). For example, as shown by the grey shading in FIG. 3, FIG. 4, FIG. 5, and FIG. 6, compound 1 (at various concentrations, e.g., at 50 nM, 25 nM, 12.5 nM, and 6.5 nM (FIG. 3)) localizes to cells (e.g., HT1080-FAP cells) expressing FAP5. In addition, compound 1 localizes and internalizes the ligand to the membrane (e.g., where FAP5 is located) of the FAP5-expressing cells, remaining localized and internalized (e.g., when administered at a concentration of 12.5 nM) to the cells for at least 1 hour (e.g., 1 hour, 8 hours, 24 hours, or more) (e.g., FIG. 4).

FIG. 3 shows binding (e.g., at 50 nM (A), at 25 nM (B), at 12.5 nM (C), and at 6.25 nM (D)) of a targeting ligand on (FAP HT1080) cells for 1 hour. More surface binding is demonstrated with increasing concentrations of targeting ligand. FIG. 4 shows binding (e.g., at 12.5 nM) of a targeting ligand on FAP HT1080 cells for 1 hour (A), 8 hours (B), 24 hours (C), and 48 hours (D). At early time points, the compound (targeting ligand) is observed on the surface of the cells, with the compound being internalized into the cells over time. FIG. 5 shows binding of a targeting ligand on FAP HT1080 cells with at least 100-fold excess of competition ligand (e.g., A: 25 nM targeting ligand, 2.5 μM competitor; B: 25 nM targeting ligand, 5 μM competitor) for 1 hour. FIG. 6 shows binding (e.g., at 100 nM (A) and at 200 nM (B)) of a targeting ligand on non-FAP HT1080 cells. At comparable time points, little compound (targeting ligand) is observed on the surface of such cells after 1 hour (compared to FIGS. 3-5, which show good surface binding of the compound after a similar time on cells with high FAP surface concentrations, even at much lower concentrations).

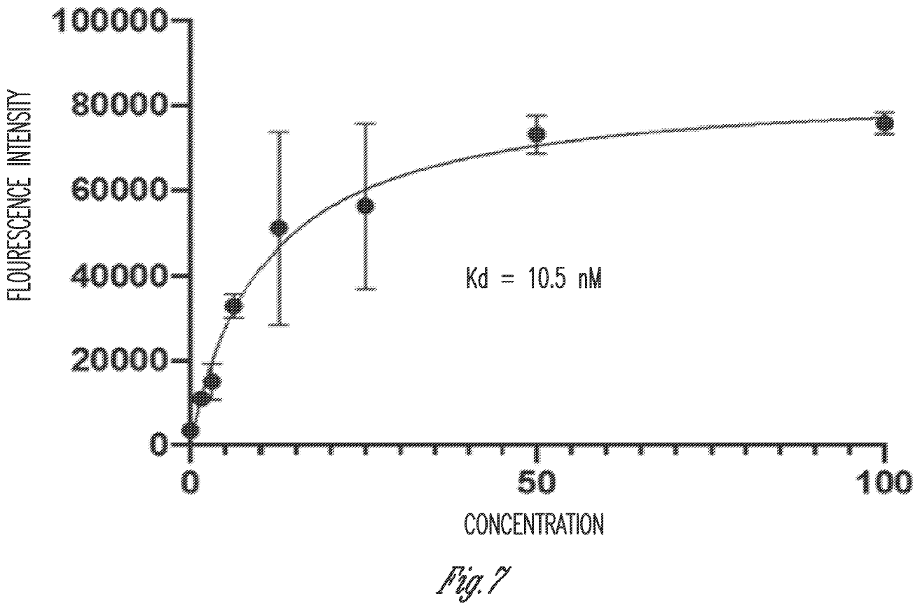

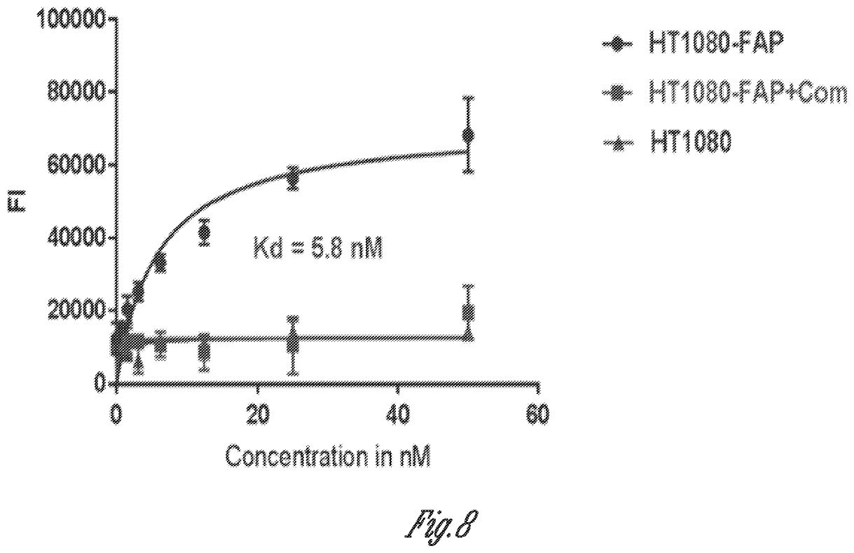

A compound can have a strong (e.g., binding) affinity to FAP (e.g., FAP5) expressing cells. In some instances, this strong binding affinity allows the compounds to localize to the FAP (e.g., FAP5) expressing cells for a prolonged period of time (e.g., for a period of time sufficient to measure a signal from the compound localized to a tissue of interest (e.g., a tumor or fibrotic tissue) or deliver the payload (e.g., the therapeutic agent)). A compound can have a binding affinity to FAP (e.g., FAP5) expressing cells from 0.01 nM to 1 μM. For example, compound 1 has a K_{d} from 5 nM to 15 nM in cells expressing FAP5 (FIG. 7). Furthermore, as shown in FIG. 8, compound 1 does not bind to HT1080 cells that do not express FAP5 and compound 1 does not bind to HT1080 cells expressing FAP5 in the presence of an unlabelled FAP5 ligand (e.g., compound 8).

FIG. 7 shows a binding curve for a targeting ligand on HT1080-FAP cells. FIG. 8 shows binding curves for a targeting ligand on HT1080-FAP cells (targeting ligand only (circles) and targeting ligand and competitor (squares)) and HT1080 cells (triangles).

A compound can target a cancer cell (e.g., or a tumor). A compound provided herein can target a tumor with minimal off-target effects. A compound can accumulate rapidly and can maintain its concentration in the tumor for long periods of time. As illustrated, ligands demonstrate good accumulation in the target location (tumor), while demonstrating limited accumulation at off-target locations. As further illustrated, there is little or no accumulation of the compound (FAP ligand conjugate) in non-tumor organs. Specifically, as illustrated, in some instances, high concentrations of compounds can be achieved at the target location (e.g., tumor) for extended periods of time (e.g., up to 5 days or more), while having little or no accumulation at any point in the heart, liver, lungs, spleen, stomach, or intestines. There is some accumulation in the kidneys for a short period of time, but that is mostly cleared by 15 hours (e.g., compared to the having high concentrations at the target location for days). In certain instances, this means that certain therapies, which, without the targeting ligands, would need to be administered once or twice daily to maintain a therapeutic concentration and/or provide a therapeutic effect (if such results are even obtainable without non-tolerable side effects and/or death), can be administered in a form provided herein at a much less frequent rate (e.g., once or twice a week). Moreover, as noted, there is little off-target accumulation of compounds provided herein, indicating tolerability of dosing of such compounds in efficacious amounts. In some instances, a compound accumulates in the kidneys of a subject. In some instances, the accumulation of the compound (e.g., in the kidneys) is at most 6 hours. In some instances, the compound is significantly cleared from the organ (e.g., the kidney) by 24 hours (e.g., by 24 hours, by 15 hours, or the like). In some instances, the compound remains localized to the tumor for at least 1 day (e.g., 1 day or more, 2 days or more, 3 days or more, 4 days or more, 5 days or more, or the like).

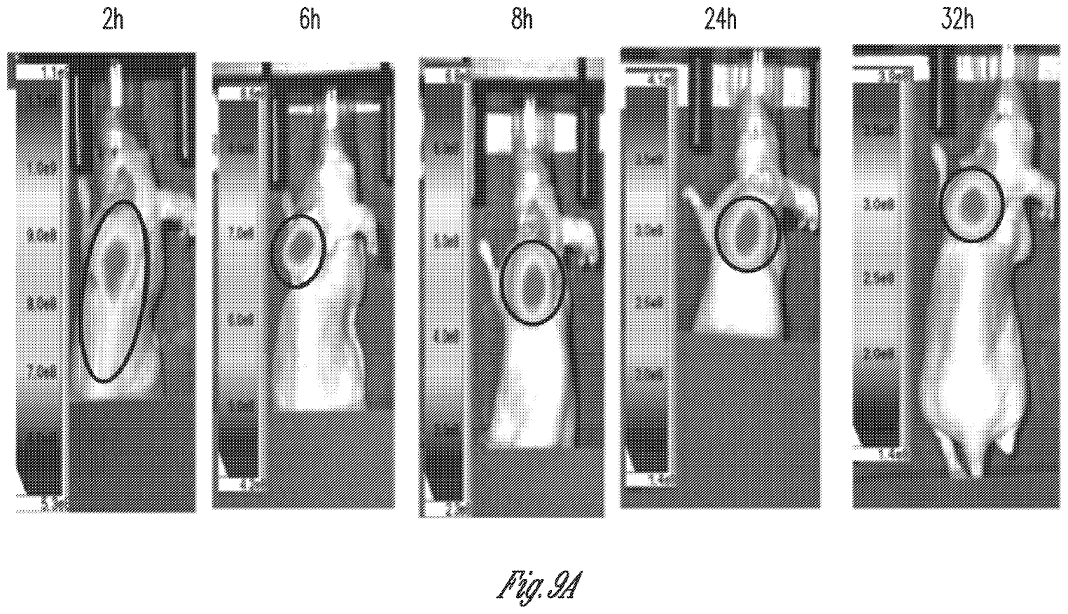

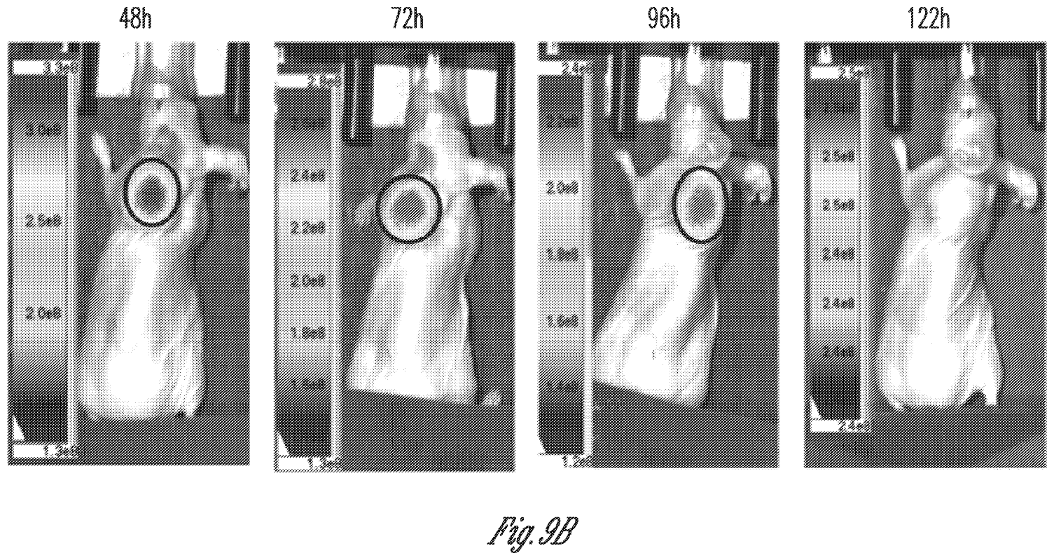

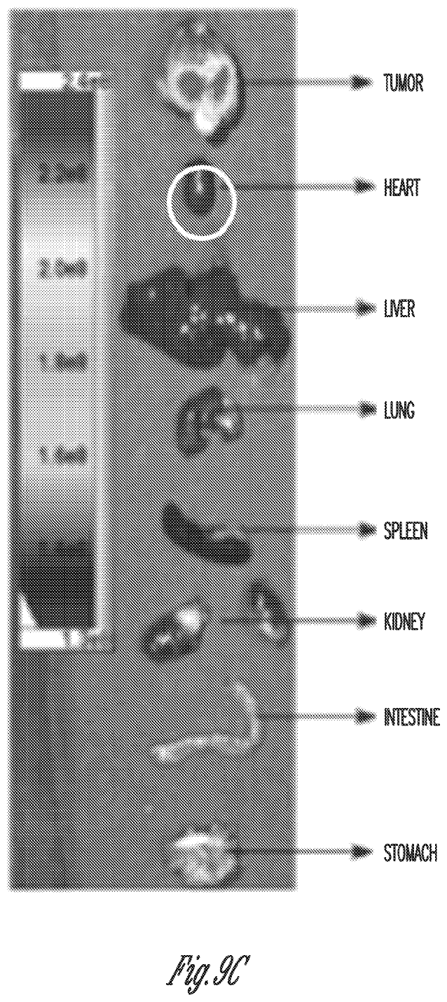

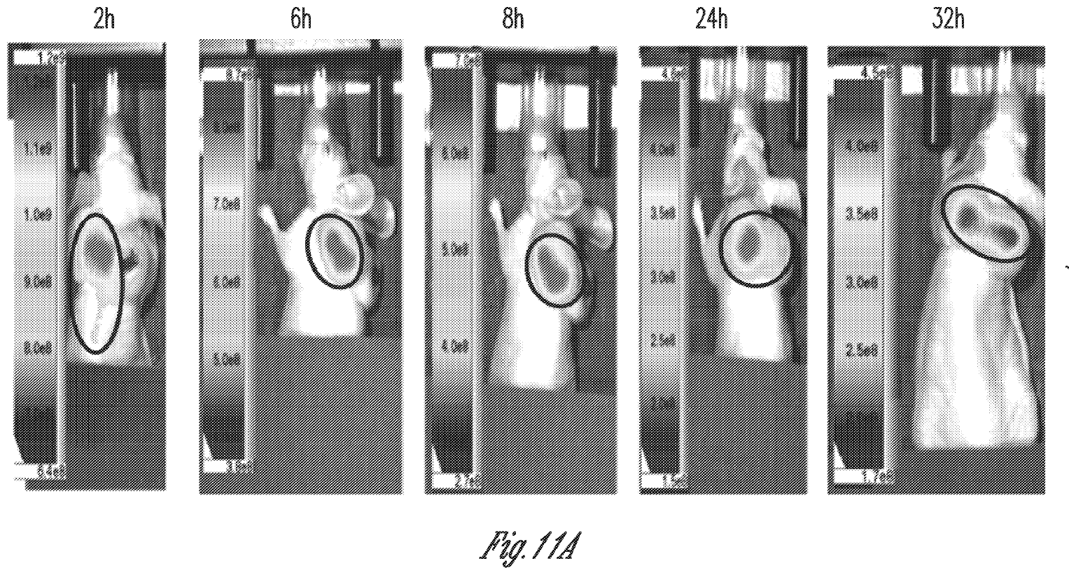

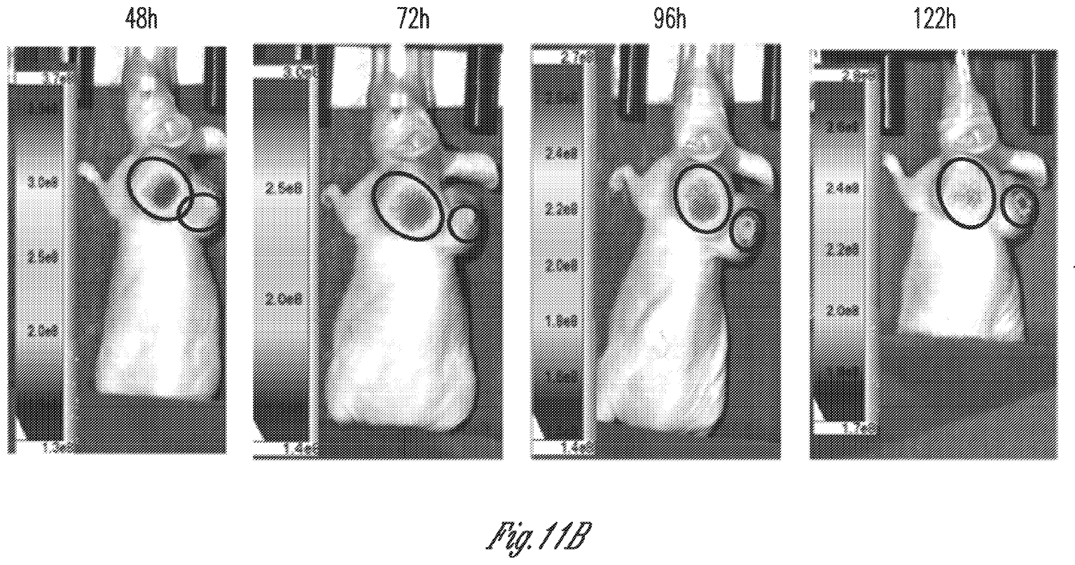

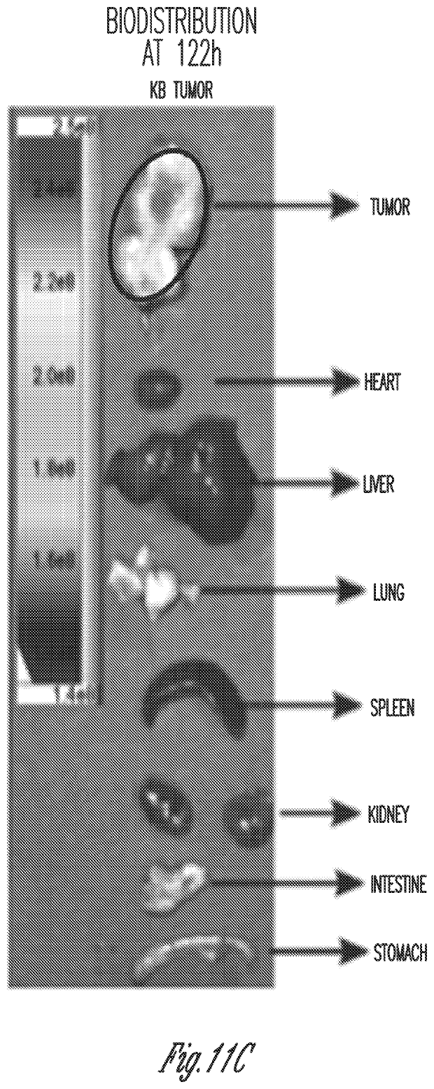

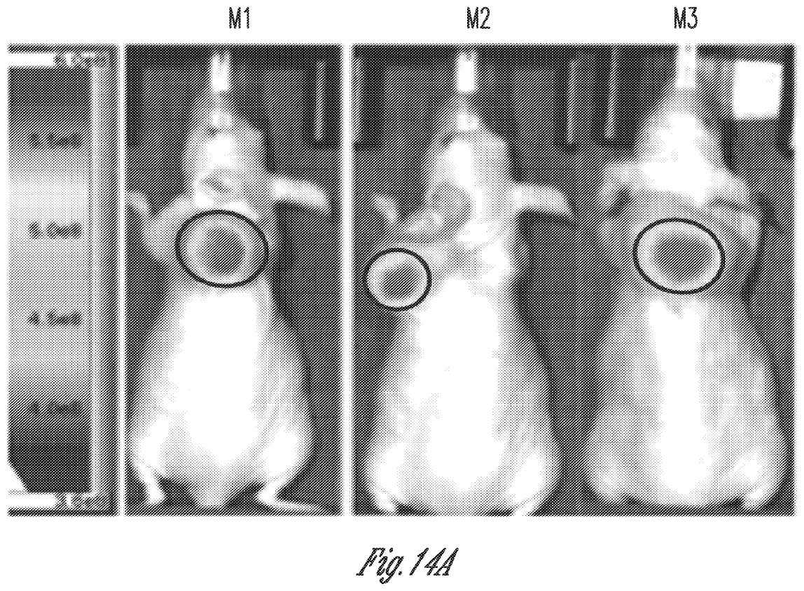

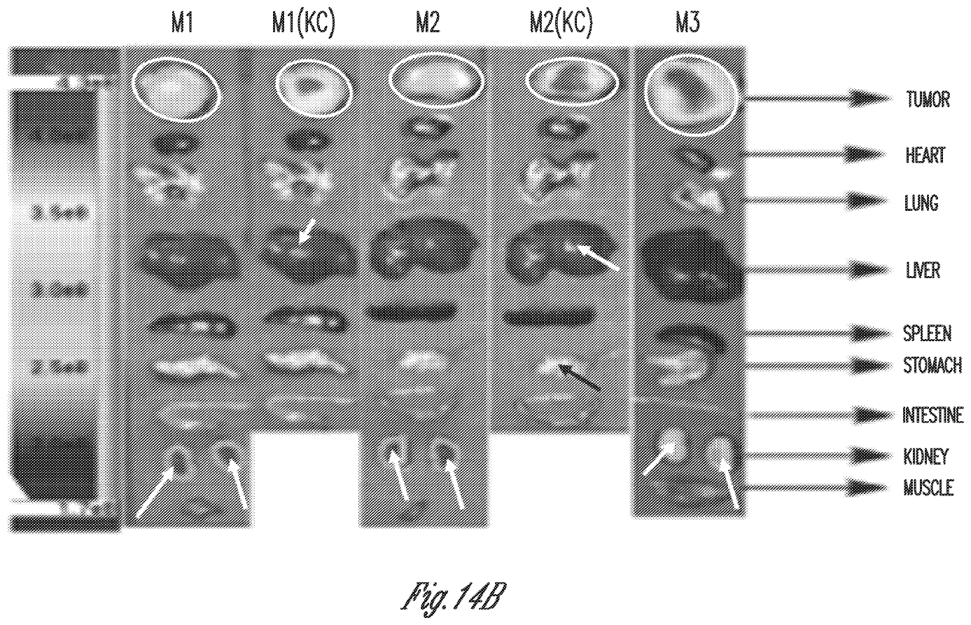

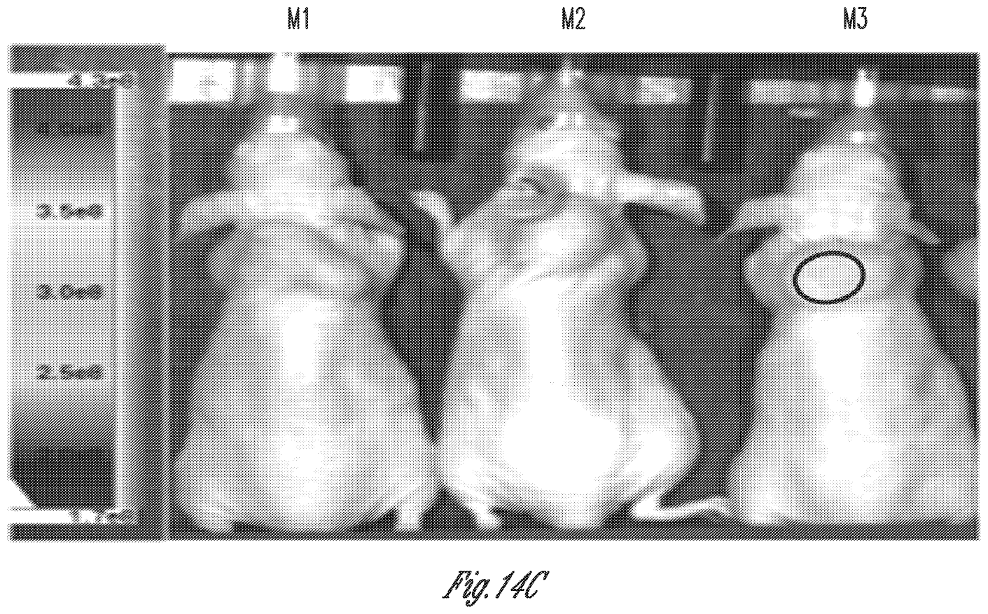

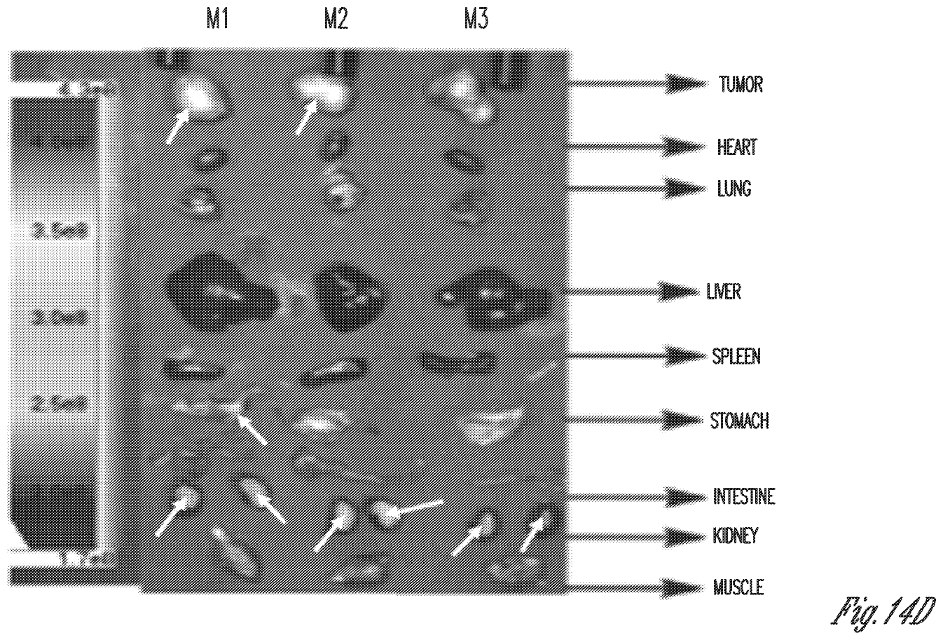

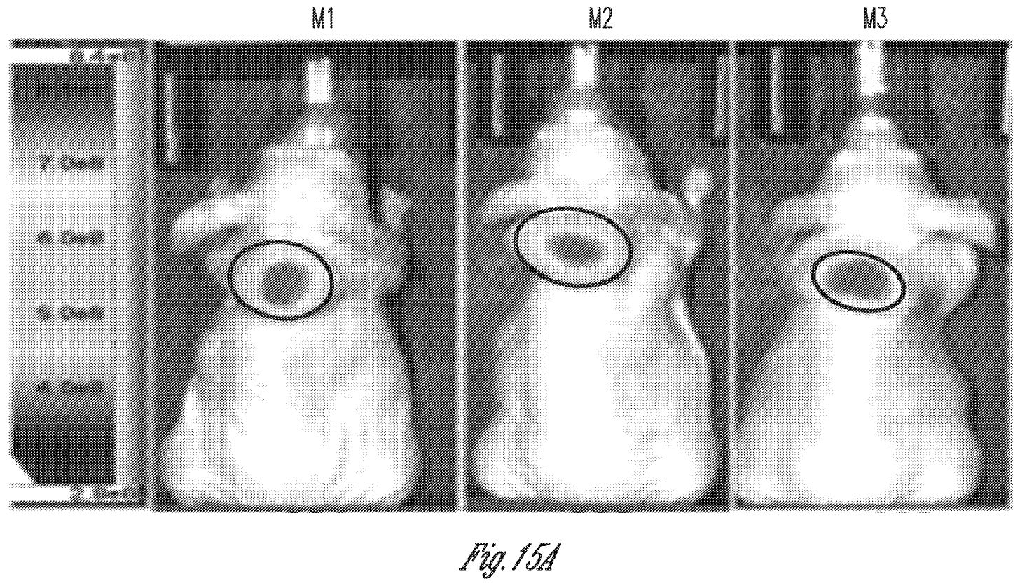

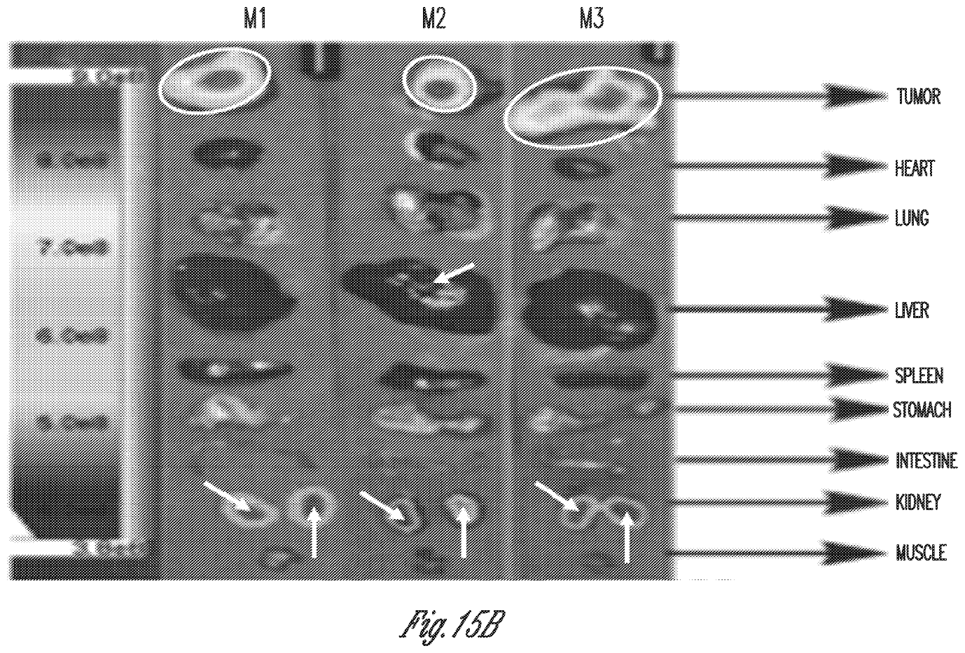

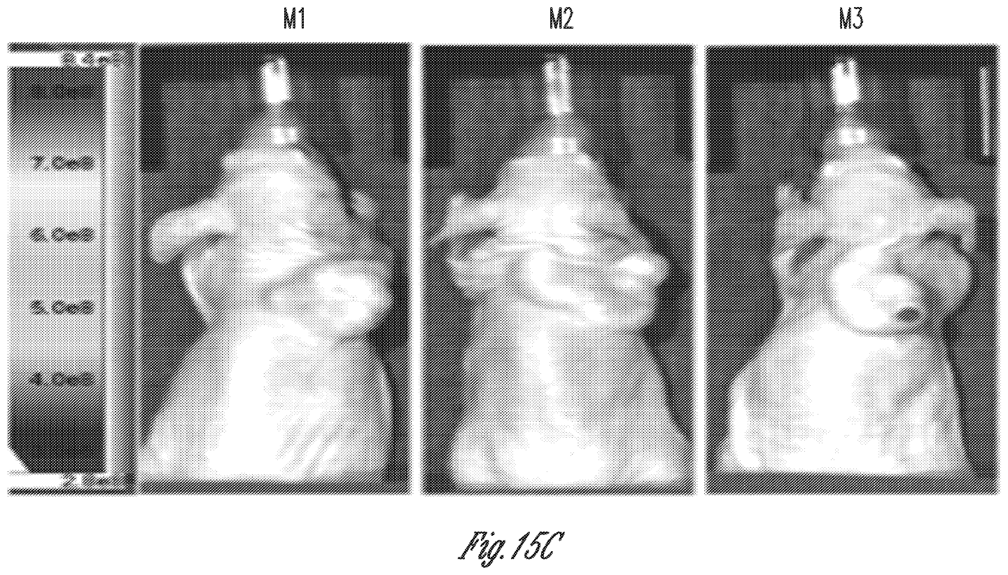

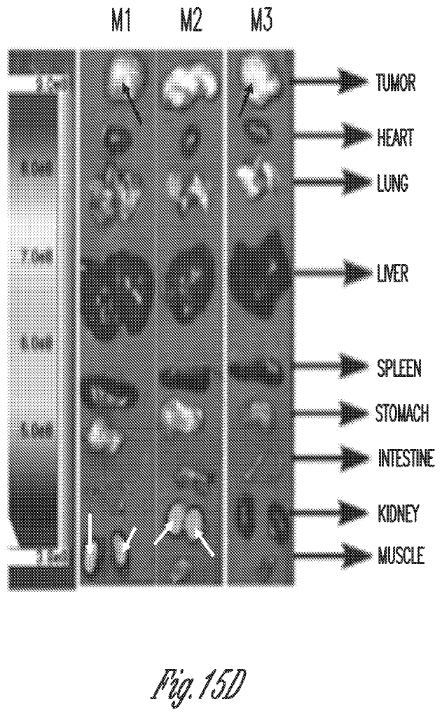

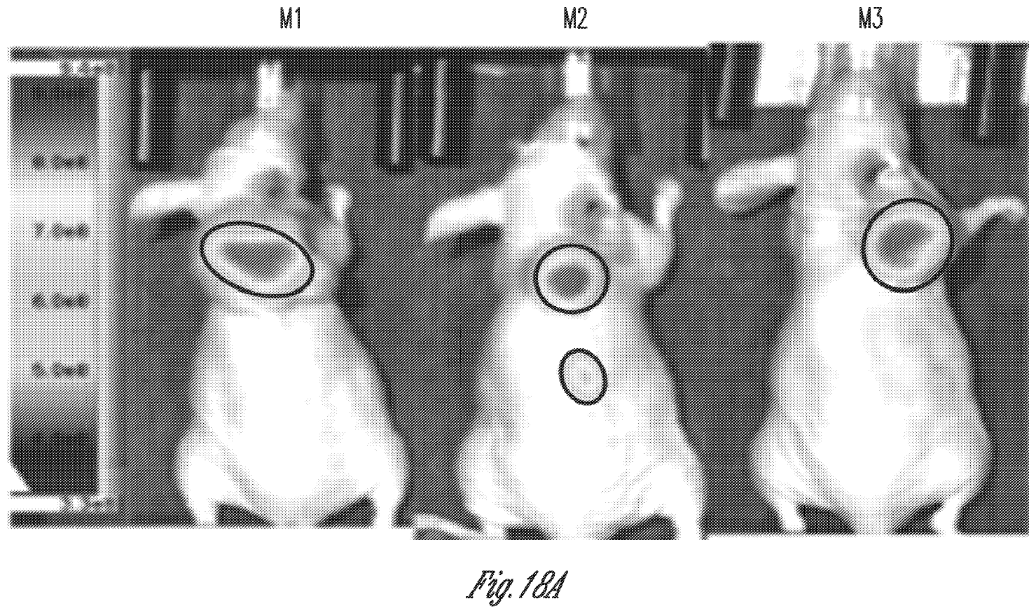

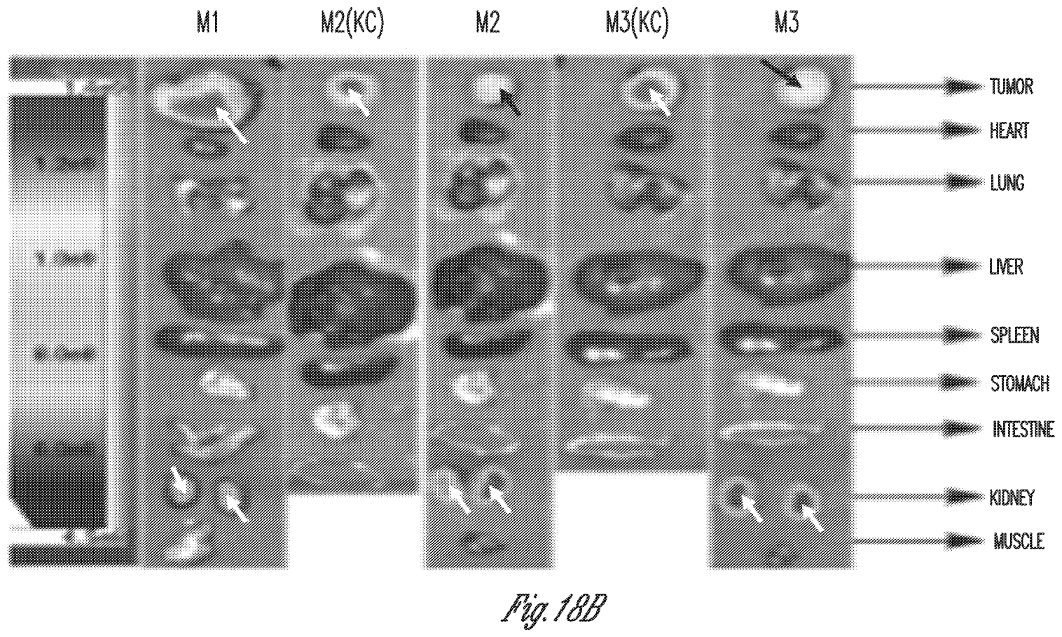

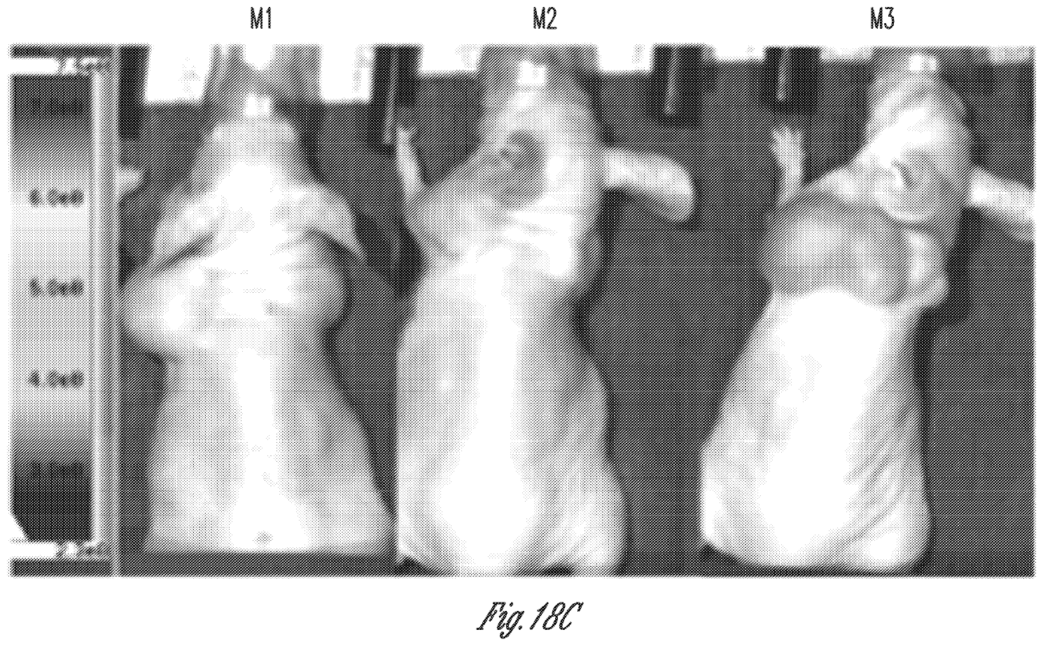

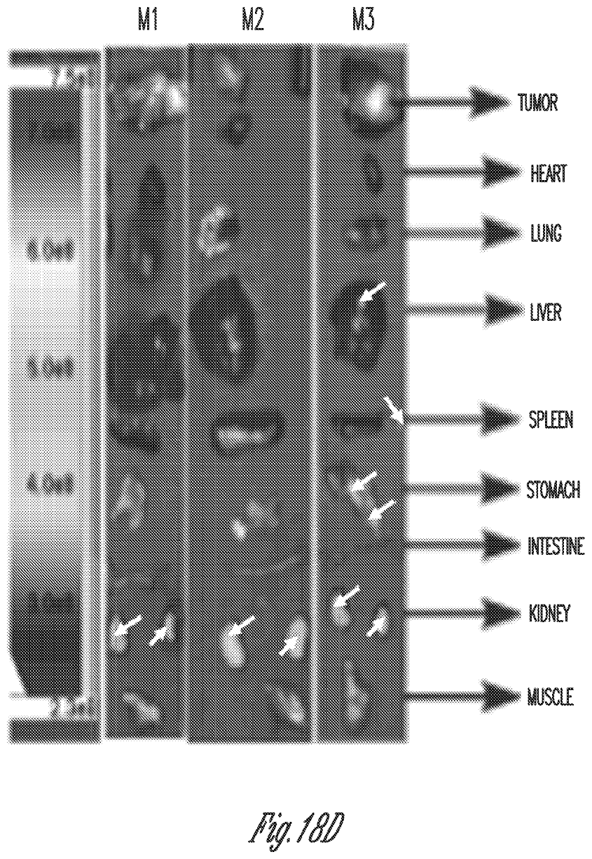

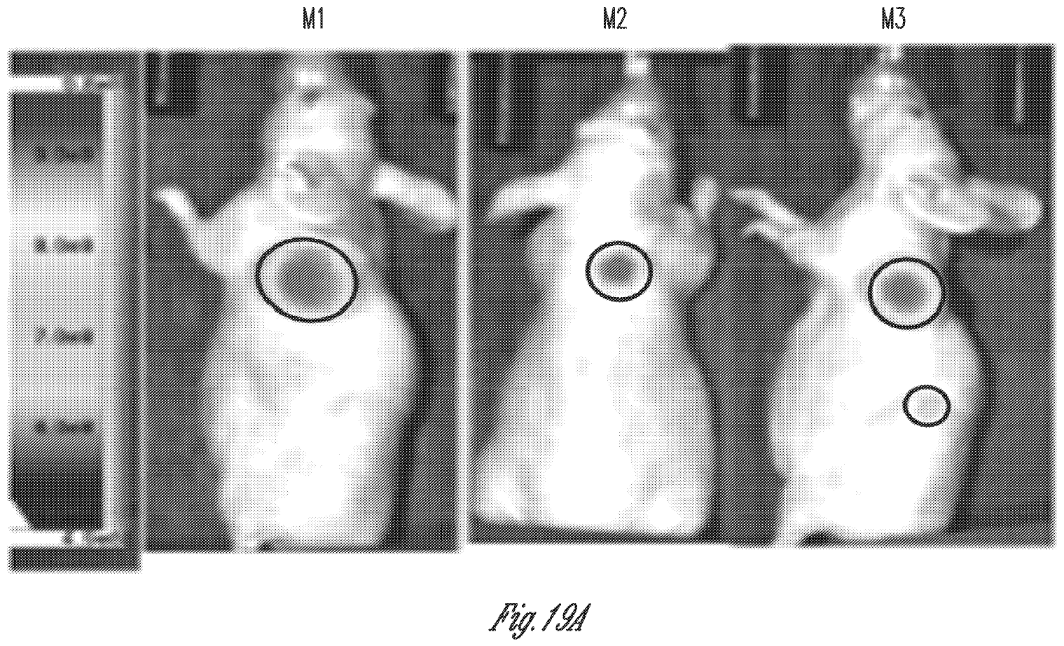

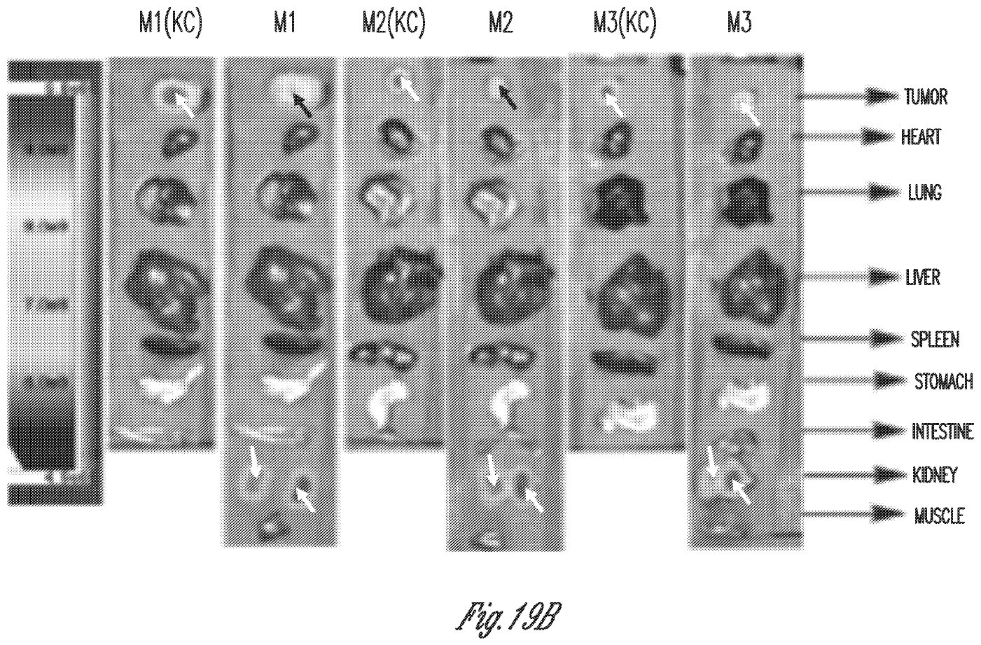

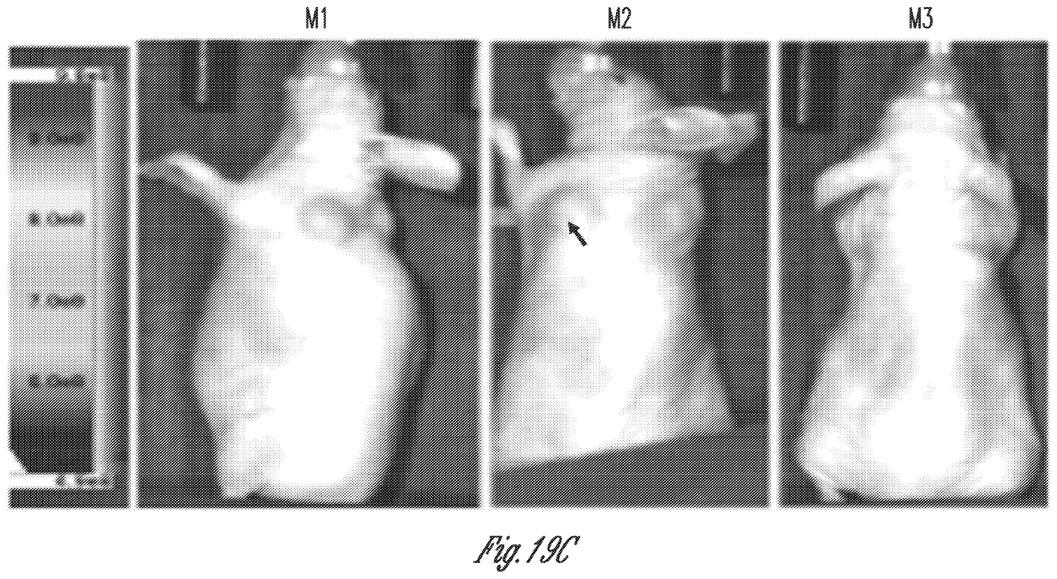

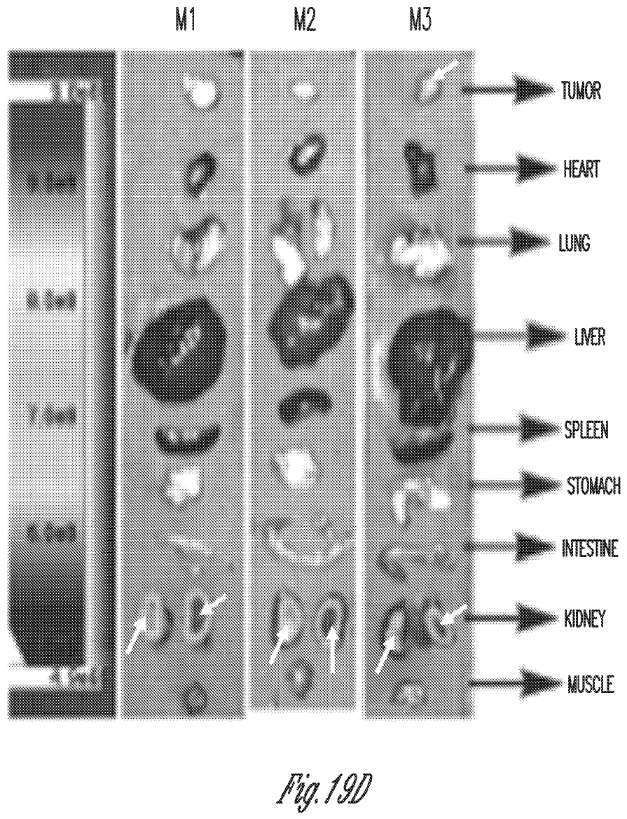

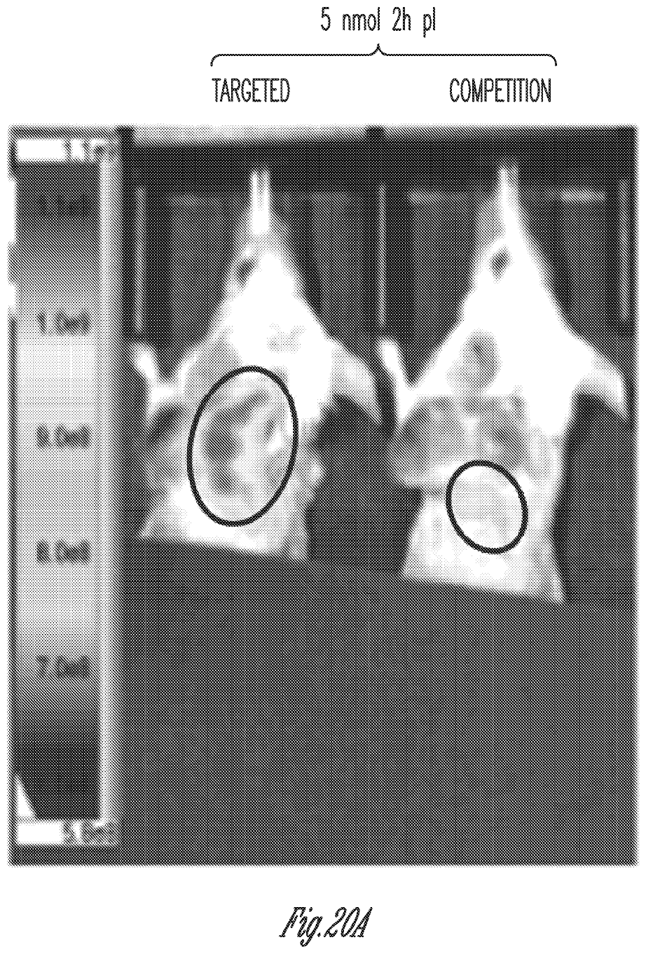

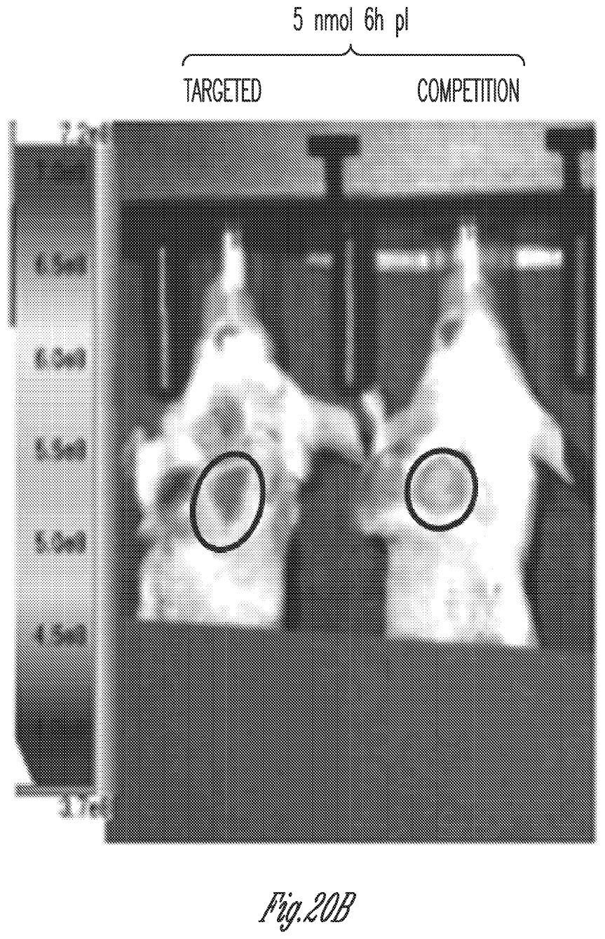

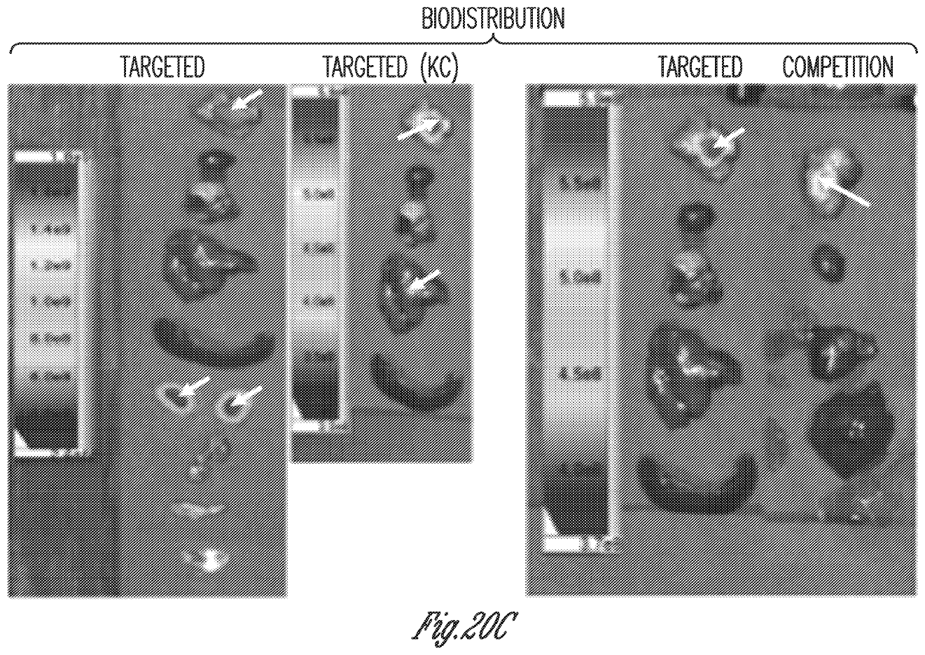

For example, FIG. 9A-FIG. 20C show that a compound (e.g., compound 8) localizes to a tumor (e.g., expressing FAP5 in MDA-MB-231 xenograft mice (e.g., FIGS. 9A-10 and FIGS. 17A-17D), KB xenograft mice (e.g., FIGS. 11A-13D and FIGS. 16A-16D), FADu xenograft mice (e.g., FIGS. 14A-14D), HT29 xenograft mice (e.g., FIGS. 15A-15D), U87MG tumor xenograft mice (e.g., FIGS. 18A-18D), PANC1 xenograft mice (e.g., FIGS. 19A-19D), and 4T1 tumor xenograft mice (e.g., FIGS. 20A-20C)). This data shows that a compound provided herein localizes to the tumor (only) (e.g., in several tumor types) for at least 1 day, 2 days, 3 days, 4 days, 5 days, or more. Furthermore, while the compound (e.g., compound 8) does show some build-up in the kidneys (e.g., generally starting at 2 hours), the peak build-up in the kidneys is at 6 hours, with rapid reduction and clearance thereafter. For example, significant reduction of the kidney exposure is achieved after just 15 hours, with almost complete clearance by 24 hr. By contrast, the compound is quickly taken up (e.g., by 2 hours) and maintained in the tumor for much longer than (e.g., for more than 1 day, 2 days, 3 days, 4 days, 5 days, or the like) its presence in the kidney. Together, these in vivo data show that the conjugates provided herein target the kidneys, allowing for the delivery of the payload (e.g., the imaging agent or the chemotherapeutic agent) for a prolonged period (e.g., for several days).

FIG. 9A and FIG. 9B show the in vivo imaging of a targeting ligand provided herein (e.g., at a dose of 10 nmol) on MDA-MB-231 xenograft mice (having a tumor size of 400 mm³) for 2 hours to 122 hours. The biodistribution in the tumor, heart, liver, lung, spleen, kidney, intestine, and stomach after 122 hours is shown in FIG. 9C. Black or white ovals or circles in the images highlight where the targeting ligand is present. Darker shading within the oval or circle represents a higher concentration of targeting ligand than the lighter shading within the oval or circle. More generally, FIG. 9A and FIG. 9B illustrate imaging results demonstrating in vivo tumor-specific targeting of a targeting ligand after administration to a mammal having a tumor with a FAP-rich environment for 2 hours to 122 hours. FIG. 9C illustrates the biodistribution of a targeting ligand after administration to a mammal having a tumor with a FAP-rich environment in the tumor, heart, liver, lung, spleen, kidney, intestine, and stomach after 122 hours. As can be seen, there is good targeting of the tumor (i) for several days and (ii) with little to no compound found elsewhere when observing the mammal generally or the organs of the mammal specifically.

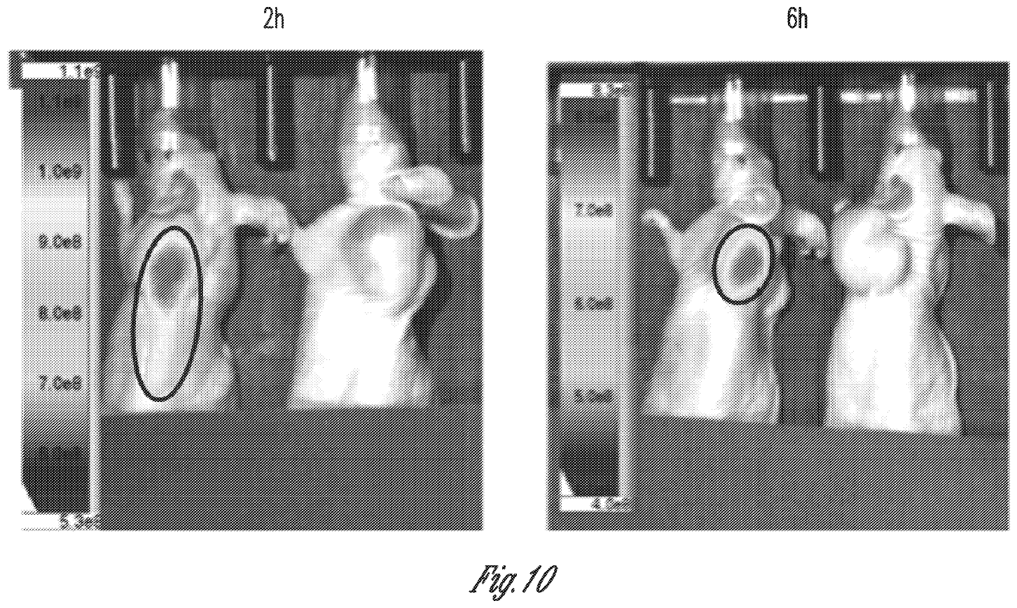

FIG. 10 shows the in vivo imaging of a targeting ligand provided herein on MDA-MB-231 xenograft mice for 2 hours to 6 hours both with and without an unlabelled competitor. In studies without competitor, 10 nmol of the labelled ligand were administered to mice. In studies with competitor, 10 nmol of the labelled ligand and 1,000 nmol of the unlabelled ligand were administered to the mice. For each timepoint, the left-most mouse represents the mouse treated with targeting ligand only while the right-most mouse represents the mouse treated with targeting ligand and unlabelled competitor. Black ovals or circles in the images highlight where the targeting ligand is present. Darker shading within the oval or circle represents a higher concentration of targeting ligand than the lighter shading within the oval or circle. As can be seen, without a FAP competitor, good targeting of the tumor location is achieved after 2 and 6 hours. By contrast, presence of FAP competitor limited the tumor targeting of the compound, demonstrating that the ability of the compound to target FAP is important, in some instances, for a compound to be able to target a desired location. Moreover, in some instances, such information demonstrates that locations that are not FAP-rich are not expected to achieve disproportionate or unwanted accumulation of a targeted compound.

FIG. 11A and FIG. 11B show the in vivo imaging of a targeting ligand provided herein (e.g., at a dose of 5 nmol) on KB xenograft mice (having a tumor size of 600 mm³) for 2 hours to 122 hours. The biodistribution in the tumor, heart, liver, lung, spleen, kidney, intestine, and stomach at 122 hours is shown in FIG. 11C. Black ovals or circles in the images highlight where the targeting ligand is present. Darker shading within the oval or circle represents a higher concentration of targeting ligand than the lighter shading within the oval or circle. High concentrations of active agent are seen at the tumor location for 5 days, or more. By contrast, other organs are demonstrably free of FAP-targeting compound. Moreover, these data demonstrate similar effects in different systems, including the MDA-MB-231 xenograft mouse of FIG. 9 and the KB xenograft mouse of FIG. 11.

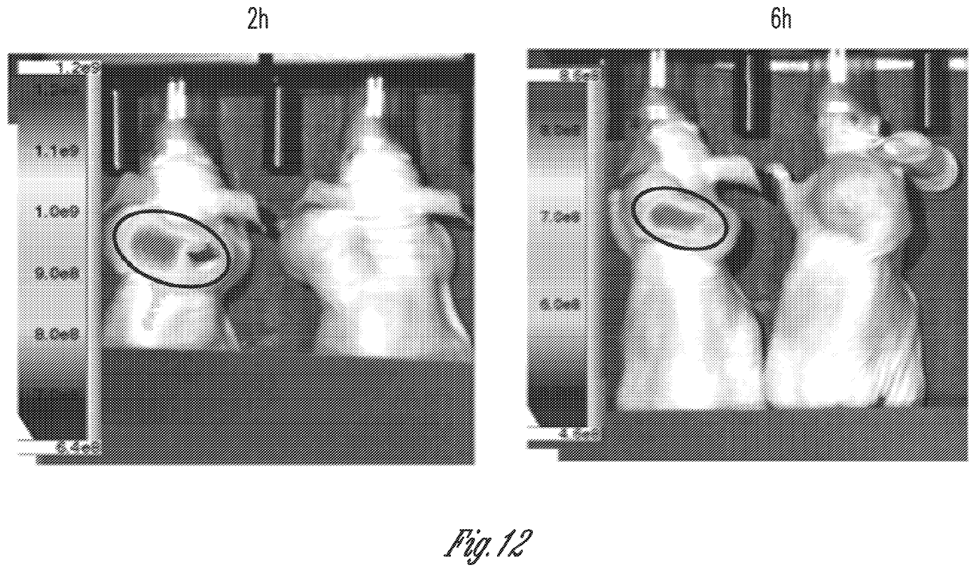

FIG. 12 shows the in vivo imaging of a targeting ligand provided herein on KB xenograft mice for 2 hours to 6 hours both with and without an unlabelled competitor. In studies without competitor, 10 nmol of the labelled ligand was administered to mice. In studies with competitor, 10 nmol of the labelled ligand and 1,000 nmol of the unlabelled ligand were administered to the mice. For each timepoint, the left-most mouse represents the mouse treated with targeting ligand only, while the right-most mouse represents the mouse treated with targeting ligand and unlabelled competitor. Black ovals or circles in the images highlight where the targeting ligand is present. Darker shading within the oval or circle represents a higher concentration of targeting ligand than the lighter shading within the oval or circle. As can be seen, without a FAP competitor, good targeting of the tumor location is achieved after 2 and 6 hours. By contrast, presence of FAP competitor limited the tumor targeting of the compound, demonstrating that the ability of the compound to target FAP is important, in some instances, for a compound provided herein to be able to target a desired location. Moreover, in some instances, such information demonstrates that locations that are not FAP-rich are not expected to achieve disproportionate or unwanted accumulation of a targeted compound. Moreover, these data demonstrate similar effects in different systems, including the MDA-MB-231 xenograft mouse of FIG. 10 and the KB xenograft mouse of FIG. 12.

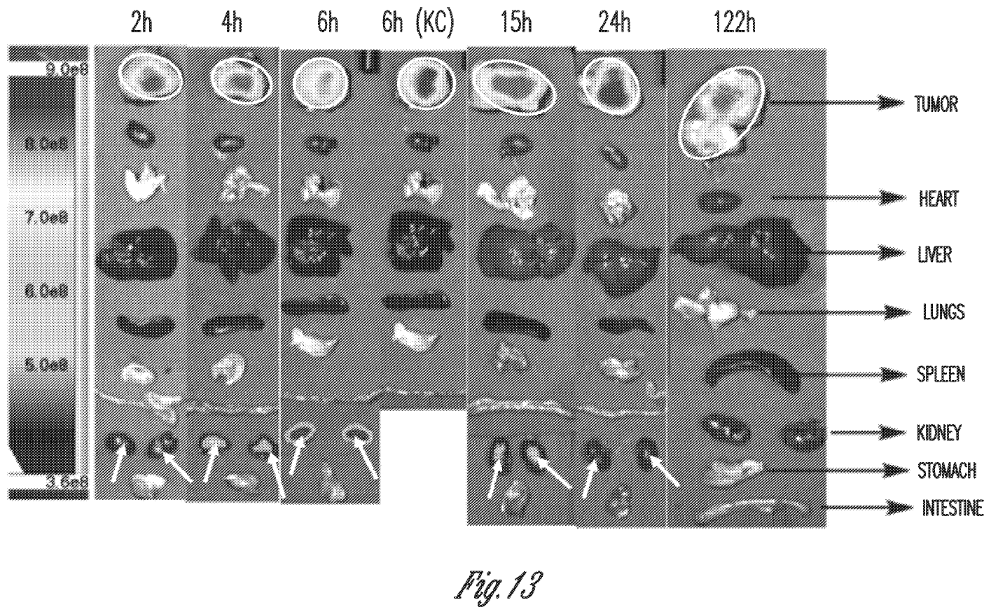

FIG. 13 shows the biodistribution (in KB tumor-bearing mice) of a targeting ligand (injected in the tail vein at a dose of 10 nmol) in the tumor, heart, liver, lung, spleen, kidney, intestine, and stomach at 2 h, 4 h, 6 h, 6 h (kidney covered (KC)), 15 h, 24 h, and 122 hours. Black or white arrows, ovals, or circles in the images highlight where the targeting ligand is present. Darker shading adjacent to an arrowhead or within an oval or circle represents a higher concentration of targeting ligand than lighter shading.

FIG. 14A shows the in vivo imaging (whole body distribution) of a targeting ligand (e.g., at a dose of 5 nmol on FADu xenograft mice (e.g., M1, M2, and M3) 6 hours post-injection. The biodistribution in the tumor, heart, liver, lung, spleen, kidney, intestine, muscle, and stomach after 6 hours is shown in FIG. 14B. FIG. 14C shows the in vivo imaging (whole-body distribution) of a competition experiment between a targeting ligand (e.g., at a dose of 5 nmol) and a unlabelled competitor 500 nmol on FADu xenograft mice (e.g., M1, M2, and M3) 6 hours post-injection. The biodistribution in the tumor, heart, liver, lung, spleen, kidney, intestine, muscle, and stomach after 6 hours is shown in FIG. 14D. Black or white arrows, ovals, or circles in the images highlight where the targeting ligand is present. Darker shading adjacent to an arrowhead or within an oval or circle represents a higher concentration of targeting ligand than lighter shading. As can be seen in FIG. 14A and FIG. 14B, a FAP-targeting compound targets the tumor, with little off-target accumulation. FIG. 14C and FIG. 14D demonstrate that in the presence of a FAP-targeting competitor, there is less accumulation of the FAP-targeting compound at the tumor (e.g., due to the competition for FAP) and FIG. 14D demonstrates that there are more off-target effects (e.g., in the stomach and kidneys) when there is competition for the FAP.

Further, as can be seen in FIG. 13 and FIG. 14B, high concentrations of active agent are seen at the tumor location for 5 days, or more. By contrast, other organs are demonstrably free of FAP-targeting compound. There does appear to be some accumulation of active agent in the kidneys, but such accumulation appears to peak around 6 hours, with a significant decrease in concentration in the kidneys observed by 15 hours, and near complete clearance by 24 hours. In some instances, use of targeting ligands to deliver active payloads facilitates good delivery of a payload to a target location with good (e.g., little or no) off-target effects or side effects. Moreover, in some instances, the ability to maintain delivery at the target location for days with a single administration facilitates less frequent therapeutic administration, improved patient compliance (e.g., through less frequent administration requirements), decreased side effects (e.g., less frequent administration further reduces off target/side effects), and/or other benefits.

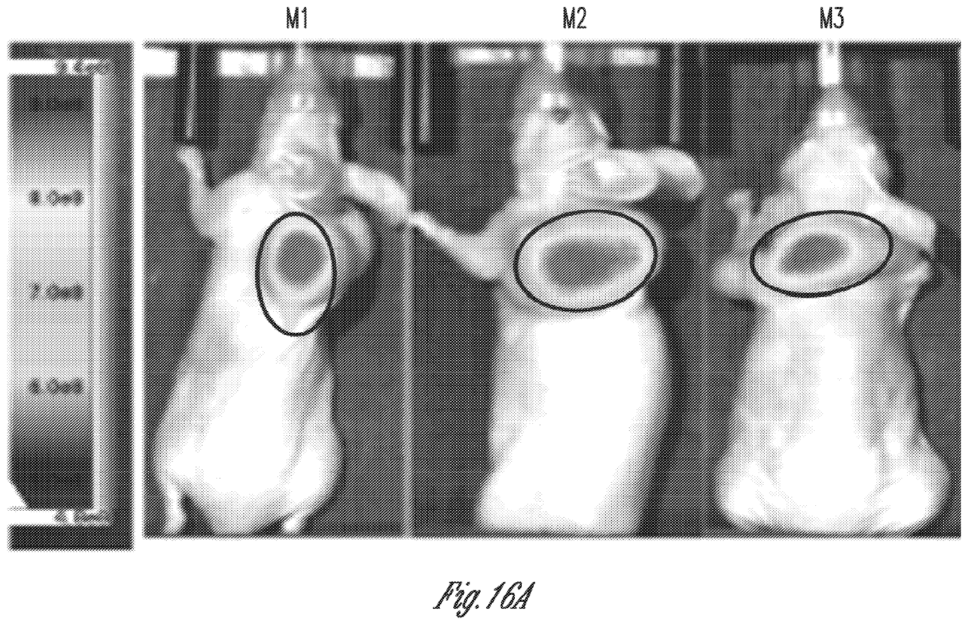

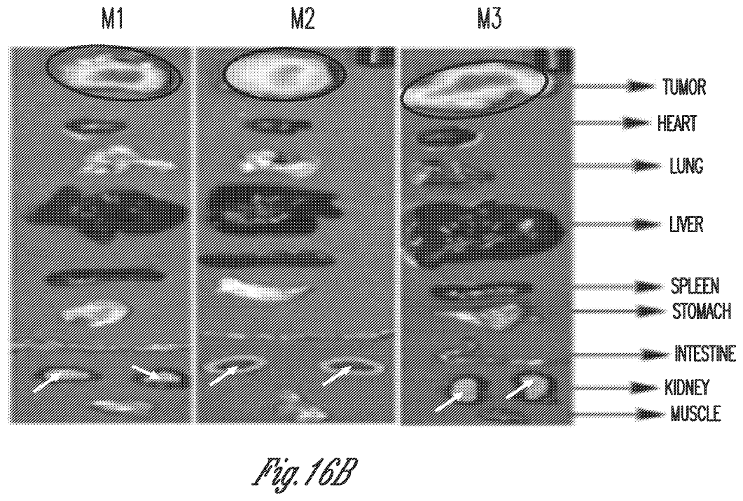

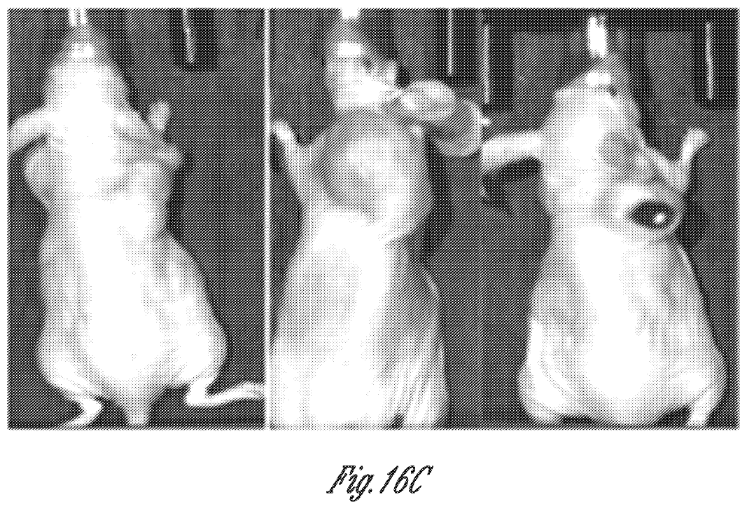

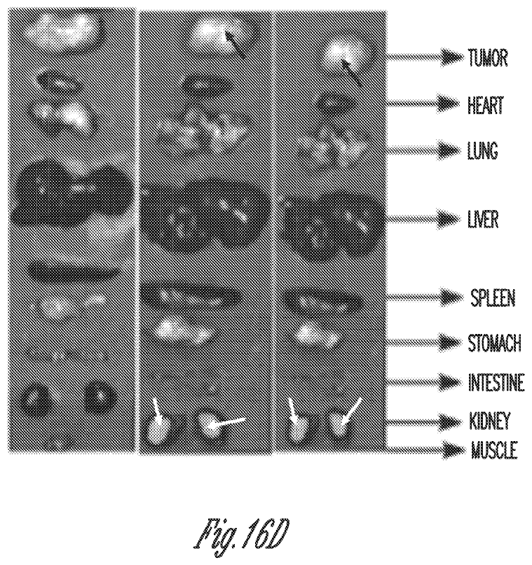

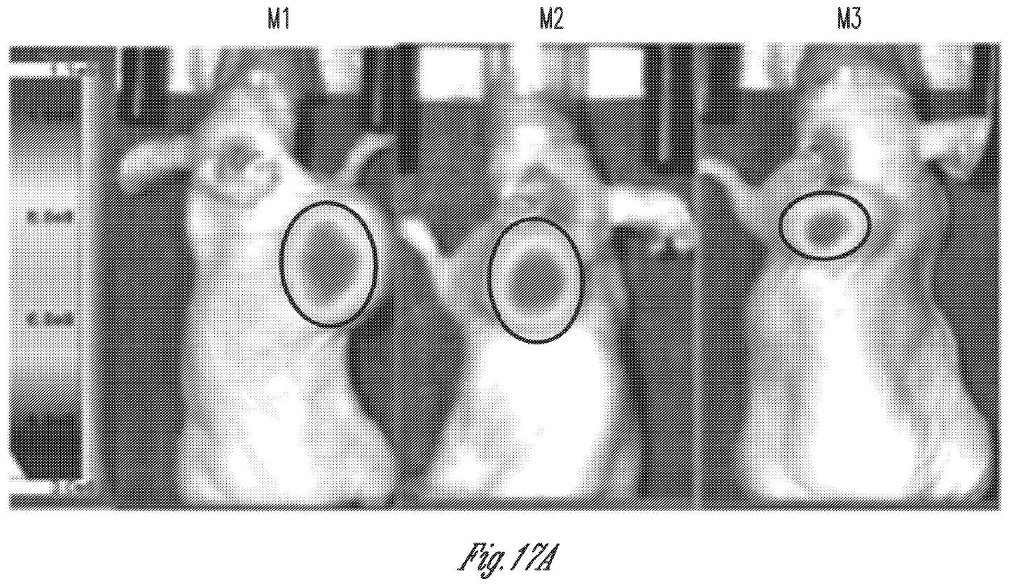

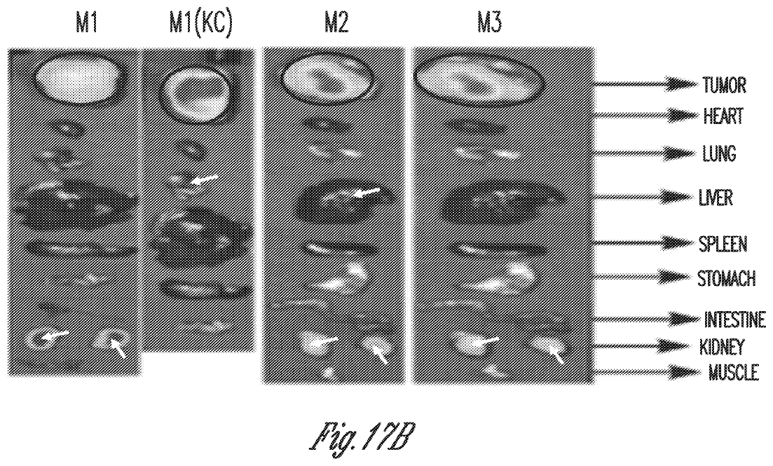

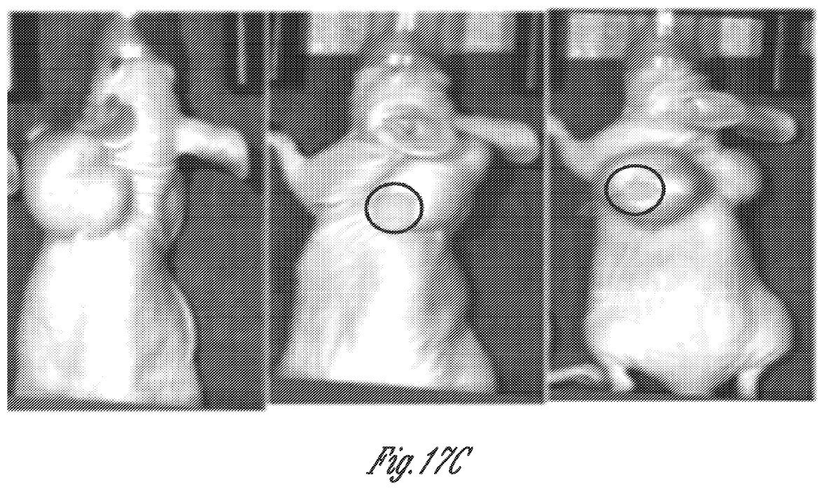

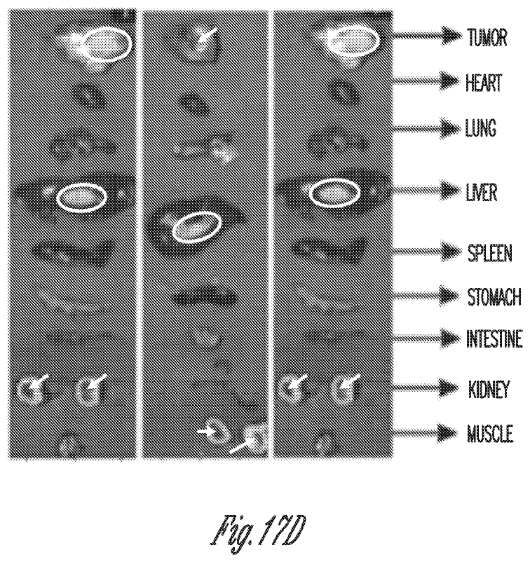

FIG. 15A shows the in vivo imaging (whole-body distribution) of a targeting ligand (5 nmol) on HT29 xenograft mice (e.g., M1, M2, and M3) 6 hours post-injection. The biodistribution in the tumor, heart, liver, lung, spleen, kidney, intestine, muscle, and stomach after 6 hours is shown in FIG. 15B. FIG. 15C shows the in vivo imaging (whole-body distribution) of a competition experiment between a targeting ligand (5 nmol) and an unlabelled competitor (500 nmol) on HT29 xenograft mice (e.g., M1, M2, and M3) 6 hours post-injection. The biodistribution in the tumor, heart, liver, lung, spleen, kidney, intestine, muscle, and stomach after 6 hours is shown in FIG. 15D. Black or white arrows, ovals, or circles in the images highlight where the targeting ligand is present. Darker shading adjacent to an arrowhead or within an oval or circle represents a higher concentration of targeting ligand than lighter shading. FIG. 16A shows the in vivo imaging (whole body distribution) of a targeting ligand (5 nmol) on KB tumor xenograft mice (e.g., M1, M2, and M3) 6 hours post-injection. The biodistribution in the tumor, heart, liver, lung, spleen, kidney, intestine, muscle, and stomach after 6 hours is shown in FIG. 16B. FIG. 16C shows the in vivo imaging (whole-body distribution) of a competition experiment between a targeting ligand (5 nmol) and a unlabelled competitor (500 nmol) on KB tumor xenograft mice (e.g., M1, M2, and M3) 6 hours post-injection. The biodistribution in the tumor, heart, liver, lung, spleen, kidney, intestine, muscle, and stomach after 6 hours is shown in FIG. 16D. Black or white arrows, ovals, or circles in the images highlight where the targeting ligand is present. Darker shading adjacent to an arrowhead or within an oval or circle represents a higher concentration of targeting ligand than lighter shading. FIG. 17A shows the in vivo imaging (whole body distribution) of a targeting ligand provided herein (e.g., at a concentration of 5 nmol) on MDA-MB-231 tumor xenograft mice (e.g., M1, M2, and M3) 6 hours post-injection. The biodistribution in the tumor, heart, liver, lung, spleen, kidney, intestine, muscle, and stomach (e.g., for M1, M1 kidney covered (KC), M2, and M3) after 6 hours is shown in FIG. 17B. FIG. 17C shows the in vivo imaging (whole-body distribution) of a competition experiment between a targeting ligand (e.g., at a concentration of 5 nmol) and a unlabelled competitor (500 nmol) on MDA-MB-231 tumor mice (e.g., M1, M2, and M3) 6 hours post-injection. The biodistribution in the tumor, heart, liver, lung, spleen, kidney, intestine, muscle, and stomach after 6 hours is shown in FIG. 17D. Black or white arrows, ovals, or circles in the images highlight where the targeting ligand is present. Darker shading adjacent to an arrowhead or within an oval or circle represents a higher concentration of targeting ligand than lighter shading. FIG. 18A shows the in vivo imaging (whole-body distribution) of a targeting ligand (5 nmol) on U87MG tumor xenograft mice (e.g., M1, M2, and M3) 6 hours post-injection. The biodistribution in the tumor, heart, liver, lung, spleen, kidney, intestine, muscle, and stomach (e.g., for M1, M1 kidney covered (KC), M2, M3 KC, and M3) after 6 hours is shown in FIG. 18B. FIG. 18C shows the in vivo imaging (whole-body distribution) of a competition experiment between a targeting ligand (5 nmol) and a unlabelled competitor (500 nmol) on U87MG tumor mice (e.g., M1, M2, and M3) 6 hours post-injection. The biodistribution in the tumor, heart, liver, lung, spleen, kidney, intestine, muscle, and stomach after 6 hours is shown in FIG. 18D. Black or white arrows, ovals, or circles in the images highlight where the targeting ligand is present. Darker shading adjacent to an arrowhead or within an oval or circle represents a higher concentration of targeting ligand than lighter shading. FIG. 19A shows the in vivo imaging (whole-body distribution) of a targeting ligand provided herein (e.g., at a dose of 5 nmol) on PANC1 tumor xenograft mice (e.g., M1, M2, and M3) 6 hours post-injection. The biodistribution in the tumor, heart, liver, lung, spleen, kidney, intestine, muscle, and stomach (e.g., for M1, M1 kidney covered (KC), M2 KC, M2, M3 KC, and M3) after 6 hours is shown in FIG. 18B. FIG. 18C shows the in vivo imaging (whole-body distribution) of a competition experiment between a targeting ligand (e.g., at a dose of 5 nmol) and a unlabelled competitor (500 nmol) on PANC1 tumor mice (e.g., M1, M2, and M3) 6 hours post-injection. The biodistribution in the tumor, heart, liver, lung, spleen, kidney, intestine, muscle, and stomach after 6 hours is shown in FIG. 18D. Black or white arrows, ovals, or circles in the images highlight where the targeting ligand is present. Darker shading adjacent to an arrowhead or within an oval or circle represents a higher concentration of targeting ligand than lighter shading. FIG. 20A shows the in vivo imaging (whole-body distribution) of a targeting ligand (e.g., at a dose of 5 nmol) only vs. targeting ligand and an unlabelled competitor) on 4T1 tumor xenograft mice 2 hours post-injection. FIG. 20B shows the in vivo imaging (whole-body distribution) of a targeting ligand (e.g., at a dose of 5 Nmol) only vs. targeting ligand and an unlabelled competitor) on 4T1 tumor xenograft mice 6 hours post-injection. The biodistribution in the tumor, heart, liver, lung, spleen, kidney, intestine, muscle, and stomach (e.g., targeted, targeted kidneys covered (KC), and competition) after 6 hours is shown in FIG. 20C. Black or white arrows, ovals, or circles in the images highlight where the targeting ligand is present. Darker shading adjacent to an arrowhead or within an oval or circle represents a higher concentration of targeting ligand than lighter shading. These results are consistent with results presented in other figures, further demonstrating the consistency of results for various tumor types and/or models.

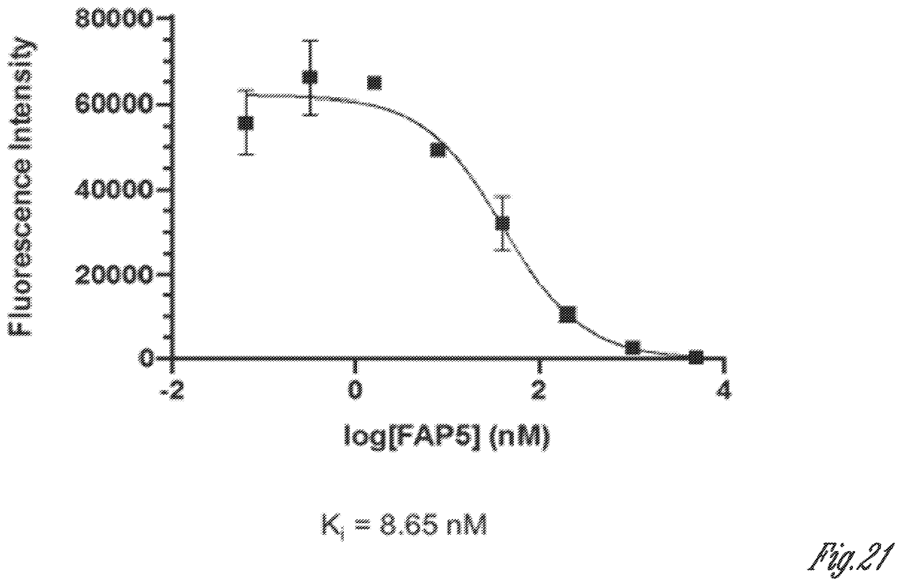

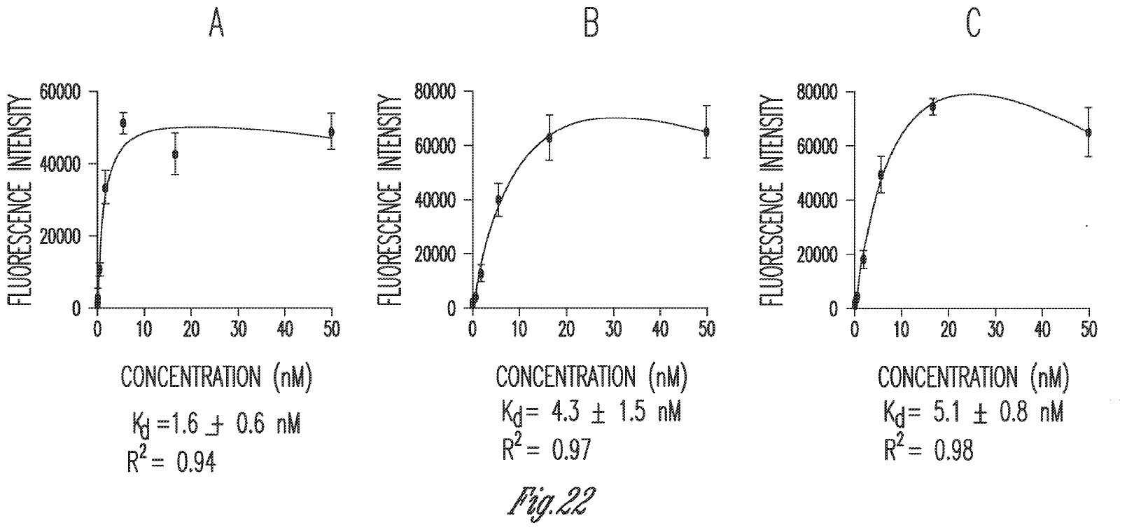

In addition, FIGS. 21-22 show that several compounds (e.g., compound 21, compound 1, compound 5, and compound 6) target cells (with a very high affinity (e.g., from 1 nM to 10 nM)) that express FAP (e.g., FAP5).

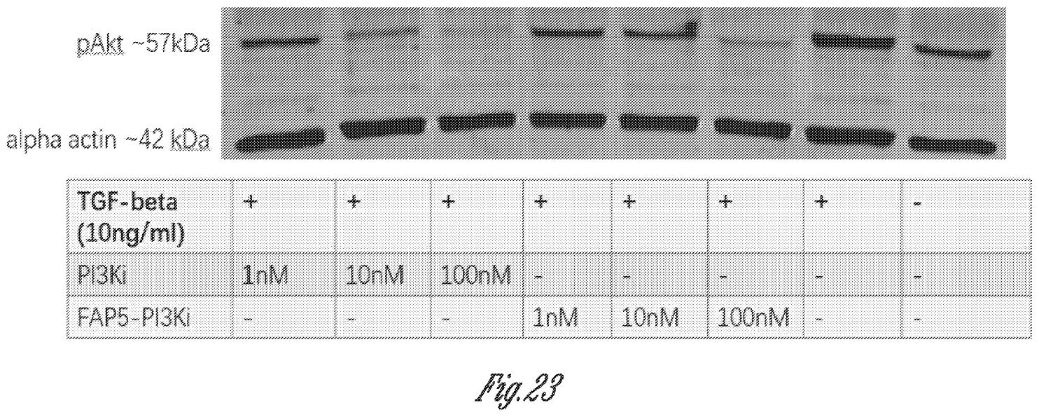

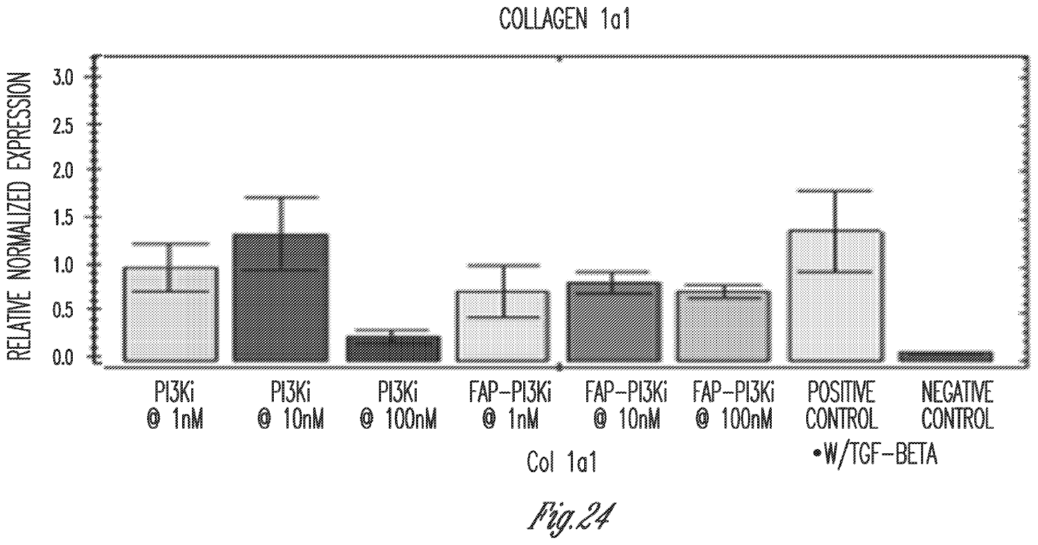

Furthermore, in conjunction with FIGS. 9A-22, showing that a compound localizes to a target site, FIG. 23 and FIG. 24 show that a compound (e.g., compound 11) has efficacy (e.g., reduces fibrotic response) against FAP (e.g., FAP5) expressing cells. For example, a compound (e.g., compound 11) has comparable efficacy to a PI3Ki alone at reducing pathological biological responses (e.g., reducing phosphorylation of Akt in TGF-β-stimulated human lung fibroblast cells (FIG. 23) and reducing relative expression of collagen 1A1 mRNA in TGF-β-stimulated human lung fibroblast cells (FIG. 24). So, while a payload (e.g., PI3Ki) alone effectively reduces pathogenic biological responses, a compound provided herein effectively reduces a pathogenic biological response and increases residence time at a target site (e.g., a tumor or fibrotic tissue), ultimately increasing the effective concentration of the payload at the target site (e.g., reducing side effects, decreasing dosing, and the like).

B can be an imaging agent. B can be a radio-imaging agent. B can be a photodynamic therapeutic agent. B can be a chemotherapeutic agent. B can be an antifibrotic agent. B can be a radiotherapeutic agent. B can be an anticancer agent. B can be anticancer agent effective against cancer cells or cancer-associated fibroblasts, myofibroblasts, or other tumor microenvironment factor.

B can comprise a radio-imaging nuclide. The radio-imaging nuclide can be any suitable radio-imaging nuclide. The radio-imaging nuclide can be selected from the group consisting of ^{99m}Tc, ¹¹¹In, ¹⁸F, ⁶⁸Ga, ¹²⁴I, ¹²⁵I, and ¹³¹I.

B can comprise a radiotherapeutic nuclide. The radiotherapeutic nuclide can be selected from the group consisting of ¹⁷⁷Lu, ⁹⁰Y, and ²¹¹At.

B can be a chelator, and that in the case of radiotherapeutic nuclides B can chelate the nuclide.

B can comprise a radiolabelled prosthetic group (or a radical thereof). The radiolabelled prosthetic group can comprise a radioisotope selected from the group consisting of ¹⁸F, ¹²⁴I, ¹²⁵I, ¹³¹I, and ²¹¹At.

A-L- has the following structure:

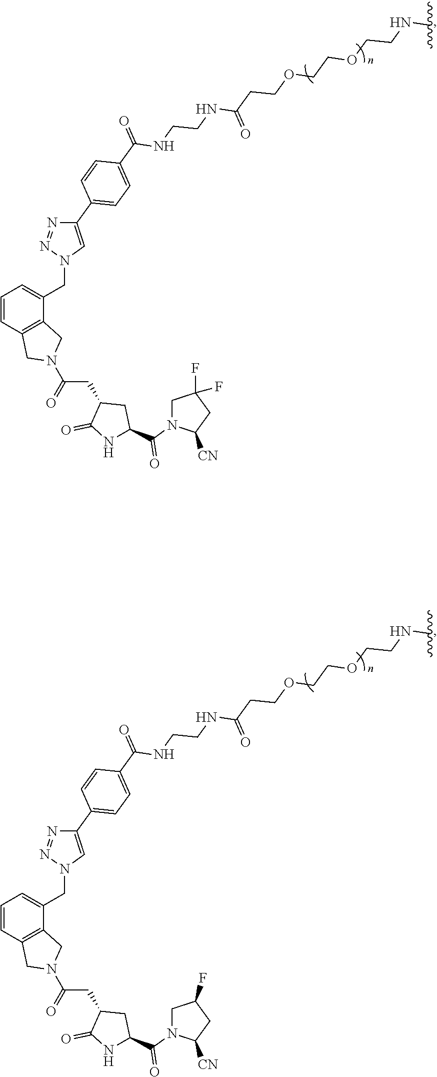

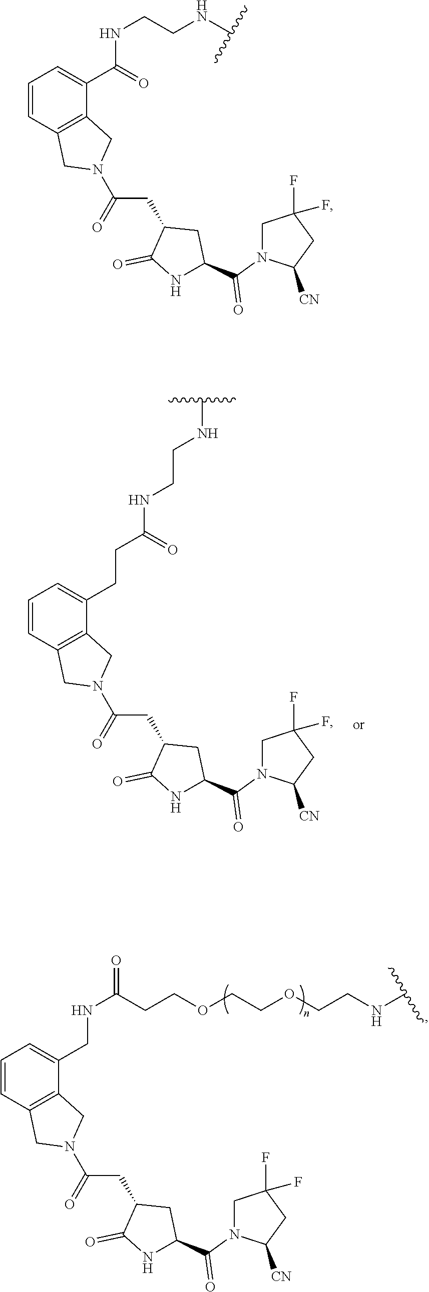

Wherein n is an inter from 1-5.

B (e.g., the radiolabelled prosthetic group (or a radical thereof)) has the following structure:

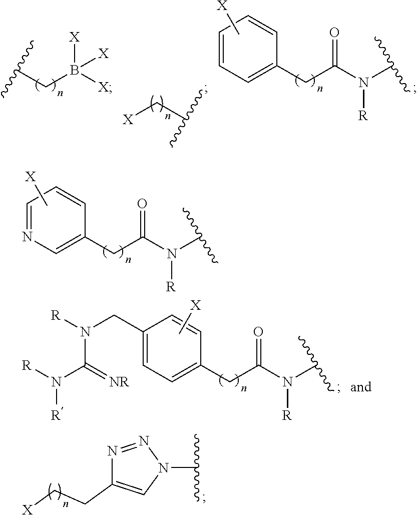

Wherein:
each X is independently a radioisotope selected from the group consisting of ¹⁸F, ¹²⁴I, ¹²⁵I, ¹³¹I, and ²¹¹At;
each R or R¹ is independently H, alkyl, substituted alkyl, cycloalkyl, substituted cycloalkyl, heterocycloalkyl, aryl, substituted aryl, heteroaryl, or substituted heteroaryl; and
each n is independently an integer selected from the group consisting of 0, 1, 2, 3, 4, 5, 6, 7, 8, 9, 10, 11, 12, 13, 14, 15, 16, 17, 18, 19, and 20.

Representative radiolabelled prosthetic groups (e.g., B) include, but are not limited to:

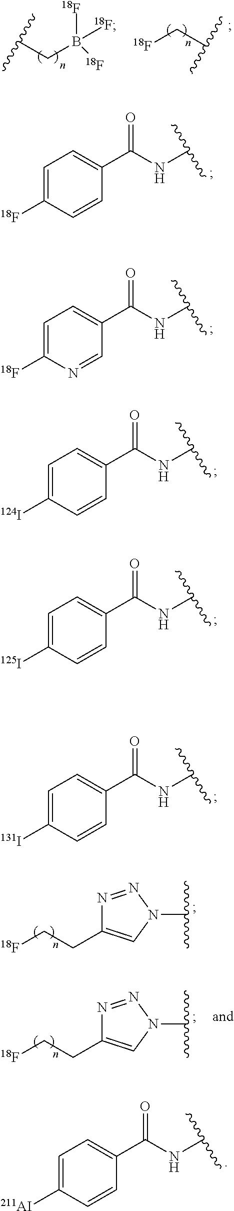

B can be a chelating group (e.g., a chelating agent (or a radical thereof)). Representative chelating groups include, but are not limited to (including free bases thereof, such as wherein a proton (H+) of one or more CO₂H (COOH) is removed to form COO—):

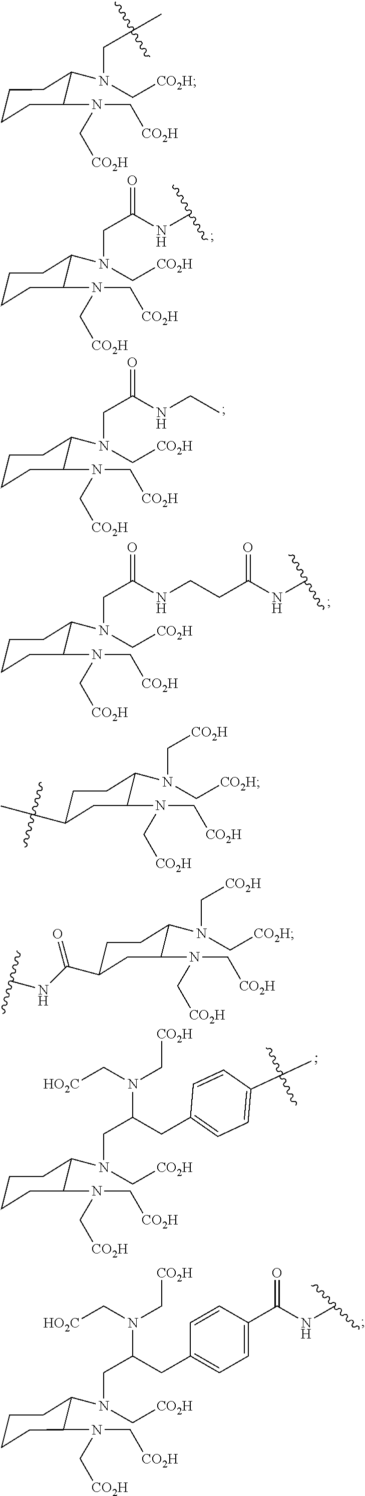

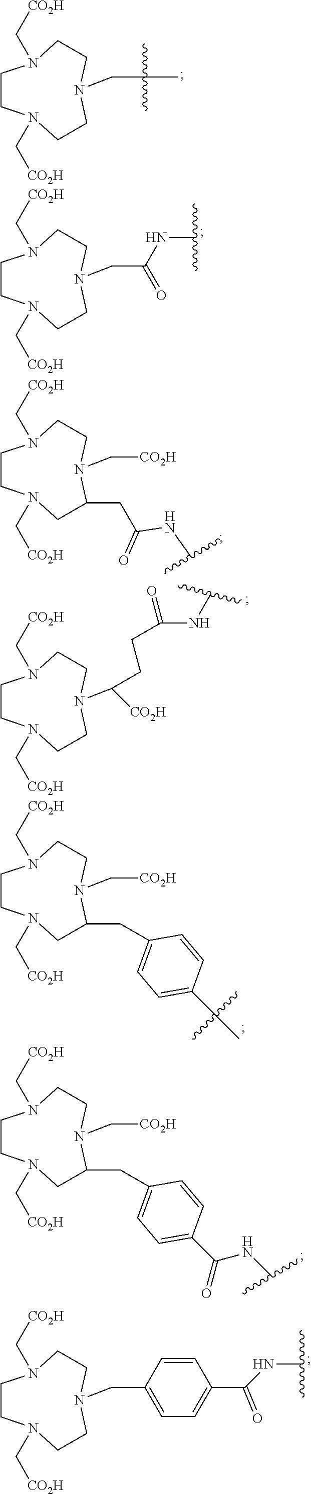

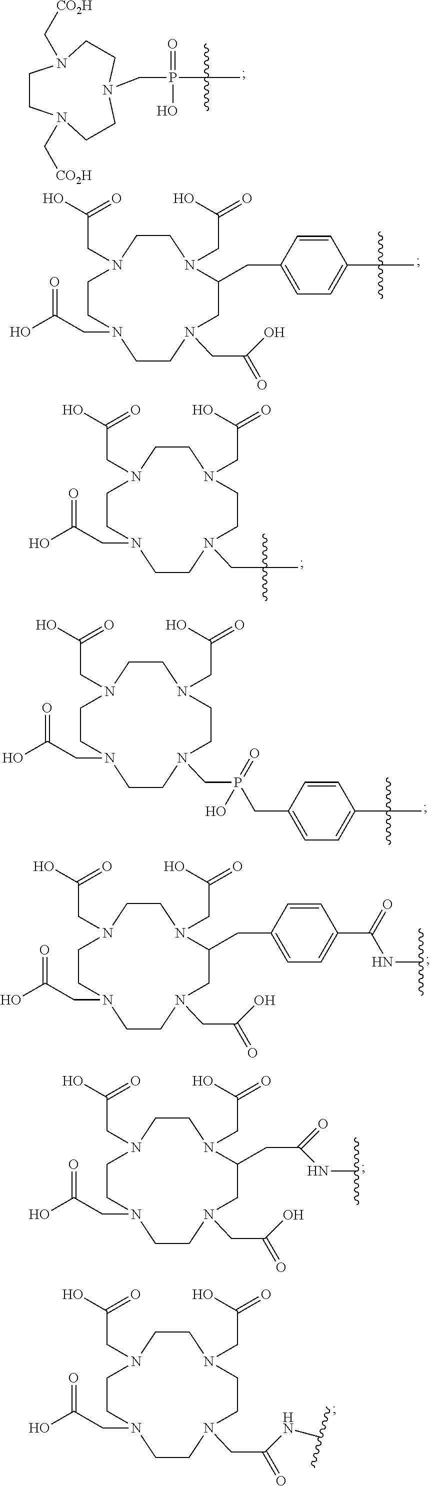

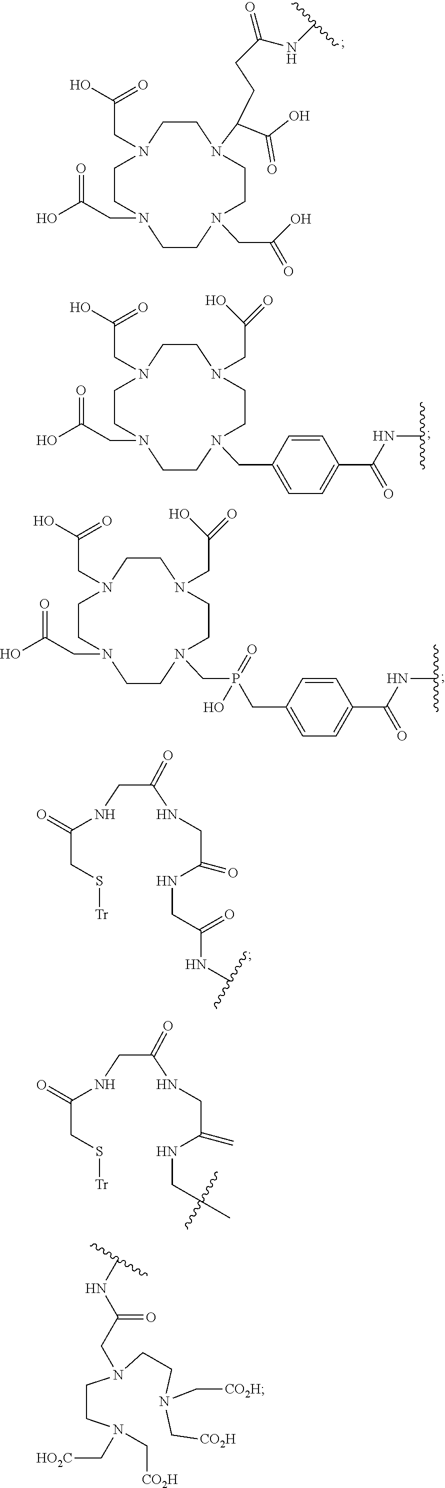

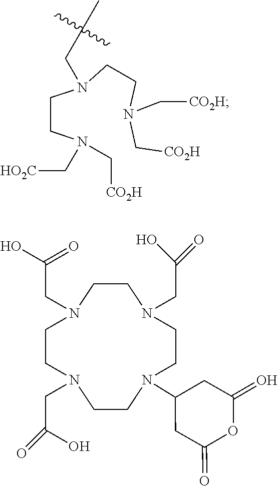 [SMILES: *CN(CCN(CC(=O)O)CC(=O)O)CCN(CC(=O)O)CC(=O)O ; O=C(O)CN1CCN(CC(=O)O)CCN(C2CC(=O)OC(=O)C2)CCN(CC(=O)O)CC1]

B can be an antifibrotic agent or a radical thereof.

B can be a PI-3 kinase inhibitor or a radical thereof.

B can be a transforming growth factor beta (TGFβ)/Smad inhibitor or a radical thereof.

B can be a Wingless-related integration site (Wnt)/β-catenin inhibitor or a radical thereof.

B can be a kinase inhibitor, or a radical thereof, for vascular endothelial growth factor receptor (VEGFR1, VEGFR2, VEGFR3), fibroblast growth factor receptor (FGFR1 or FGFR2), or platelet-derived growth factor receptor (PDGFR).

B can be a kinase inhibitor for focal adhesion kinase (FAK) or Rho-associated protein kinase (ROCK), or a radical thereof.

B can an agonist of a toll-like receptor (TLR), or a radical thereof.

B can be an inhibitor of NF-κB (nuclear factor kappa-light-chain-enhancer of activated B cells), or a radical thereof.

B can be an inhibitor of collagen synthesis, or a radical thereof.

B is attached to L via a hydroxyl radical.

A PI-3 Kinase inhibitor (or a radical thereof) (e.g., a compound or a conjugate comprising a PI-3 Kinase inhibitor (or a radical thereof)) can have the structure of Formula III:

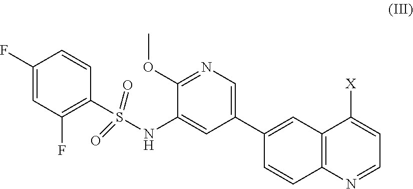

wherein:
X is selected from the group consisting of:

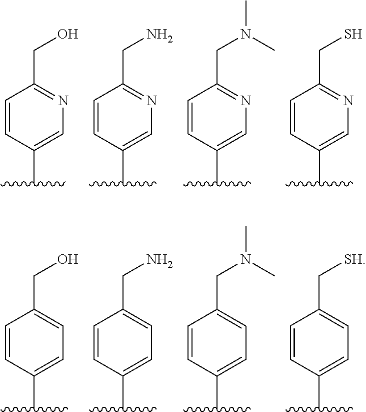

X can be the radical of B (e.g., wherein the radical is on a heteroatom (e.g., S, N, or O of X)). B can be attached to L via X (e.g., a hydroxyl radical of X).

A compound (e.g., conjugate) can have the following structure:

TABLE 2

| A | L | B |
|---|---|---|
| 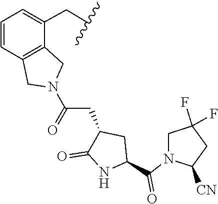 [SMILES: *Cc1cccc2c1CN(C(=O)C[C@@H]1C[C@@H](C(=O)N3CC(F)(F)C[C@H]3C#N)NC1=O)C2] | 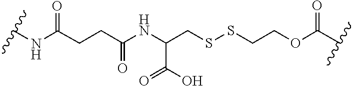 [SMILES: *NC(=O)CCC(=O)NC(CSSCCOC(*)=O)C(=O)O] | 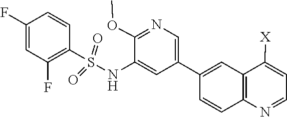 |
| 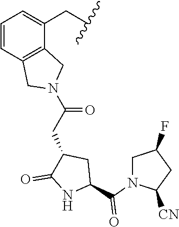 [SMILES: *Cc1cccc2c1CN(C(=O)C[C@@H]1C[C@@H](C(=O)N3C[C@@H](F)C[C@H]3C#N)NC1=O)C2] | 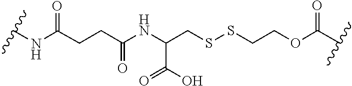 [SMILES: *NC(=O)CCC(=O)NC(CSSCCOC(*)=O)C(=O)O] | 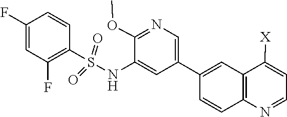 |

wherein:
X is selected from the group consisting of:

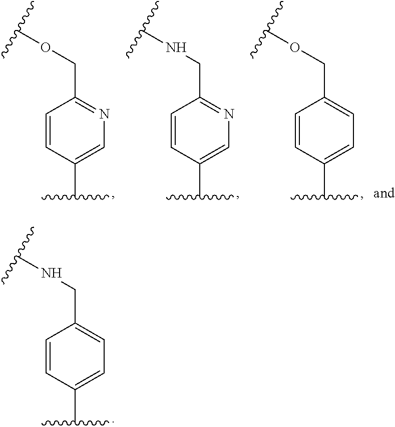 [SMILES: *OCc1ccc(*)cn1 ; *NCc1ccc(*)cn1 ; *OCc1ccc(*)cc1 ; *NCc1ccc(*)cc1]

A PI-3 Kinase inhibitor (or a radical thereof) (e.g., a compound or a conjugate comprising a PI-3 Kinase inhibitor (or a radical thereof)) can have the structure of:

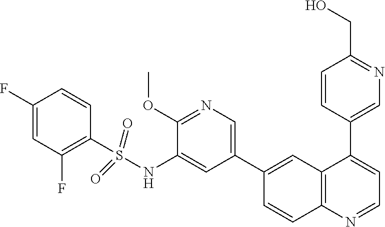 [SMILES: COc1ncc(-c2ccc3nccc(-c4ccc(CO)nc4)c3c2)cc1NS(=O)(=O)c1ccc(F)cc1F]

A compound (e.g., conjugate) can have the following structure: Table 3 Example A L

TABLE 3

| Example | A | L | B |
|---|---|---|---|
| 11 | 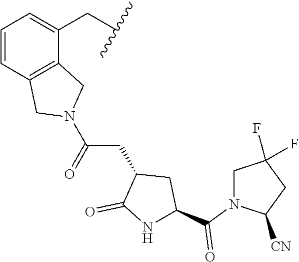 [SMILES: *Cc1cccc2c1CN(C(=O)C[C@@H]1C[C@@H](C(=O)N3CC(F)(F)C[C@H]3C#N)NC1=O)C2] | 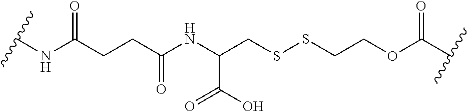 [SMILES: *NC(=O)CCC(=O)NC(CSSCCOC(*)=O)C(=O)O] | 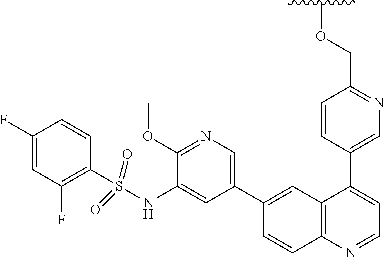 [SMILES: *OCc1ccc(-c2ccnc3ccc(-c4cnc(OC)c(NS(=O)(=O)c5ccc(F)cc5F)c4)cc23)cn1] |
| 12 | 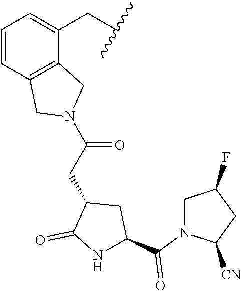 [SMILES: *Cc1cccc2c1CN(C(=O)C[C@@H]1C[C@@H](C(=O)N3C[C@@H](F)C[C@H]3C#N)NC1=O)C2] | 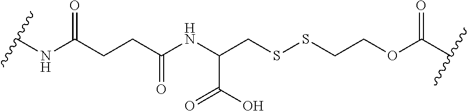 [SMILES: *NC(=O)CCC(=O)NC(CSSCCOC(*)=O)C(=O)O] | 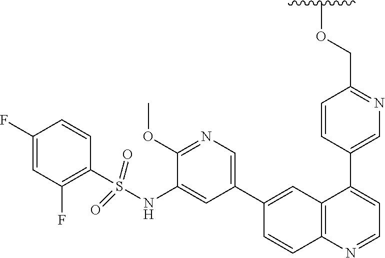 [SMILES: *OCc1ccc(-c2ccnc3ccc(-c4cnc(OC)c(NS(=O)(=O)c5ccc(F)cc5F)c4)cc23)cn1] |
| 13 | 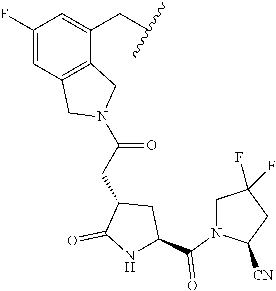 [SMILES: *Cc1cc(F)cc2c1CN(C(=O)C[C@@H]1C[C@@H](C(=O)N3CC(F)(F)C[C@H]3C#N)NC1=O)C2] | 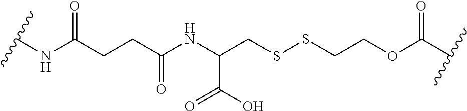 [SMILES: *NC(=O)CCC(=O)NC(CSSCCOC(*)=O)C(=O)O] | 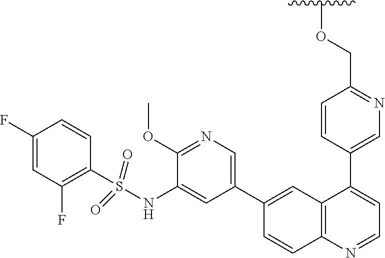 [SMILES: *OCc1ccc(-c2ccnc3ccc(-c4cnc(OC)c(NS(=O)(=O)c5ccc(F)cc5F)c4)cc23)cn1] |
| 14 | 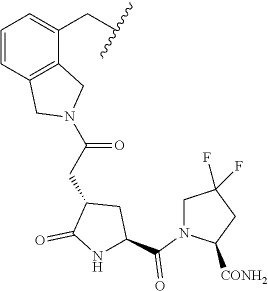 [SMILES: *Cc1cccc2c1CN(C(=O)C[C@@H]1C[C@@H](C(=O)N3CC(F)(F)C[C@H]3C(N)=O)NC1=O)C2] | 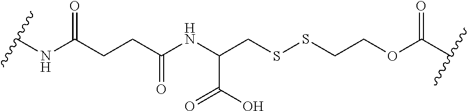 [SMILES: *NC(=O)CCC(=O)NC(CSSCCOC(*)=O)C(=O)O] | 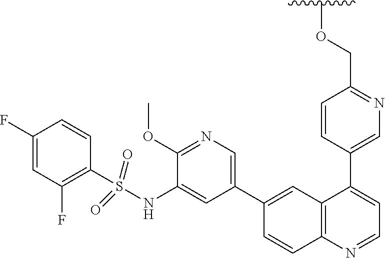 [SMILES: *OCc1ccc(-c2ccnc3ccc(-c4cnc(OC)c(NS(=O)(=O)c5ccc(F)cc5F)c4)cc23)cn1] |
| 15 | 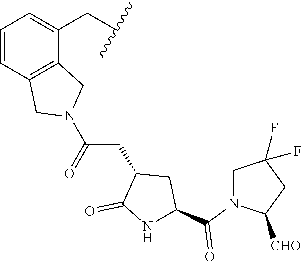 [SMILES: *Cc1cccc2c1CN(C(=O)C[C@@H]1C[C@@H](C(=O)N3CC(F)(F)C[C@H]3C=O)NC1=O)C2] | 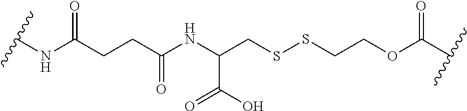 [SMILES: *NC(=O)CCC(=O)NC(CSSCCOC(*)=O)C(=O)O] | 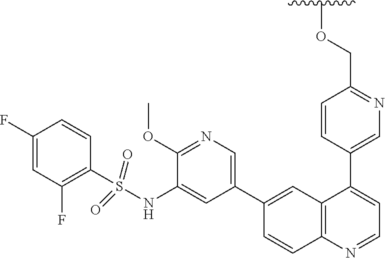 [SMILES: *OCc1ccc(-c2ccnc3ccc(-c4cnc(OC)c(NS(=O)(=O)c5ccc(F)cc5F)c4)cc23)cn1] |
| 16 | 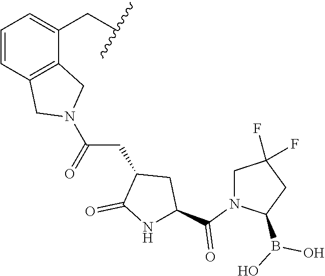 [SMILES: *Cc1cccc2c1CN(C(=O)C[C@@H]1C[C@@H](C(=O)N3CC(F)(F)C[C@H]3B(O)O)NC1=O)C2] | 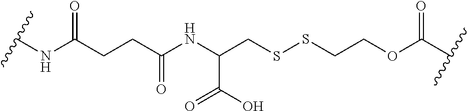 [SMILES: *NC(=O)CCC(=O)NC(CSSCCOC(*)=O)C(=O)O] | 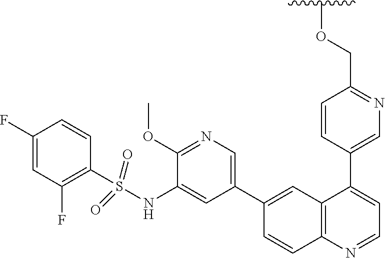 [SMILES: *OCc1ccc(-c2ccnc3ccc(-c4cnc(OC)c(NS(=O)(=O)c5ccc(F)cc5F)c4)cc23)cn1] |
| 17 | 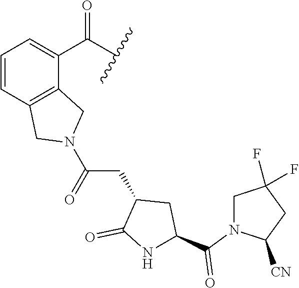 [SMILES: *C(=O)c1cccc2c1CN(C(=O)C[C@@H]1C[C@@H](C(=O)N3CC(F)(F)C[C@H]3C#N)NC1=O)C2] | 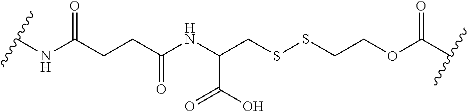 [SMILES: *NC(=O)CCC(=O)NC(CSSCCOC(*)=O)C(=O)O] | 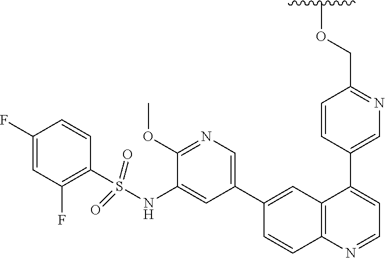 [SMILES: *OCc1ccc(-c2ccnc3ccc(-c4cnc(OC)c(NS(=O)(=O)c5ccc(F)cc5F)c4)cc23)cn1] |
| 18 | 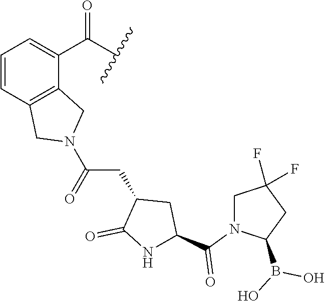 [SMILES: *C(=O)c1cccc2c1CN(C(=O)C[C@@H]1C[C@@H](C(=O)N3CC(F)(F)C[C@H]3B(O)O)NC1=O)C2] | 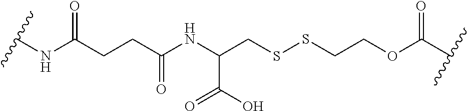 [SMILES: *NC(=O)CCC(=O)NC(CSSCCOC(*)=O)C(=O)O] | 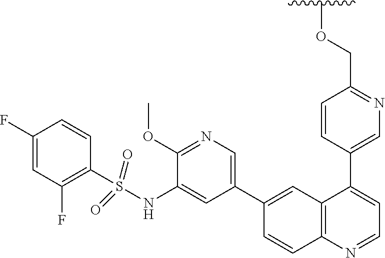 [SMILES: *OCc1ccc(-c2ccnc3ccc(-c4cnc(OC)c(NS(=O)(=O)c5ccc(F)cc5F)c4)cc23)cn1] |
| 19 | 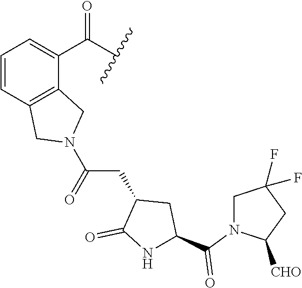 [SMILES: *C(=O)c1cccc2c1CN(C(=O)C[C@@H]1C[C@@H](C(=O)N3CC(F)(F)C[C@H]3C=O)NC1=O)C2] | 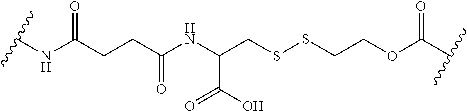 [SMILES: *NC(=O)CCC(=O)NC(CSSCCOC(*)=O)C(=O)O] | 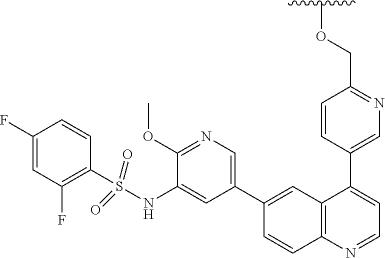 [SMILES: *OCc1ccc(-c2ccnc3ccc(-c4cnc(OC)c(NS(=O)(=O)c5ccc(F)cc5F)c4)cc23)cn1] |

A compound (e.g., conjugate) can have the following structure:

TABLE 4

| Compound | Structure |
|---|---|
| 20 | 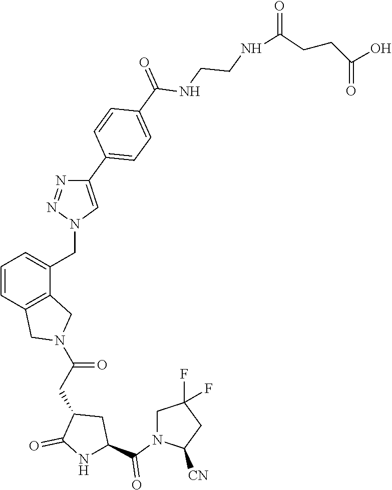 [SMILES: N#C[C@@H]1CC(F)(F)CN1C(=O)[C@@H]1C[C@@H](CC(=O)N2Cc3cccc(Cn4cc(-c5ccc(C(=O)NCCNC(=O)CCC(=O)O)cc5)nn4)c3C2)C(=O)N1] |
| 21 | 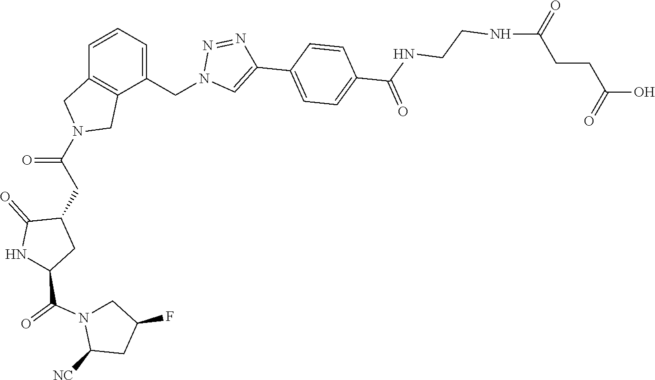 [SMILES: N#C[C@@H]1C[C@H](F)CN1C(=O)[C@@H]1C[C@@H](CC(=O)N2Cc3cccc(Cn4cc(-c5ccc(C(=O)NCCNC(=O)CCC(=O)O)cc5)nn4)c3C2)C(=O)N1] |

A compound (e.g., conjugate) can have the following structure:

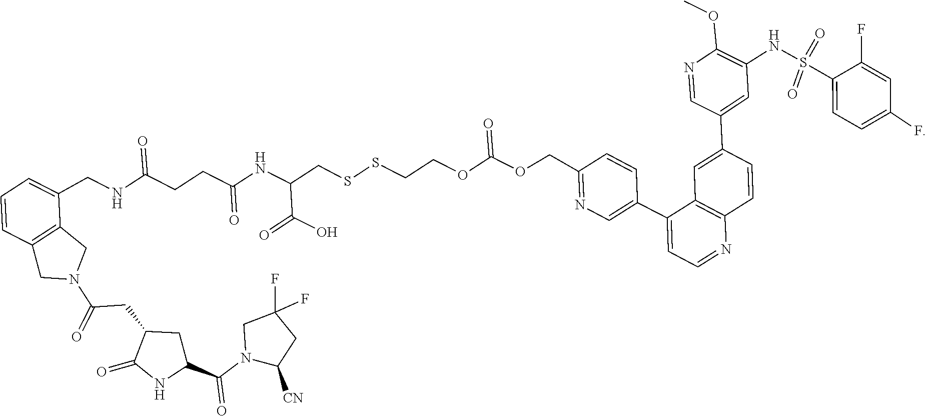

A compound (e.g., conjugate) can have the following structure:

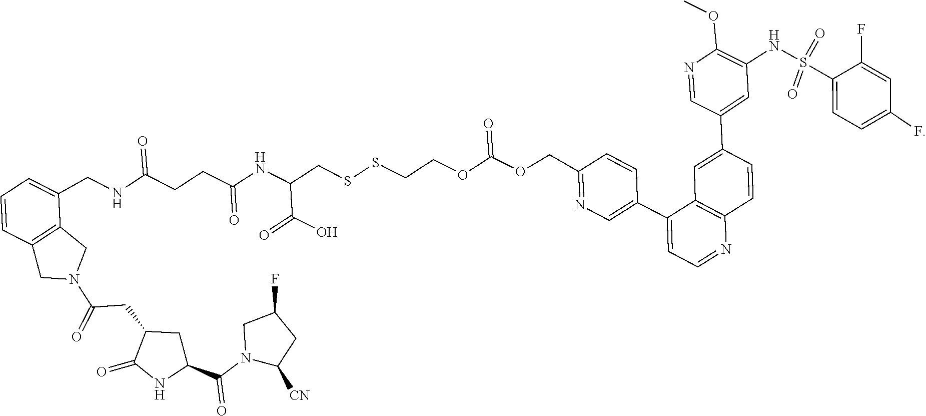 [SMILES: COc1ncc(-c2ccc3nccc(-c4ccc(COC(=O)OCCSSCC(NC(=O)CCC(=O)NCc5cccc6c5CN(C(=O)C[C@@H]5C[C@@H](C(=O)N7C[C@@H](F)C[C@H]7C#N)NC5=O)C6)C(=O)O)nc4)c3c2)cc1NS(=O)(=O)c1ccc(F)cc1F]

### Methods of Treatment

A method for treating an inflammatory disease or disorder is also provided. The method for treating an inflammatory disease or disorder by modulating the activity of activated fibroblasts. The method can comprise administering a compound (e.g., a conjugate) of any formula provided herein (e.g., Formula (I), Formula (I-A), Formula (I-B), Formula (I-C), Formula (II), Formula (III), Formula (X), Formula (X-A), Formula (X-B), Table 2, Table 3, or Table 4).

A method for treating cancer is provided. The method of treating cancer can be by modulating the activity of activated fibroblasts. The method can comprise a compound (e.g., a conjugate) of any formula provided herein (e.g., Formula (I), Formula (I-A), Formula (I-B), Formula (I-C), Formula (II), Formula (III), Formula (X), Formula (X-A), Formula (X-B), Table 2, Table 3, or Table 4). The method can comprise contacting a cancer-activated fibroblast (CAF) (e.g., a CAF of a cancer patient) with a compound (e.g., a conjugate) of any formula provided herein (e.g., Formula (I), Formula (I-A), Formula (I-B), Formula (I-C), Formula (II), Formula (III), Formula (X), Formula (X-A), Formula (X-B), Table 2, Table 3, or Table 4).

A method for treating fibrosis is also provided. The method of treating fibrosis can be by modulating the activity of activated fibroblasts. The method can comprise administering a compound (e.g., a conjugate) of any formula provided herein (e.g., Formula (I), Formula (I-A), Formula (I-B), Formula (I-C), Formula (II), Formula (III), Formula (X), Formula (X-A), Formula (X-B), Table 2, Table 3, or Table 4).

The method can be chemotherapy or radiotherapy.

A method for imaging cancer or fibrosis in a subject with the cancer or the fibrosis is provided.

Also provided are compositions and methods for optical imaging. The compositions and methods can be for fluorescence-guided surgery. The compositions and methods can be for radio-imaging.

The methods above comprise the steps of: providing to the patient in need thereof with a pharmaceutically effective amount of conjugate A-L-B, wherein A comprises a fibroblast activation protein alpha (FAPα) targeting moiety, with a molecular weight below 10,000; L comprises a bi-functionalized linker, which can form chemical bonds with A and B; and B comprises an optical dye (e.g., a fluorescent dye), a photodynamic therapeutic agent, a radio-imaging agent, a radiotherapeutic agent, a chemotherapeutic agent, an antifibrotic agent, or an anticancer agent that is effective against cancer cells or cancer-associated fibroblasts, myofibroblasts or other tumor microenvironment factors.

### Pharmaceutical Compositions, Routes of Administration, and Dosing

In certain embodiments, the disclosure is directed to a pharmaceutical composition, comprising a compound and a pharmaceutically acceptable carrier. In certain embodiments, the pharmaceutical composition comprises a plurality of compounds and a pharmaceutically acceptable carrier.

In certain embodiments, a pharmaceutical composition further comprises at least one additional pharmaceutically active agent. The at least one additional pharmaceutically active agent can be an agent useful in the treatment of ischemia-reperfusion injury.

Pharmaceutical compositions can be prepared by combining one or more compounds with a pharmaceutically acceptable carrier and, optionally, one or more additional pharmaceutically active agents.

As stated above, an “effective amount” refers to any amount that is sufficient to achieve a desired biological effect. Combined with the teachings provided herein, by choosing among the various active compounds and weighing factors such as potency, relative bioavailability, patient body weight, severity of adverse side-effects and mode of administration, an effective prophylactic or therapeutic treatment regimen can be planned which does not cause substantial unwanted toxicity and yet is effective to treat the particular subject. The effective amount for any particular application can vary depending on such factors as the disease or condition being treated, the particular compound being administered, the size of the subject, or the severity of the disease or condition. One of ordinary skill in the art can empirically determine the effective amount of a particular compound and/or other therapeutic agent without necessitating undue experimentation. A maximum dose may be used, that is, the highest safe dose according to some medical judgment. Multiple doses per day may be contemplated to achieve appropriate systemic levels of compounds. Appropriate systemic levels can be determined by, for example, measurement of the patient's peak or sustained plasma level of the drug. “Dose” and “dosage” are used interchangeably herein.

Generally, daily oral doses of a compound are, for human subjects, from about 0.01 milligrams/kg per day to 1,000 milligrams/kg per day. Oral doses in the range of 0.5 to 50 milligrams/kg, in one or more administrations per day, can yield therapeutic results.

Dosage may be adjusted appropriately to achieve desired drug levels, local or systemic, depending upon the mode of administration. For example, intravenous administration may vary from one order to several orders of magnitude lower dose per day. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of the compound.

For any compound the therapeutically effective amount can be initially determined from animal models. A therapeutically effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound.

Adjusting the dose to achieve maximal efficacy based on the methods described above and other methods as are well-known in the art is well within the capabilities of the ordinarily skilled artisan.

For clinical use, any compound can be administered in an amount equal or equivalent to 0.2-2,000 milligram (mg) of compound per kilogram (kg) of body weight of the subject per day. The compounds can be administered in a dose equal or equivalent to 2-2,000 mg of compound per kg body weight of the subject per day. The compounds can be administered in a dose equal or equivalent to 20-2,000 mg of compound per kg body weight of the subject per day. The compounds can be administered in a dose equal or equivalent to 50-2,000 mg of compound per kg body weight of the subject per day. The compounds can be administered in a dose equal or equivalent to 100-2,000 mg of compound per kg body weight of the subject per day. The compounds can be administered in a dose equal or equivalent to 200-2,000 mg of compound per kg body weight of the subject per day. Where a precursor or prodrug of a compound is to be administered, it is administered in an amount that is equivalent to, i.e., sufficient to deliver, the above-stated amounts of the compound.

The formulations of the compounds can be administered to human subjects in therapeutically effective amounts. Typical dose ranges are from about 0.01 microgram/kg to about 2 mg/kg of body weight per day. The dosage of drug to be administered is likely to depend on such variables as the type and extent of the disorder, the overall health status of the particular subject, the specific compound being administered, the excipients used to formulate the compound, and its route of administration. Routine experiments may be used to optimize the dose and dosing frequency for any particular compound.

The compounds can be administered at a concentration in the range from about 0.001 microgram/kg to greater than about 500 mg/kg. For example, the concentration may be 0.001 microgram/kg, 0.01 microgram/kg, 0.05 microgram/kg, 0.1 microgram/kg, 0.5 microgram/kg, 1.0 microgram/kg, 10.0 microgram/kg, 50.0 microgram/kg, 100.0 microgram/kg, 500 microgram/kg, 1.0 mg/kg, 5.0 mg/kg, 10.0 mg/kg, 15.0 mg/kg, 20.0 mg/kg, 25.0 mg/kg, 30.0 mg/kg, 35.0 mg/kg, 40.0 mg/kg, 45.0 mg/kg, 50.0 mg/kg, 60.0 mg/kg, 70.0 mg/kg, 80.0 mg/kg, 90.0 mg/kg, 100.0 mg/kg, 150.0 mg/kg, 200.0 mg/kg, 250.0 mg/kg, 300.0 mg/kg, 350.0 mg/kg, 400.0 mg/kg, 450.0 mg/kg, to greater than about 500.0 mg/kg or any incremental value thereof. It is to be understood that all values and ranges between these values and ranges are meant to be encompassed.

The compounds can be administered at a dosage in the range from about 0.2 milligram/kg/day to greater than about 100 mg/kg/day. For example, the dosage may be 0.2 mg/kg/day to 100 mg/kg/day, 0.2 mg/kg/day to 50 mg/kg/day, 0.2 mg/kg/day to 25 mg/kg/day, 0.2 mg/kg/day to 10 mg/kg/day, 0.2 mg/kg/day to 7.5 mg/kg/day, 0.2 mg/kg/day to 5 mg/kg/day, 0.25 mg/kg/day to 100 mg/kg/day, 0.25 mg/kg/day to 50 mg/kg/day, 0.25 mg/kg/day to 25 mg/kg/day, 0.25 mg/kg/day to 10 mg/kg/day, 0.25 mg/kg/day to 7.5 mg/kg/day, 0.25 mg/kg/day to 5 mg/kg/day, 0.5 mg/kg/day to 50 mg/kg/day, 0.5 mg/kg/day to 25 mg/kg/day, 0.5 mg/kg/day to 20 mg/kg/day, 0.5 mg/kg/day to 15 mg/kg/day, 0.5 mg/kg/day to 10 mg/kg/day, 0.5 mg/kg/day to 7.5 mg/kg/day, 0.5 mg/kg/day to 5 mg/kg/day, 0.75 mg/kg/day to 50 mg/kg/day, 0.75 mg/kg/day to 25 mg/kg/day, 0.75 mg/kg/day to 20 mg/kg/day, 0.75 mg/kg/day to 15 mg/kg/day, 0.75 mg/kg/day to 10 mg/kg/day, 0.75 mg/kg/day to 7.5 mg/kg/day, 0.75 mg/kg/day to 5 mg/kg/day, 1.0 mg/kg/day to 50 mg/kg/day, 1.0 mg/kg/day to 25 mg/kg/day, 1.0 mg/kg/day to 20 mg/kg/day, 1.0 mg/kg/day to 15 mg/kg/day, 1.0 mg/kg/day to 10 mg/kg/day, 1.0 mg/kg/day to 7.5 mg/kg/day, 1.0 mg/kg/day to 5 mg/kg/day, 2 mg/kg/day to 50 mg/kg/day, 2 mg/kg/day to 25 mg/kg/day, 2 mg/kg/day to 20 mg/kg/day, 2 mg/kg/day to 15 mg/kg/day, 2 mg/kg/day to 10 mg/kg/day, 2 mg/kg/day to 7.5 mg/kg/day, or 2 mg/kg/day to 5 mg/kg/day.

The compounds can be administered at a dosage in the range from about 0.25 milligram/kg/day to about 25 mg/kg/day. For example, the dosage may be 0.25 mg/kg/day, 0.5 mg/kg/day, 0.75 mg/kg/day, 1.0 mg/kg/day, 1.25 mg/kg/day, 1.5 mg/kg/day, 1.75 mg/kg/day, 2.0 mg/kg/day, 2.25 mg/kg/day, 2.5 mg/kg/day, 2.75 mg/kg/day, 3.0 mg/kg/day, 3.25 mg/kg/day, 3.5 mg/kg/day, 3.75 mg/kg/day, 4.0 mg/kg/day, 4.25 mg/kg/day, 4.5 mg/kg/day, 4.75 mg/kg/day, 5 mg/kg/day, 5.5 mg/kg/day, 6.0 mg/kg/day, 6.5 mg/kg/day, 7.0 mg/kg/day, 7.5 mg/kg/day, 8.0 mg/kg/day, 8.5 mg/kg/day, 9.0 mg/kg/day, 9.5 mg/kg/day, 10 mg/kg/day, 11 mg/kg/day, 12 mg/kg/day, 13 mg/kg/day, 14 mg/kg/day, 15 mg/kg/day, 16 mg/kg/day, 17 mg/kg/day, 18 mg/kg/day, 19 mg/kg/day, 20 mg/kg/day, 21 mg/kg/day, 22 mg/kg/day, 23 mg/kg/day, 24 mg/kg/day, 25 mg/kg/day, 26 mg/kg/day, 27 mg/kg/day, 28 mg/kg/day, 29 mg/kg/day, 30 mg/kg/day, 31 mg/kg/day, 32 mg/kg/day, 33 mg/kg/day, 34 mg/kg/day, 35 mg/kg/day, 36 mg/kg/day, 37 mg/kg/day, 38 mg/kg/day, 39 mg/kg/day, 40 mg/kg/day, 41 mg/kg/day, 42 mg/kg/day, 43 mg/kg/day, 44 mg/kg/day, 45 mg/kg/day, 46 mg/kg/day, 47 mg/kg/day, 48 mg/kg/day, 49 mg/kg/day, or 50 mg/kg/day.

The compound or precursor thereof can be administered in concentrations that range from 0.01 micromolar to greater than or equal to 500 micromolar. For example, the dose may be 0.01 micromolar, 0.02 micromolar, 0.05 micromolar, 0.1 micromolar, 0.15 micromolar, 0.2 micromolar, 0.5 micromolar, 0.7 micromolar, 1.0 micromolar, 3.0 micromolar, 5.0 micromolar, 7.0 micromolar, 10.0 micromolar, 15.0 micromolar, 20.0 micromolar, 25.0 micromolar, 30.0 micromolar, 35.0 micromolar, 40.0 micromolar, 45.0 micromolar, 50.0 micromolar, 60.0 micromolar, 70.0 micromolar, 80.0 micromolar, 90.0 micromolar, 100.0 micromolar, 150.0 micromolar, 200.0 micromolar, 250.0 micromolar, 300.0 micromolar, 350.0 micromolar, 400.0 micromolar, 450.0 micromolar, to greater than about 500.0 micromolar or any incremental value thereof. It is to be understood that all values and ranges between these values and ranges are meant to be encompassed.

The compound or precursor thereof can be administered at concentrations that range from 0.10 microgram/mL to 500.0 microgram/mL. For example, the concentration may be 0.10 microgram/mL, 0.50 microgram/mL, 1 microgram/mL, 2.0 microgram/mL, 5.0 microgram/mL, 10.0 microgram/mL, 20 microgram/mL, 25 microgram/mL. 30 microgram/mL, 35 microgram/mL, 40 microgram/mL, 45 microgram/mL, 50 microgram/mL, 60.0 microgram/mL, 70.0 microgram/mL, 80.0 microgram/mL, 90.0 microgram/mL, 100.0 microgram/mL, 150.0 microgram/mL, 200.0 microgram/mL, 250.0 g/mL, 250.0 micro gram/mL, 300.0 microgram/mL, 350.0 microgram/mL, 400.0 microgram/mL, 450.0 microgram/mL, to greater than about 500.0 microgram/mL or any incremental value thereof. It is to be understood that all values and ranges between these values and ranges are meant to be encompassed.

The formulations can be administered in pharmaceutically acceptable solutions, which can routinely contain pharmaceutically acceptable concentrations of salt, buffering agents, preservatives, compatible carriers, adjuvants, and optionally other therapeutic ingredients. For use in therapy, an effective amount of the compound can be administered to a subject by any mode that delivers the compound to the desired surface.

Administering a pharmaceutical composition can be accomplished by any means known to the skilled artisan. Routes of administration include, but are not limited to, intravenous, intramuscular, intraperitoneal, intravesical (urinary bladder), oral, subcutaneous, direct injection (for example, into a tumor or abscess), mucosal (e.g., topical to eye), inhalation, and topical.

For intravenous and other parenteral routes of administration, a compound can be formulated as a lyophilized preparation, as a lyophilized preparation of liposome-intercalated or -encapsulated active compound, as a lipid complex in aqueous suspension, or as a salt complex. Lyophilized formulations are generally reconstituted in suitable aqueous solution, e.g., in sterile water or saline, shortly prior to administration.

For oral administration, the compounds can be formulated readily by combining the active compound(s) with pharmaceutically acceptable carriers well-known in the art. Such carriers enable the compounds to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents can be added, such as the cross-linked polyvinyl pyrrolidone, agar, or alginic acid or a salt thereof such as sodium alginate. Optionally the oral formulations can also be formulated in saline or buffers, e.g., EDTA for neutralizing internal acid conditions, or can be administered without any carriers.

Also contemplated are oral dosage forms of the compounds. The compounds can be chemically modified so that oral delivery of the derivative is efficacious. Generally, the chemical modification contemplated is the attachment of at least one moiety to the compound itself, where said moiety permits (a) inhibition of acid hydrolysis; and (b) uptake into the blood stream from the stomach or intestine. Also desired is the increase in overall stability of the compounds and increase in circulation time in the body. Examples of such moieties include polyethylene glycol, copolymers of ethylene glycol and propylene glycol, carboxymethyl cellulose, dextran, polyvinyl alcohol, polyvinyl pyrrolidone and polyproline. Abuchowski and Davis, “Soluble Polymer-Enzyme Adducts,” In: Enzymes as Drugs, Hocenberg and Roberts, eds., Wiley-Interscience, New York, N.Y., pp. 367-383 (1981); Newmark et al., J Appl Biochem 4:185-189 (1982). Other polymers that could be used are poly-1,3-dioxolane and poly-1,3,6-tioxocane. For pharmaceutical usage, as indicated above, polyethylene glycol moieties are suitable.

The location of release of a compound may be the stomach, the small intestine (the duodenum, the jejunum, or the ileum), or the large intestine. One skilled in the art has available formulations, which will not dissolve in the stomach, yet will release the material in the duodenum or elsewhere in the intestine. The release can avoid the deleterious effects of the stomach environment, either by protection of the compound or by release of the compound beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and shellac. These coatings may be used as mixed films.

A coating or mixture of coatings can also be used on tablets, which are not intended for protection against the stomach. This can include sugar coatings, or coatings which make the tablet easier to swallow. Capsules can consist of a hard shell (such as gelatin) for delivery of dry therapeutic (e.g., powder); for liquid forms, a soft gelatin shell can be used. The shell material of cachets could be thick starch or other edible paper. For pills, lozenges, molded tablets or tablet triturates, moist massing techniques can be used.

The therapeutic agent can be included in the formulation as fine multi-particulates in the form of granules or pellets of particle size about 1 mm. The formulation of the material for capsule administration could also be as a powder, lightly compressed plugs or even as tablets. The therapeutic agent could be prepared by compression.

Colorants and flavoring agents may all be included. For example, the compound may be formulated (such as by liposome or microsphere encapsulation) and then further contained within an edible product, such as a refrigerated beverage containing colorants and flavoring agents.

One may dilute or increase the volume of the therapeutic agent with an inert material. These diluents can include carbohydrates, especially mannitol, a-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants can be included in the formulation of the therapeutic agent into a solid dosage form. Materials used as disintegrates include, but are not limited to, starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrant is the insoluble cationic exchange resin. Powdered gums can be used as disintegrants and as binders and these can include powdered gums such as agar, Karaya or tragacanth. Alginic acid and its sodium salt are also useful as disintegrants.

Binders can be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) can both be used in alcoholic solutions to granulate the therapeutic agent.

An anti-frictional agent can be included in the formulation of the therapeutic to prevent sticking during the formulation process. Lubricants can be used as a layer between the therapeutic agent and the die wall, and these can include, but are not limited to, stearic acid, including its magnesium and calcium salts, polytetrafluoroethylene (PTFE), liquid paraffin, vegetable oils and waxes. Soluble lubricants can also be used, such as sodium lauryl sulfate, magnesium lauryl sulfate, polyethylene glycol of various molecular weights, Carbowax 4000 and 6000.

Glidants, which can improve the flow properties of the drug during formulation and aid rearrangement during compression, can be added. The glidants can include starch, talc, pyrogenic silica and hydrated silicoaluminate.

To aid dissolution of the therapeutic agent into the aqueous environment a surfactant can be added as a wetting agent. Surfactants can include anionic detergents, such as sodium lauryl sulfate, dioctyl sodium sulfosuccinate and dioctyl sodium sulfonate.

Cationic detergents which can be used include benzalkonium chloride and benzethonium chloride. Potential non-ionic detergents that can be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compound or derivative thereof either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. Microspheres formulated for oral administration can also be used. Such microspheres have been well defined in the art. All formulations for oral administration should be in dosages suitable for such administration.

For buccal administration, the compositions can take the form of tablets or lozenges formulated in conventional manner.

For topical administration, the compound can be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

For administration by inhalation, compounds can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

Also contemplated is pulmonary delivery of the compounds (or salts thereof). The compound is delivered to the lungs of a mammal while inhaling and traverses across the lung epithelial lining to the blood stream. Other reports of inhaled molecules include Adjei et al., Pharm Res 7:565-569 (1990); Adjei et al., Int J Pharmaceutics 63:135-144 (1990) (leuprolide acetate); Braquet et al., J Cardiovasc Pharmacol 13 (suppl. 5): 143-146 (1989) (endothelin-1); Hubbard et al., Annal Int Med 3:206-212 (1989) (al-antitrypsin); Smith et al., 1989, J Clin Invest 84:1145-1146 (a-1-proteinase); Oswein et al., 1990, “Aerosolization of Proteins,” Proceedings of Symposium on Respiratory Drug Delivery II, Keystone, Colo., March, (recombinant human growth hormone); Debs et al., 1988, J Immunol 140:3482-3488 (interferon-gamma and tumor necrosis factor alpha) and Platz et al., U.S. Pat. No. 5,284,656 (granulocyte colony stimulating factor; incorporated by reference). A method and composition for pulmonary delivery of drugs for systemic effect is described in U.S. Pat. No. 5,451,569 (specifically incorporated by reference for its disclosure regarding same), issued Sep. 19, 1995, to Wong et al.

Contemplated for use are a wide range of mechanical devices designed for pulmonary delivery of therapeutic products, including but not limited to nebulizers, metered dose inhalers, and powder inhalers, all of which are familiar to those skilled in the art.

Nasal delivery of a pharmaceutical composition is also contemplated. Nasal delivery allows the passage of a pharmaceutical composition to the blood stream directly after administering the therapeutic product to the nose, without the necessity for deposition of the product in the lung. Formulations for nasal delivery include those with dextran or cyclodextran.

The compounds, when it is desirable to deliver them systemically, can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

Pharmaceutical formulations for parenteral administration include aqueous solutions of the active compounds in water-soluble form. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethylcellulose, sorbitol, or dextran. Optionally, the suspension can also contain suitable stabilizers or agents which increase the solubility of the compounds to allow for the preparation of highly concentrated solutions.

Alternatively, the active compounds can be in powder form for constitution with a suitable vehicle, e.g., sterile pyrogen-free water, before use.

The compounds can also be formulated in rectal or vaginal compositions such as suppositories or retention enemas, e.g., containing conventional suppository bases such as cocoa butter or other glycerides.

In addition to the formulations described above, a compound can also be formulated as a depot preparation. Such long-acting formulations can be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also can comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer R, Science 249:1527-1533 (1990).

The compound and optionally one or more other therapeutic agents can be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Pharmaceutical compositions contain an effective amount of a compound as described herein and optionally one or more other therapeutic agents included in a pharmaceutically acceptable carrier. The term “pharmaceutically acceptable carrier” means one or more compatible solid or liquid fillers, diluents or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also can be commingled with the compounds, and with each other, in a manner such that there is no interaction which would substantially impair the desired pharmaceutical efficiency.

The therapeutic agent(s), including specifically, but not limited to, a compound, may be provided in particles. “Particles” as used herein means nanoparticles or microparticles (or in some instances larger particles) which can consist in whole or in part of the compound or the other therapeutic agent(s) as described herein. The particles can contain the therapeutic agent(s) in a core surrounded by a coating, including, but not limited to, an enteric coating. The therapeutic agent(s) also can be dispersed throughout the particles. The therapeutic agent(s) also can be adsorbed into the particles. The particles can be of any order release kinetics, including zero-order release, first-order release, second-order release, delayed release, sustained release, immediate release, and any combination thereof, etc. The particle can include, in addition to the therapeutic agent(s), any of those materials routinely used in the art of pharmacy and medicine, including, but not limited to, erodible, nonerodible, biodegradable, or nonbiodegradable material or combinations thereof. The particles can be microcapsules which contain the compound in a solution or in a semi-solid state. The particles can be of virtually any shape.

Both non-biodegradable and biodegradable polymeric materials can be used in the manufacture of particles for delivering the therapeutic agent(s). Such polymers can be natural or synthetic polymers. The polymer is selected based on the period of time over which release is desired. Bioadhesive polymers of particular interest include bioerodible hydrogels described in Sawhney et al., Macromolecules 26:581-587 (1993), the teachings of which are specifically incorporated by reference herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) can be contained in controlled-release systems. The term “controlled release” is intended to refer to any drug-containing formulation in which the manner and profile of drug release from the formulation are controlled. This refers to immediate as well as non-immediate release formulations, with non-immediate release formulations including, but not limited to, sustained release and delayed release formulations. The term “sustained release” (also referred to as “extended release”) is used in its conventional sense to refer to a drug formulation that provides for gradual release of a drug over an extended period of time, and that can result in substantially constant blood levels of a drug over an extended time period. The term “delayed release” is used in its conventional sense to refer to a drug formulation in which there is a time delay between administration of the formulation and the release of the drug therefrom. “Delayed release” may or may not involve gradual release of drug over an extended period of time, and thus may or may not be “sustained release.”

Use of a long-term sustained release implant can be particularly suitable for treatment of chronic conditions. “Long-term” release, as used herein, means that the implant is constructed and arranged to deliver therapeutic levels of the active ingredient for at least 7 days, and up to 30-60 days. Long-term sustained release implants are well-known to those of ordinary skill in the art and include some of the release systems described above.

It will be understood by one of ordinary skill in the relevant arts that other suitable modifications and adaptations to the compositions and methods described herein are readily apparent from the description contained herein in view of information known to the ordinarily skilled artisan, and may be made without departing from the scope of the disclosure or any embodiment thereof. Having now described the present disclosure in detail, the same will be more clearly understood by reference to the following examples, which are included herewith for purposes of illustration only and are not intended to be limiting of the disclosure.

### EXAMPLES

### Chemical Examples

### Example 1

### Synthesis of trans-2-((3S,5S)-5-((S)-2-cyano-4,4-difluoropyrrolidine-1-carbonyl)-2-oxopyrrolidin-3-yl)acetic acid

trans-2-((3S,5S)-5-((S)-2-cyano-4,4-difluoropyrrolidine-1-carbonyl)-2-oxopyrrolidin-3-yl)acetic acid was synthesized according to Scheme 1.

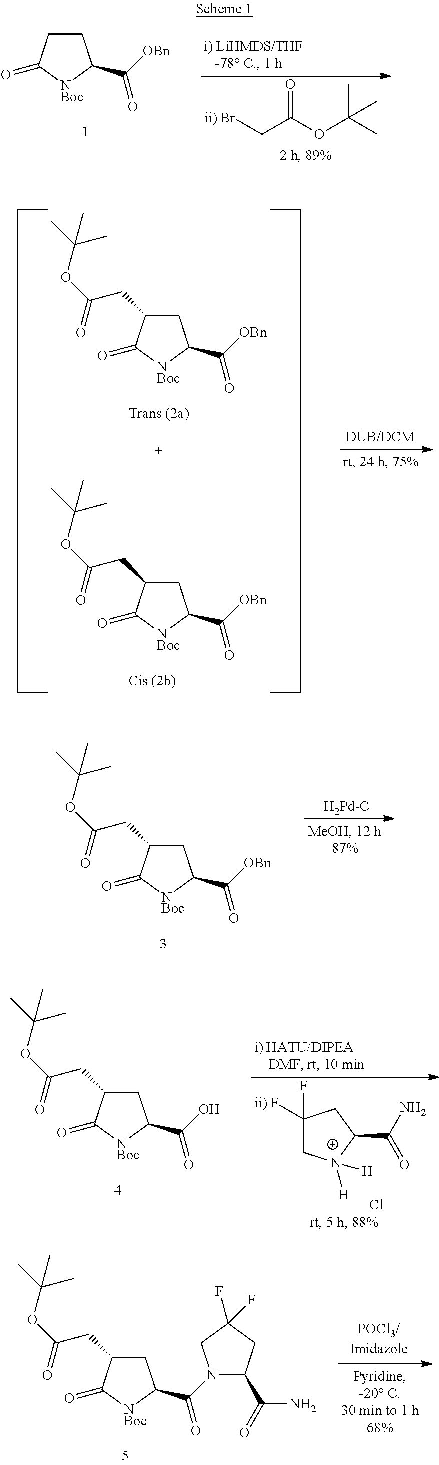

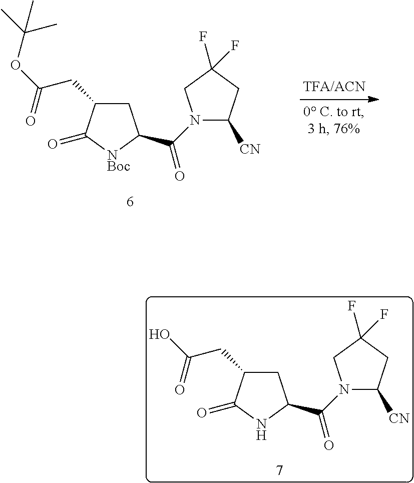 [SMILES: CC(C)(C)OC(=O)C[C@@H]1C[C@@H](C(=O)N2CC(F)(F)C[C@H]2C#N)N(C(=O)OC(C)(C)C)C1=O ; N#C[C@@H]1CC(F)(F)CN1C(=O)[C@@H]1C[C@@H](CC(=O)O)C(=O)N1]

### Step-I:

Benzyl (2S)—N-tert-butoxylcarbonylpyroglutamate (Compound 1, 5 grams (g), 15.67 millimoles (mmol)) was dissolved in tetrahydrofuran (THF, 75 milliliters (mL)) and cooled to −78° C., with stirring under nitrogen. Lithium bis(trimethylsilyl)amide (LiHMDS) (1.0 M in THF, 34.5 mL, 34.5 mmol) was added dropwise over 5 minutes (min) and stirring was continued for 1 hour (h). Tert-Butyl bromoacetate (4.36 mL, 31.34 mmol) was added dropwise over 5 min, and stirring was continued at −78° C. for an additional 2 h. The reaction mixture was quenched with saturated aqueous ammonium chloride (100 mL) and extracted into ethyl acetate (2×100 mL). The organic layer was washed with water and saturated aqueous sodium chloride and then dried over anhydrous magnesium sulphate (MgSO₄). The solvent was removed in vacuo to give a brown oil, which was purified by combi flash on silica gel chromatography (eluted with hexane/EtOAc) to yield a mixture of compound 2a (trans-2-benzyl 1-(tert-butyl) (2S,4S)-4-(2-(tert-butoxy)-2-oxoethyl)-5-oxopyrrolidine-1,2-dicarboxylate) and compound 2b (cis-2-benzyl 1-(tert-butyl) (2S,4S)-4-(2-(tert-butoxy)-2-oxoethyl)-5-oxopyrrolidine-1,2-dicarboxylate) (6.2 gm, 89%) as gummy liquid.

### Step-II:

A mixture of compounds 2a/2b (6 g, 13.85 mmol) was dissolved in CH₂Cl₂ (90 mL) and cooled to 0° C. DBU (6.2 mL, 41.55 mmol) was added dropwise and the mixture stirred at 0° C. for 30 min and then at room temperature for 24 h. The reaction mixture was diluted with CH₂Cl₂ (20 mL) and washed with water (100 mL). The organic layer was dried over MgSO₄, filtered, and concentrated in vacuo to give the crude product, which was purified by column chromatography as described in step I to yield trans-2-benzyl 1-(tert-butyl) (2S,4S)-4-(2-(tert-butoxy)-2-oxoethyl)-5-oxopyrrolidine-1,2-dicarboxylate (compound 3, 4.5 g, 75%) as pale yellow liquid.

### Step-III:

To a solution of compound 3 (4 g, 9.23 mmol) in MeOH (40 mL) was added 5% Pd/C (400 mg) under an atmosphere of nitrogen. The mixture was stirred vigorously under an atmosphere of hydrogen for 12 h at room temperature. The mixture was filtered through a Celite pad and concentrated in vacuo to give trans-(2S,4S)-4-(2-(tert-butoxy)-2-oxoethyl)-1-(tert-butoxycarbonyl)-5-oxopyrrolidine-2-carboxylic acid (compound 4, 2.7 g, 87%) as white solid.

### Step-IV:

To a stirred solution of compound 4 (2.5 g, 7.28 mmol) in dry DMF (20 mL) was added HATU (3.3 g, 8.7 mmol), DIPEA (3.6 mL, 21.84 mmol) and stirring continued there for 10 min for activation of acid functionality. (S)-4,4-Difluoropyrrolidine-2-carboxamidehydrochloride (1.3 g, 8.736 mmol) followed by DIPEA (1.46 mL, 8.73 mmol) was added to above reaction mixture and stirring continued at room temperature, under nitrogen atmosphere for 5 h. The reaction mixture was diluted with water (30 mL), brine (30 mL), and extracted into ethyl acetate (2×50 mL). The combined organic extracts were dried over anhydrous sodium sulphate, filtered and the filtrate was evaporated under reduced pressure, and the obtained crude residue was purified by combi flash using DCM/MeOH as mobile phase to provide trans-tert-butyl (3S,5S)-3-(2-(tert-butoxy)-2-oxoethyl)-5-((S)-2-carbamoyl-4,4-difluoropyrrolidine-1-carbonyl)-2-oxopyrrolidine-1-carboxylate (5, 2.4 g 88%) as gummy solid.

### Step-V:

To a mixture of compound 5 (2 g, 4.21 mmol) and imidazole (333 milligrams (mg), 4.73 mmol) in pyridine (13 mL) cooled to −20° C. was added phosphoryl chloride (POCl₃) (1.02 mL, 10.94 mmol) under nitrogen. After being stirred for 30 min to 1 h at −20° C., the mixture was evaporated to dryness in vacuo. The resulting brown solid was dissolved in CH₂Cl₂ (40 mL) and washed with 1.0 N aqueous citric acid (40 mL). The organic phase was dried over magnesium sulphate, filtered, and concentrated under reduced pressure to yield the crude material as a viscous oil. The crude material was purified by combi flash (eluted with hexane/EtOAc) to give trans-tert-butyl (3S,5S)-3-(2-(tert-butoxy)-2-oxoethyl)-5-((S)-2-cyano-4,4-difluoropyrrolidine-1-carbonyl)-2-oxopyrrolidine-1-carboxylate (compound 6, 1.3 g, 68%) as a white solid.

### Step-VI:

To a solution of compound 6 (1.0 g, 2.1 mmol) in acetonitrile (CH₃CN) (5 mL) at 0° C. was added TFA (5 mL) dropwise over 5 min. The reaction mixture was stirred for 1 h at room temperature. The reaction mixture was concentrated under vacuum and crystallized from ethyl acetate (EA)/ether to yield trans-2-((3S,5S)-5-((S)-2-cyano-4,4-difluoropyrrolidine-1-carbonyl)-2-oxopyrrolidin-3-yl)acetic acid (compound 7, 500 mg, 76%) as white powder.

### Example 2

### Synthesis of tert-butyl 4-(azidomethyl)isoindoline-2-carboxylate and tert-butyl 4-(aminomethyl)isoindoline-2-carboxylate

tert-butyl 4-(azidomethyl)isoindoline-2-carboxylate and tert-butyl 4-(aminomethyl)isoindoline-2-carboxylate were synthesized according to Scheme 2.

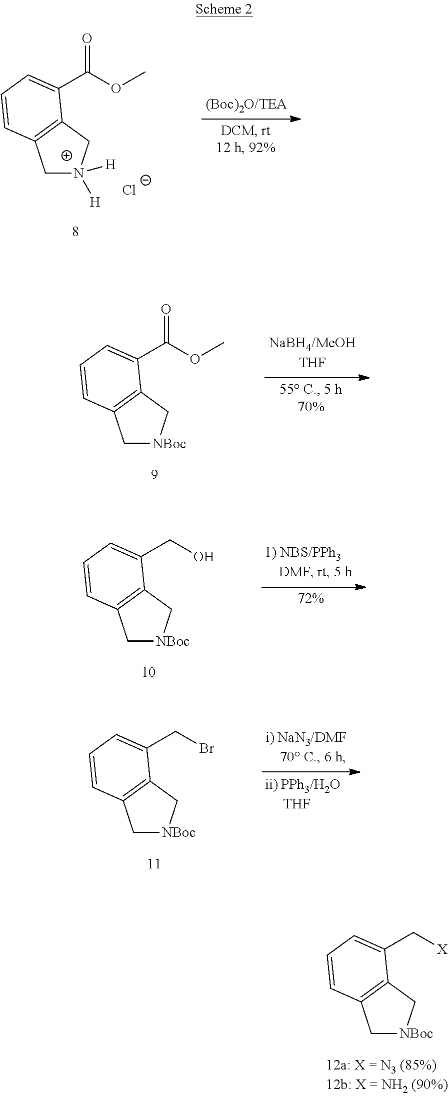

### Step-I

To a stirred solution of isoindoline methylester hydrochloride (compound 8, 1.00 g, 5.64 mmol) in DCM (20 mL) at room temperature was added Boc₂O (4.9 mL, 22.59 mmol) in one portion followed by triethylamine dropwise (2.9 mL, 22.59 mmol). Stirring was continued for 12 h, and the reaction mixture was diluted with water (30 mL) and extracted into DCM (2×25 mL). The organic layer was dried over anhydrous MgSO₄ and filtered, and the filtrate was evaporated under reduced pressure. The obtained crude residue was purified by combi flash using hexanes and ethyl acetate as mobile phase to provide 2-(tert-butyl) 4-methyl isoindoline-2,4-dicarboxylate (compound 9, 1.2 g, 92%) as white colourless gummy liquid. LC-MS (m/z); [M+H] calcd for C₁₅H₂₀NO₄ found: 278.13 g/mol.

### Step-II:

To a stirred solution of 2-(tert-butyl) 4-methyl isoindoline-2,4-dicarboxylate (compound 9, 1.0 g, 3.61 mmol) in THF (10.0 mL) at room temperature was added sodium borohydride (1.37 g, 36.101 mmol) in an atmosphere of nitrogen. To the stirring mixture, methanol (MeOH, 10 mL) was added dropwise over 5 minutes. The reaction was warmed to 55° C. and stirred for 5 h. The reaction mixture was cooled to 0° C. and slowly quenched with saturated aqueous ammonium chloride and extracted into EtOAc (60 mL). The organic phase was collected, dried over sodium sulfate and the solvent distilled to provide crude residue which was purified by combi flash to yield tert-butyl 4-(hydroxymethyl)isoindoline-2-carboxylate (compound 10, 700 mg, 70%) as white gummy solid. LC-MS (m/z): [M+H]: calcd for: C₁₄H₂₀NO₃ found: 250.14 g/mol.

### Step-III:

Tert-butyl 4-(hydroxymethyl)isoindoline-2-carboxylate (compound 10, 500 mg, 2.00 mmol) was dissolved in DMF (10 mL) followed by PPh₃ (790 mg, 3.01 mmol) and freshly recrystallized NBS (532 mg, 3.01 mmol) was added. The reaction mixture was stirred at room temperature under nitrogen atmosphere for 4 to 5 h. The reaction mixture was diluted with water (40 mL) and extracted into ethyl acetate (2×25 mL), and the organic layer was washed with water and brine, dried over anhydrous sodium sulphate, and filtered. The filtrate was evaporated under reduced pressure and purified by combi flask to provide tert-butyl 4-(bromomethyl)isoindoline-2-carboxylate (compound 11, 450 mg, 72%) as white solid. LC-MS (m/z): [M+H] calcd for: C₁₄H₁₉BrNO₂ found: 312.05 g/mol.

### Step-IV:

To a stirred solution of tert-butyl 4-(bromomethyl)isoindoline-2-carboxylate (compound 11, 400 mg, 1.286 mmol) in DMF was added NaN₃ (420 mg, 6.430 mmol), then stirring continued at 65° C. for 6 h. The reaction mixture was diluted with water and extracted into ethyl acetate, the organic layer was washed with water and brine, dried over anhydrous sodium sulphate, and filtered. The filtrate was evaporated under reduced pressure and purified by combi flask to give tert-butyl 4-(azidomethyl)isoindoline-2-carboxylate (compound 12a, 300 mg, 85%) as colorless gummy liquid. LC-MS (m/z): [M+H] calcd for C₁₄H₁₉N₄O₂ found: 274.14 g/mol.

### Step-V:

To a stirred solution of tert-butyl 4-(azidomethyl)isoindoline-2-carboxylate (compound 12a, 1.0 equivalents (eq)) THF was added followed by PPh₃ (1.5 eq) and water (3.0 eq) and stirring continued at room temperature for 12 h. The reaction mixture was evaporated under reduced pressure and the obtained crude residue was purified by using combi flask using methanol and dichloromethane as mobile phase to provide tert-butyl 4-(aminomethyl)isoindoline-2-carboxylate (compound 12b, 85%) as white solid. LC-MS (m/z): [M+H] calcd for C₁₄H₂₁N₂O₂ found: 249.15 g/mol.

### Example 3

5-((2-(4-(((2-(2-((3S,5S)-5-((S)-2-cyano-4,4-difluoropyrrolidine-1-carbonyl)-2-oxopyrrolidin-3-yl)acetyl)isoindolin-4-yl)methyl)amino)-4-oxobutanamido)ethyl)carbamoyl)-2-(6-(dimethylamino)-3-(dimethyliminio)-9,9a-dihydro-3H-xanthen-9-yl)benzoate was synthesized according to Scheme 3.

### Synthesis of 5-((2-(4-(((2-(2-((3S,5S)-5-((S)-2-cyano-4,4-difluoropyrrolidine-1-carbonyl)-2-oxopyrrolidin-3-yl)acetyl)isoindolin-4-yl)methyl)amino)-4-oxobutanamido)ethyl)carbamoyl)-2-(6-(dimethylamino)-3-(dimethyliminio)-9,9a-dihydro-3H-xanthen-9-yl)benzoate

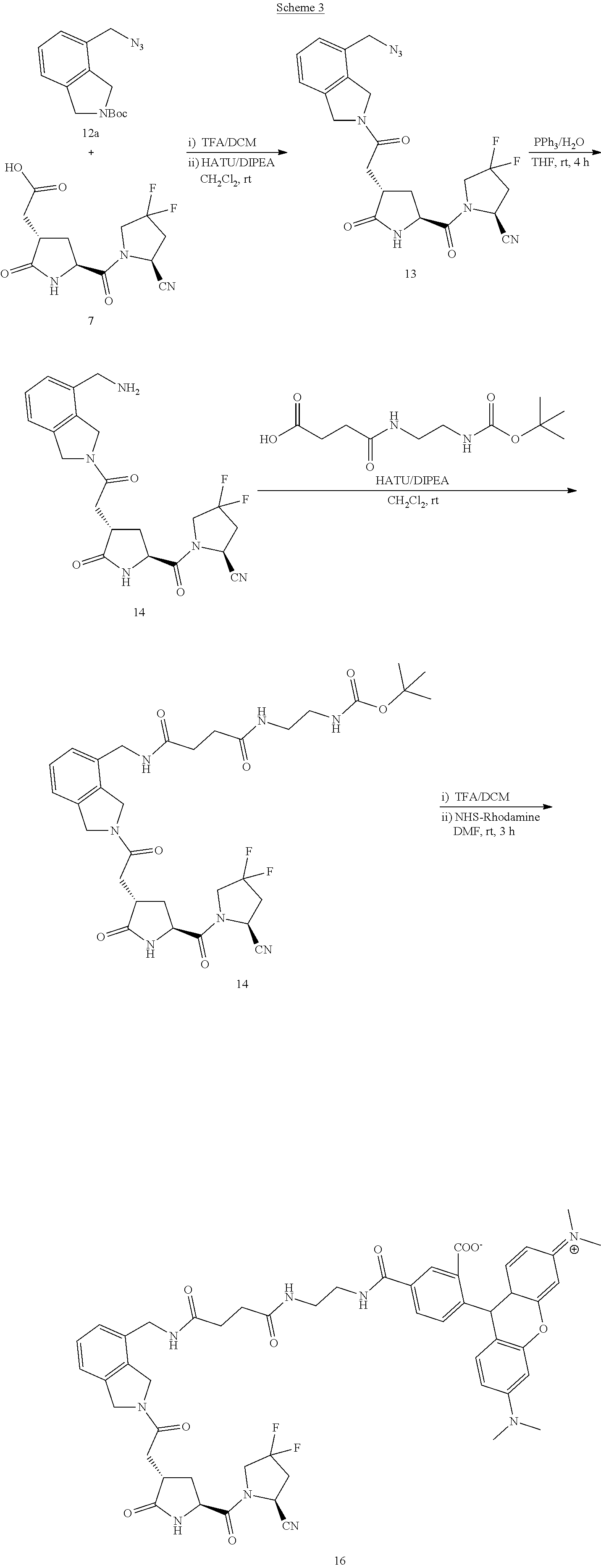

### Step-I

To a stirred solution of tert-butyl 4-(azidomethyl)isoindoline-2-carboxylate (compound 12a) in DCM was added trifluoroacetic acid (TFA) and stirring was continued for 1 h. In another flask trans-2-((3S,5S)-5-((S)-2-cyano-4,4-difluoropyrrolidine-1-carbonyl)-2-oxopyrrolidin-3-yl)acetic acid (compound 7) was dissolved in DMF, followed by addition of HATU (1.3 eq) and DIPEA (3.0 eq) for pre-activation of acid functionality. The resulting amine was added to the mixture comprising the pre-activated acid and the mixture was stirred at room temperature for 3 h. The reaction mixture was diluted with water, and extracted into ethyl acetate. The organic layer was evaporated and the obtained crude residue was purified by combiflash using methanol and DCM as mobile phase to provide (S)-1-((2S,4S)-4-(2-(4-(azidomethyl)isoindolin-2-yl)-2-oxoethyl)-5-oxopyrrolidine-2-carbonyl)-4,4-difluoropyrrolidine-2-carbonitrile (compound 13). LC-MS (m/z): calcd for C₂₁H₂₂F2N₇O₃ found: 458.17.

### Step-II:

Using the same procedure as provided in Example 2, Step V, (S)-1-((2S,4S)-4-(2-(4-(azidomethyl)isoindolin-2-yl)-2-oxoethyl)-5-oxopyrrolidine-2-carbonyl)-4,4-difluoropyrrolidine-2-carbonitrile (compound 13) was reacted with triphenylphosphine (PPh₃), water, and tetrahydrofuran to produce (S)-1-((2S,4S)-4-(2-(4-(aminomethyl)isoindolin-2-yl)-2-oxoethyl)-5-oxopyrrolidine-2-carbonyl)-4,4-difluoropyrrolidine-2-carbonitrile (compound 14), which was carried to the next step without purification. (S)-1-((2S,4S)-4-(2-(4-(aminomethyl)isoindolin-2-yl)-2-oxoethyl)-5-oxopyrrolidine-2-carbonyl)-4,4-difluoropyrrolidine-2-carbonitrile (compound 14) was reacted with 4-((2-((tert-butoxycarbonyl)amino)ethyl)amino)-4-oxobutanoic acid, HATU (1.3 eq), DIPEA (3.0 eq), and DCM to provide tert-butyl (2-(4-(((2-(2-((3S,5S)-5-((S)-2-cyano-4,4-difluoropyrrolidine-1-carbonyl)-2-oxopyrrolidin-3-yl)acetyl)isoindolin-4-yl)methyl)amino)-4-oxobutanamido)ethyl)carbamate (compound 15).

### Step-III

To a stirred solution of tert-butyl (2-(4-(((2-(2-((3S,5S)-5-((S)-2-cyano-4,4-difluoropyrrolidine-1-carbonyl)-2-oxopyrrolidin-3-yl)acetyl)isoindolin-4-yl)methyl)amino)-4-oxobutanamido)ethyl)carbamate (compound 15) in DCM was added TFA and stirring was continued for 10 min. The reaction mixture was evaporated under reduced pressure and the obtained crude residue was treated with NHS rhodamine (1.0 eq) and DIPEA (2.0 eq) in DMF for 1 h. The reaction mixture was diluted with water and purified by UHPLC (A=20 mM ammonium acetate buffer (pH=7), B=acetonitrile, solvent gradients 5% B to 95% in 60 min to provide 5-((2-(4-(((2-(2-((3S,5S)-5-((S)-2-cyano-4,4-difluoropyrrolidine-1-carbonyl)-2-oxopyrrolidin-3-yl)acetyl)isoindolin-4-yl)methyl)amino)-4-oxobutanamido)ethyl)carbamoyl)-2-(6-(dimethylamino)-3-(dimethyliminio)-9,9a-dihydro-3H-xanthen-9-yl)benzoate (compound 16). LC-MS (m/z): [M+H] calcd for C₅₂H₅₆F₂N₉O₉ found: 988.41 g/mol.

### Example 4

N-(9-(2-carboxy-4-((1-(4-(1-((2-(2-((3S,5S)-5-((S)-2-cyano-4,4-difluoropyrrolidine-1-carbonyl)-2-oxopyrrolidin-3-yl)acetyl)isoindolin-4-yl)methyl)-1H-1,2,3-triazol-4-yl)phenyl)-1,6-dioxo-9,12,15,18-tetraoxa-2,5-diazaicosan-20-yl)carbamoyl)phenyl)-6-(dimethylamino)-9,9a-dihydro-3H-xanthen-3-ylidene)-N-methylmethanaminium and 5-((1-(4-(1-((2-(2-((3S,5S)-5-((S)-2-cyano-4,4-difluoropyrrolidine-1-carbonyl)-2-oxopyrrolidin-3-yl)acetyl)isoindolin-4-yl)methyl)-1H-1,2,3-triazol-4-yl)phenyl)-1,6-dioxo-9,12,15,18-tetraoxa-2,5-diazaicosan-20-yl)carbamoyl)-2-(6-hydroxy-3-oxo-9,9a-dihydro-3H-xanthen-9-yl)benzoic acid were synthesized according to Scheme 4a and 4b.

### Synthesis of N-(9-(2-carboxy-4-((1-(4-(1-((2-(2-((3S,5S)-5-((S)-2-cyano-4,4-difluoropyrrolidine-1-carbonyl)-2-oxopyrrolidin-3-yl)acetyl)isoindolin-4-yl)methyl)-1H-1,2,3-triazol-4-yl)phenyl)-1,6-dioxo-9,12,15,18-tetraoxa-2,5-diazaicosan-20-yl)carbamoyl)phenyl)-6-(dimethylamino)-9,9a-dihydro-3H-xanthen-3-ylidene)-N-methylmethanaminium and 5-((1-(4-(1-((2-(2-((3S,5S)-5-((S)-2-cyano-4,4-difluoropyrrolidine-1-carbonyl)-2-oxopyrrolidin-3-yl)acetyl)isoindolin-4-yl)methyl)-1H-1,2,3-triazol-4-yl)phenyl)-1,6-dioxo-9,12,15,18-tetraoxa-2,5-diazaicosan-20-yl)carbamoyl)-2-(6-hydroxy-3-oxo-9,9a-dihydro-3H-xanthen-9-yl)benzoic acid

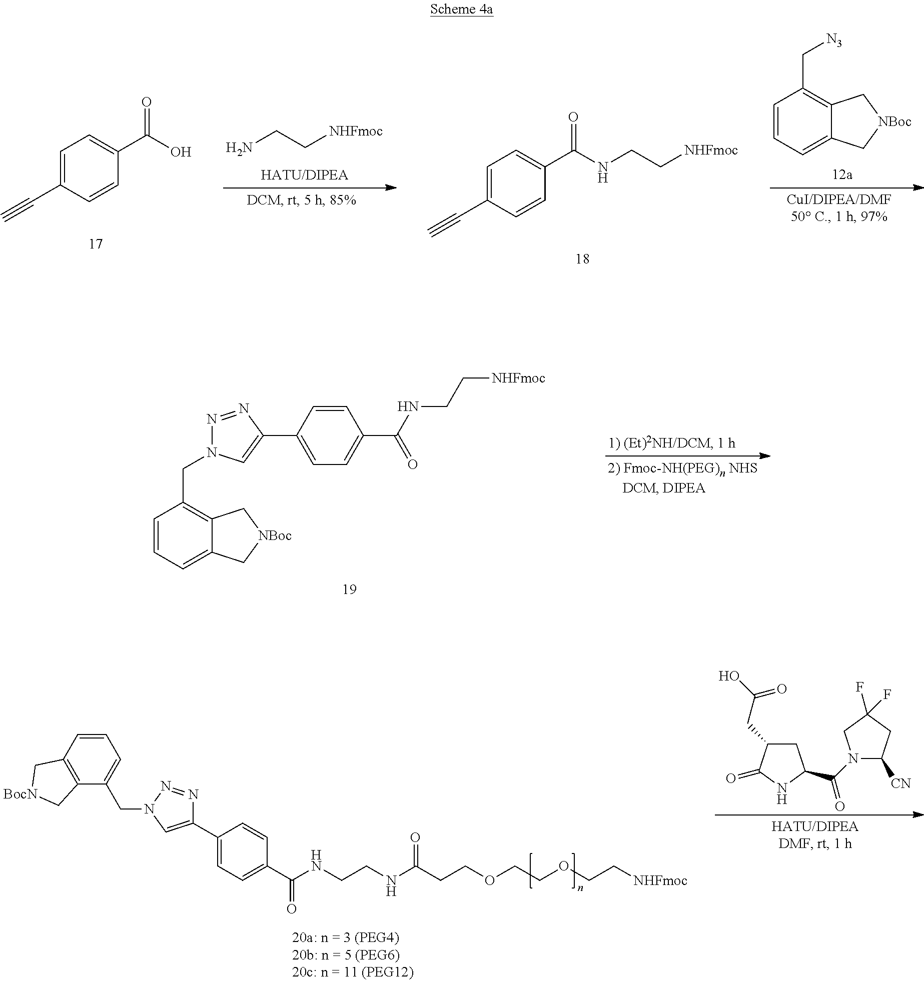

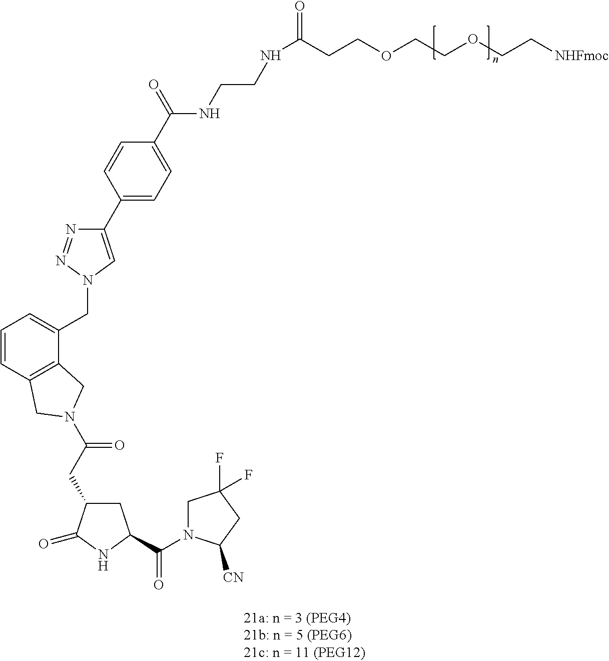

### Step-I

To a stirred solution of 4-ethynylbenzoic acid (compound 17, 500 mg, 3.424 mmol) in dry DCM (10 mL) at room temperature under nitrogen atmosphere was added HATU (1.4 gm, 3.76 mmol) followed by DIPEA (1.7 mL, 10.27 m mol) and stirring was continued for 10 min for activation of the acid. N-Fmoc-ethylenediamine (1.0 g, 3.76 mmol) was added to the reaction mixture and stirring was continued for an additional 3 h. The reaction mixture was diluted with DCM (20 mL) and the obtained precipitate was filtered through a Buchner funnel. The resulting white solid was washed with DCM (2×20 mL) again and dried under vacuum for 1 h to provide (9H-fluoren-9-yl)methyl (2-(4-ethynylbenzamido)ethyl)carbamate (compound 18, 1.2 g, 85%). LRMS-LC/MS (m/z): [M+H] calcd for C₂₆H₂₃N₂O₃ found 411.16.

### Step-II:

To a mixture of tert-butyl 4-(azidomethyl)isoindoline-2-carboxylate (compound 12a, 200 mg, 0.729 mmol) and (9H-fluoren-9-yl)methyl (2-(4-ethynylbenzamido)ethyl)carbamate (compound 18, 360 mg, 0.874 mmol) in dry DMF (5.0 mL) was added copper iodide (CuI, 70 mg, 0.364 mmol) followed by DIPEA (0.3 mL, 1.458 mmol). The reaction mixture was stirred at 50° C. under nitrogen atmosphere for 5 h, and the mixture was cooled to room temperature, diluted with water (20 mL), and vigorously stirred for 15 min. The solid residue formed in the reaction mixture was filtered and washed with water 2×20 mL, and dried under vacuum for 1 h to provide tert-butyl 4-((4-(4-((2-((((9H-fluoren-9-yl)methoxy)carbonyl)amino)ethyl)carbamoyl)phenyl)-1H-1,2,3-triazol-1-yl)methyl)isoindoline-2-carboxylate (compound 19, 480 mg, 96%) as brown solid, which was taken to further steps without purification. LRMS-LC/MS (m/z): [M+H] calcd for C₄₀H₄₁N₆O₅ found 684.31.

### Step-III:

To a stirred solution of compound tert-butyl 4-((4-(4-((2-((((9H-fluoren-9-yl)methoxy)carbonyl)amino)ethyl)carbamoyl)phenyl)-1H-1,2,3-triazol-1-yl)methyl)isoindoline-2-carboxylate (compound 19, 400 mg, 0.584 mmol) in DCM and MeOH (1:0.5 mL) was added (Et)₂NH (1 mL) and stirring was continued for 2 h. The reaction mixture was evaporated under reduced pressure and the obtained crude residue was treated with diethyl ether (3×10 mL). Scratching of the residue was used to produce a solid. The diethyl ether was decanted, and the obtained solid was dried under vacuum for 1 h before proceeding to the next step. The amine compound was dissolved in DCM (1 mL for 1 mmol) followed by Fmoc-NH(PEG)ₙ NHS esters (1.2 equiv, n=3, 5, and 11) followed by DIPEA (2.0 equiv) and the reaction mixture was stirred at room temperature under nitrogen atmosphere for 1 h. The reaction mixture was evaporated under reduced pressure and the obtained crude residue was purified by combi flash using DCM and MeOH as mobile phase to provide the compounds 20a-c in 70-80% yield.

i) LCMS for 20a: LC/MS (m/z): [M+H] calcd for C₅₁H₆₂N₇O₁₀ found: 931.45.

ii) LCMS for 20b: LC/MS (m/z): [M+H] calcd for C₅₅H₆₉N₇O₁₂ found: 1020.50.

iii) LCMS for 20c: LC/MS (m/z): [M+H] Calcd for C₆₇H₉₃N₇O₁₈ found: 1284.66.

### Step-IV:

To a stirred solution of compounds 20a, 20b, or 20c (1.0 eq) in DCM (1.0 mL) at room temperature was added TFA (10 eq) and stirring was continued for 30 min. The reaction mixture was evaporated and dried under vacuum. In a separate round bottom flask, trans-2-((3S,5S)-5-((S)-2-cyano-4,4-difluoropyrrolidine-1-carbonyl)-2-oxopyrrolidin-3-yl)acetic acid (compound 7, 1.2 eq) was dissolved in DMF (0.5 mL) followed by HATU (1.3 eq) and DIPEA (5.0 eq) and the reaction mixture was stirred under nitrogen atmosphere at room temperature for 10 min for activation of the acid functionality in compound 7. The amines obtained from Fmoc deprotection of compounds 20a-c were dissolved in DCM (1 mL) added to the above reaction mixture and stirring was continued for an additional 2 h. The reaction mixture was diluted with water (15 mL) and extracted into ethyl acetate (2×15 mL). The organic extracts were dried over anhydrous sodium sulphate and concentrated. The obtained crude residue was purified by combi flash using DCM and MeOH as mobile phase to give the desired compounds (9H-fluoren-9-yl)methyl (1-(4-(1-((2-(2-((3S,5S)-5-((S)-2-cyano-4,4-difluoropyrrolidine-1-carbonyl)-2-oxopyrrolidin-3-yl)acetyl)isoindolin-4-yl)methyl)-1H-1,2,3-triazol-4-yl)phenyl)-1,6-dioxo-9,12,15,18-tetraoxa-2,5-diazaicosan-20-yl)carbamate, (9H-fluoren-9-yl)methyl (1-(4-(1-((2-(2-((3S,5S)-5-((S)-2-cyano-4,4-difluoropyrrolidine-1-carbonyl)-2-oxopyrrolidin-3-yl)acetyl)isoindolin-4-yl)methyl)-1H-1,2,3-triazol-4-yl)phenyl)-1,6-dioxo-9,12,15,18,21,24-hexaoxa-2,5-diazahexacosan-26-yl)carbamate, and (9H-fluoren-9-yl)methyl (1-(4-(1-((2-(2-((3S,5S)-5-((S)-2-cyano-4,4-difluoropyrrolidine-1-carbonyl)-2-oxopyrrolidin-3-yl)acetyl)isoindolin-4-yl)methyl)-1H-1,2,3-triazol-4-yl)phenyl)-1,6-dioxo-9,12,15,18,21,24,27,30,33,36,39,42-dodecaoxa-2,5-diazatetratetracontan-44-yl)carbamate in 40-60% yield.

i) LCMS for compound 21a: LC/MS (m/z): [M+H] calcd for: C₅₈H₆₅F₂N₁₀O₁₁ found: 1115.47.

ii) LCMS for compound 21b: LC/MS (m/z): [M+H] calcd for: C₆₂H₇₂F₂N₁₀O₁₃ found: 1203.52.

iii) LCMS for compound 21c: LC/MS (m/z): [M+H] calcd for: C₇₄H₉₇F₂N₁₀O₁₉ found: 1467.68.

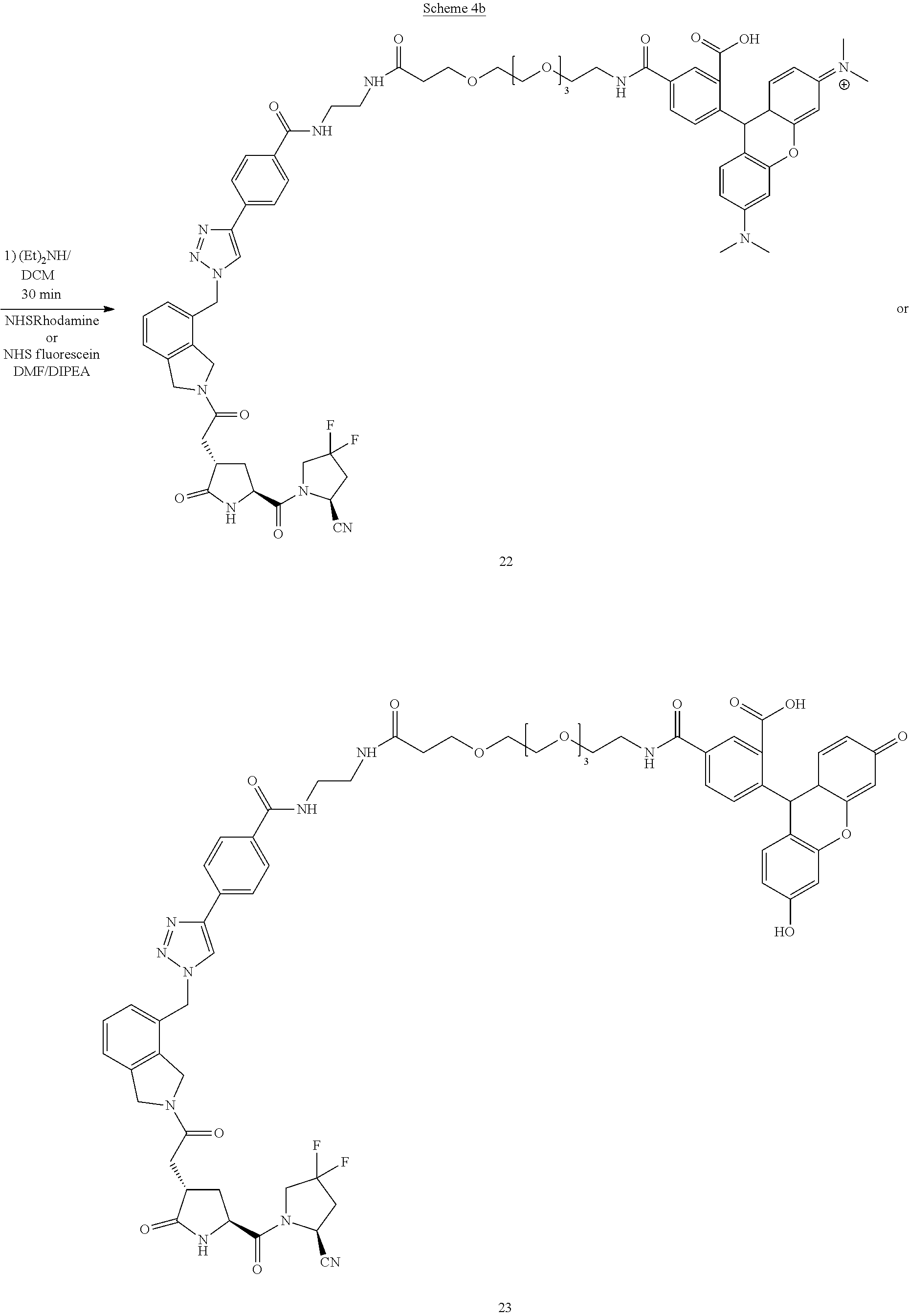

### Step-V:

Deprotection of Fmoc functionality in compounds 21a-c followed the same procedure as provided in Example 4, Step-Ill. The free amine obtained from compound 21a was treated with NHS rhodamine (1.1 eq) or 1.1 eq of NHS FITC in DMF in the presence of DIPEA (1.1 eq) for 30 min followed by purified UHPLC (A=20 Mm ammonium acetate buffer (pH=7), B=acetonitrile, solvent gradients 5% B to 95% in 60 min provided the targeted rhodamine compound 22 or FITC (23) conjugates, respectively, in quantitate yield. LCMS for compound 22: LC/MS (m/z): [M+H] calcd for: C₆₈H₇₈F₂N₁₂O₁₃ found 1308.57 g/mol. LCMS for compound 23: LC/MS (m/z): [M+H] calcd for: C₆₄H₆₇F₂N₁₀O₁₅ found: 1253.47 g/mol.

### Example 5

Sodium 2-((E)-2-((E)-2-(4-(1-(4-(1-((2-(2-((3S,5S)-5-((S)-2-cyano-4,4-difluoropyrrolidine-1-carbonyl)-2-oxopyrrolidin-3-yl)acetyl)isoindolin-4-yl)methyl)-1H-1,2,3-triazol-4-yl)phenyl)-1,6,21-trioxo-9,12,14,17-tetraoxa-2,5,20-triazatricosan-23-yl)phenoxy)-3-(2-((E)-3,3-dimethyl-5-sulfonato-1-(4-sulfonatobutyl)indolin-2-ylidene)ethylidene)cyclohex-1-en-1-yl)vinyl)-3,3-dimethyl-1-(4-sulfonatobutyl)-3H-indol-1-ium-5-sulfonate was synthesized according to Scheme 5.

### Synthesis of sodium 2-((E)-2-((E)-2-(4-(1-(4-(1-((2-(2-((3S,5S)-5-((S)-2-cyano-4,4-difluoropyrrolidine-1-carbonyl)-2-oxopyrrolidin-3-yl)acetyl)isoindolin-4-yl)methyl)-1H-1,2,3-triazol-4-yl)phenyl)-1,6,21-trioxo-9,12,14,17-tetraoxa-2,5,20-triazatricosan-23-yl)phenoxy)-3-(2-((E)-3,3-dimethyl-5-sulfonato-1-(4-sulfonatobutyl)indolin-2-ylidene)ethylidene)cyclohex-1-en-1-yl)vinyl)-3,3-dimethyl-1-(4-sulfonatobutyl)-3H-indol-1-ium-5-sulfonate

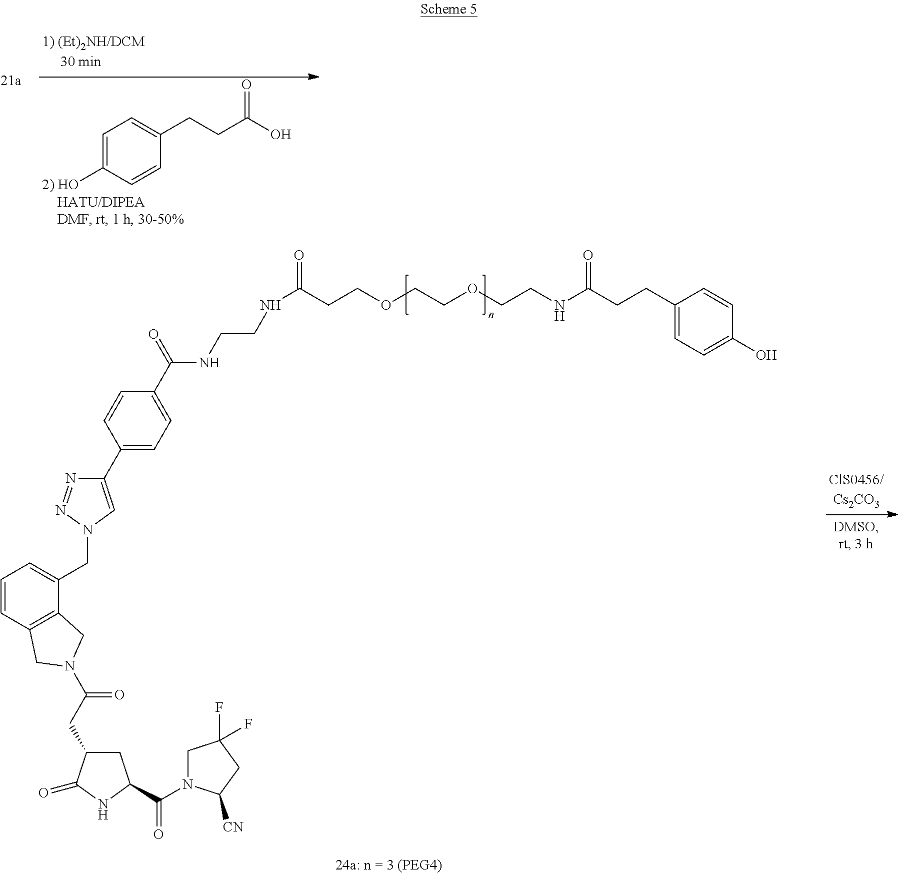

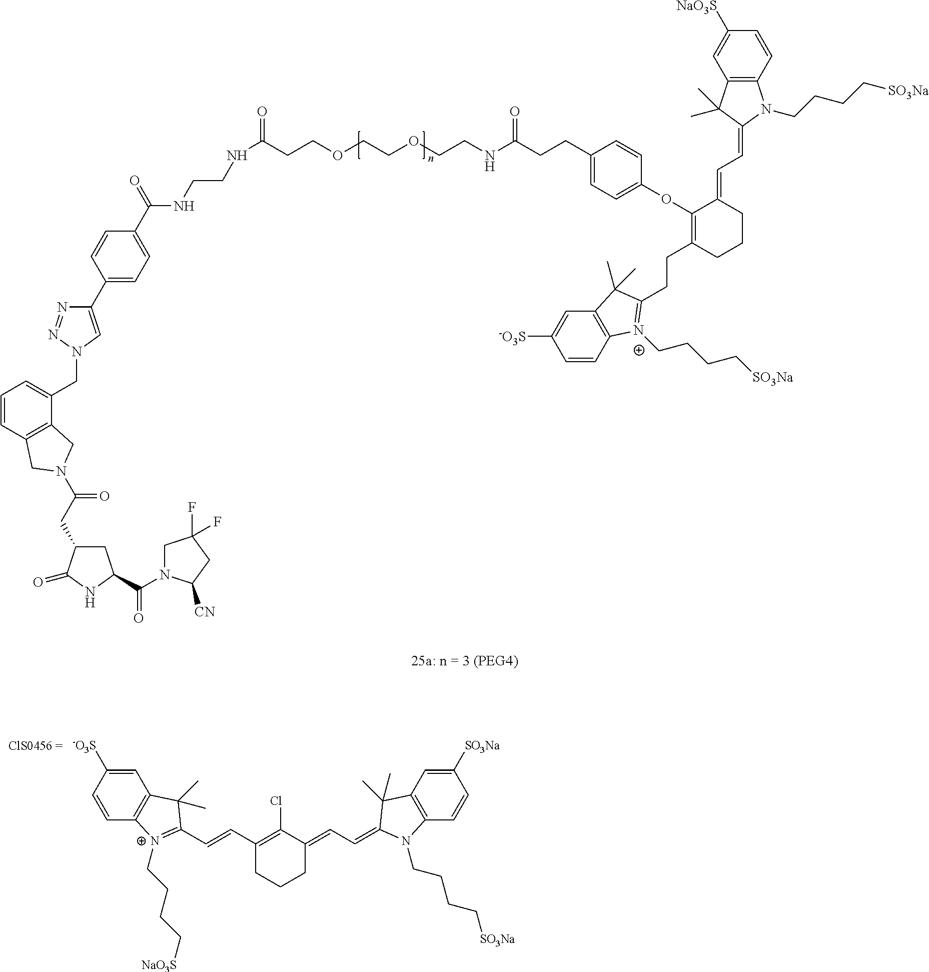

### Step-I:

The amine (1.0 eq) obtained from reducing compound 21a with diethyl amine, as provided in Example 4, Step-Ill, was dissolved in DMF. 3-(4-hydroxyphenyl)propionic acid (1.2 eq), HATU (1.3 eq) and DIPEA (3.0 eq) were added, and the reaction mixture was stirred at room temperature for 2 h to 3 h followed by evaporation under reduced pressure. The obtained crude residues were purified by using UHPLC (A=20 Mm ammonium acetate buffer (pH=7), B=acetonitrile, solvent gradients 5% B to 95% in 60 min to provide the desired compound 24a. LRMS-LC/MS (m/z): [M+H] calcd for C₅₀H₅₉F₂N₁₀O₁₀, found: 997.43.

### Step-II

To a stirred solution of compound 24a in anhydrous DMSO at room temperature under argon atmosphere was added ClS0456 Dye (1.0 eq) followed by Cs₂CO₃ (5.0 eq) and stirring was continued for an additional 3 h to 4 h, with progress of the reaction monitored by LCMS. Reaction mixture was diluted with water and purified by using UHPLC (A=20 Mm ammonium acetate buffer (pH=7), B=0 acetonitrile, solvent gradients 5% B to 35% in 60 min provided sodium 2-((E)-2-((E)-2-(4-(1-(4-(1-((2-(2-((3S,5S)-5-((S)-2-cyano-4,4-difluoropyrrolidine-1-carbonyl)-2-oxopyrrolidin-3-yl)acetyl)isoindolin-4-yl)methyl)-1H-1,2,3-triazol-4-yl)phenyl)-1,6,21-trioxo-9,12,14,17-tetraoxa-2,5,20-triazatricosan-23-yl)phenoxy)-3-(2-((E)-3,3-dimethyl-5-sulfonato-1-(4-sulfonatobutyl)indolin-2-ylidene)ethylidene)cyclohex-1-en-1-yl)vinyl)-3,3-dimethyl-1-(4-sulfonatobutyl)-3H-indol-1-ium-5-sulfonate (compound 25a). LC-MS for 25a: LC/MS (m/z): [M+H] calcd for C₉₀H₁₀₅F₂N₁₂Na₃O₂₃S₄: 1957.60.

### Example 6

4-((2-(4-(1-((2-(2-((3S,5S)-5-((S)-2-cyano-4,4-difluoropyrrolidine-1-carbonyl)-2-oxopyrrolidin-3-yl)acetyl)isoindolin-4-yl)methyl)-1H-1,2,3-triazol-4-yl)benzamido)ethyl)amino)-4-oxobutanoic acid was synthesized according to Scheme 6.

### Synthesis of 4-((2-(4-(1-((2-(2-((3S,5S)-5-((S)-2-cyano-4,4-difluoropyrrolidine-1-carbonyl)-2-oxopyrrolidin-3-yl)acetyl)isoindolin-4-yl)methyl)-1H-1,2,3-triazol-4-yl)benzamido)ethyl)amino)-4-oxobutanoic acid

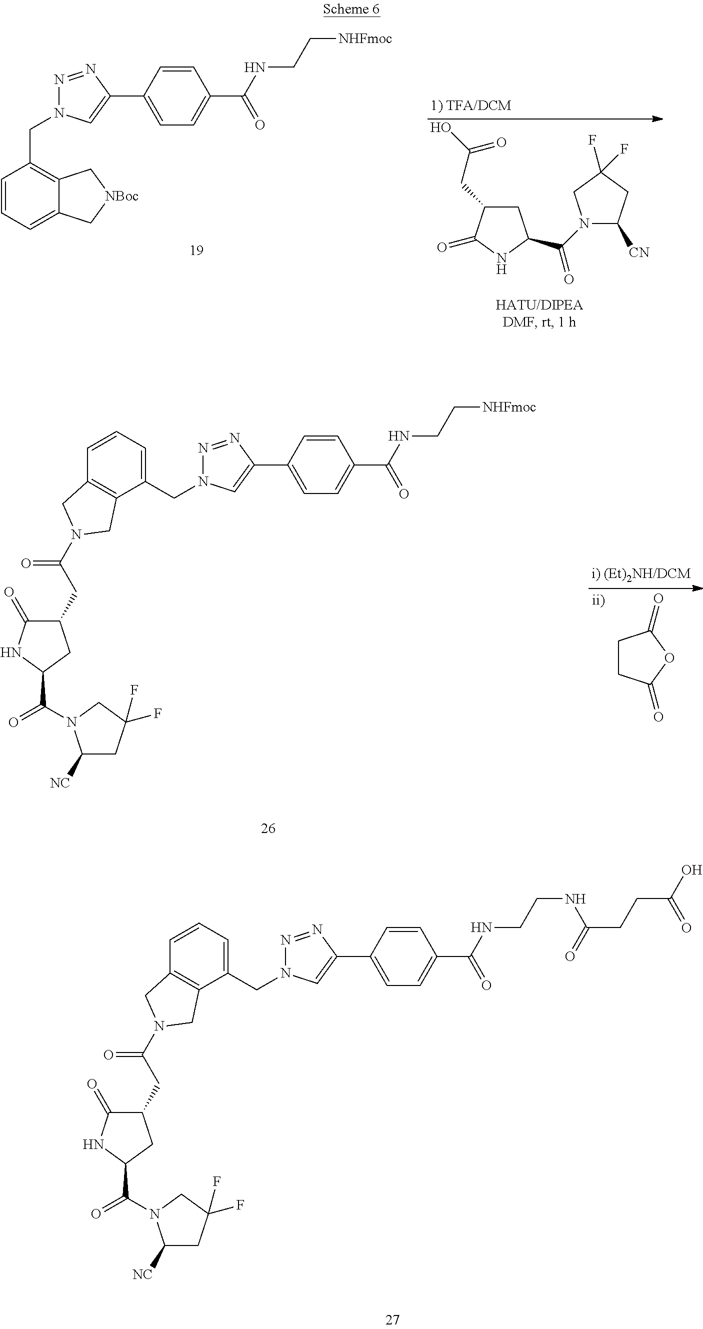

### Step-I

The synthesis of (9H-fluoren-9-yl)methyl (2-(4-(1-((2-(2-((3S,5S)-5-((S)-2-cyano-4,4-difluoropyrrolidine-1-carbonyl)-2-oxopyrrolidin-3-yl)acetyl)isoindolin-4-yl)methyl)-1H-1,2,3-triazol-4-yl)benzamido)ethyl)carbamate (compound 26) followed a similar procedure as that provided for Example 4, Step-IV (LC/MS (m/z): [M+H] calcd for: C₄₇H₄₄F₂N₉O₆ found: 868. 33). The free amine obtained from reducing compound 26 using a similar procedure provided in Example 4, Step-III was dissolved in DCM followed by addition of succinic anhydride (1.5 eq) and DIPEA (2.0 eq). The solution was stirred at room temperature for 1 h. The reaction mixture was evaporated and the obtained crude residue was purified by UHPLC (A=20 Mm ammonium acetate buffer (pH=7), B=acetonitrile, solvent gradients 5% B to 95% in 60 min to provide 4-((2-(4-(1-((2-(2-((3S,5S)-5-((S)-2-cyano-4,4-difluoropyrrolidine-1-carbonyl)-2-oxopyrrolidin-3-yl)acetyl)isoindolin-4-yl)methyl)-1H-1,2,3-triazol-4-yl)benzamido)ethyl)amino)-4-oxobutanoic acid (compound 27). LC/MS (m/z): [M+H] calcd for: C₃₆H₃₈F₂N₉O₇ found: 746.28.

### Example 7

Sodium 2-((E)-2-((E)-2-(4-(1-(2-(2-((3S,5S)-5-((S)-2-cyano-4,4-difluoropyrrolidine-1-carbonyl)-2-oxopyrrolidin-3-yl)acetyl)isoindolin-4-yl)-3,25-dioxo-6,9,12,15,18,21-hexaoxa-2,24-diazaheptacosan-27-yl)phenoxy)-3-(2-((E)-3,3-dimethyl-5-sulfonato-1-(4-sulfonatobutyl)indolin-2-ylidene)ethylidene)cyclohex-1-en-1-yl)vinyl)-3,3-dimethyl-1-(4-sulfonatobutyl)-3H-indol-1-ium-5-sulfonate was synthesized according to Scheme 7.

### Synthesis of sodium 2-((E)-2-((E)-2-(4-(1-(2-(2-((3S,5S)-5-((S)-2-cyano-4,4-difluoropyrrolidine-1-carbonyl)-2-oxopyrrolidin-3-yl)acetyl)isoindolin-4-yl)-3,25-dioxo-6,9,12,15,18,21-hexaoxa-2,24-diazaheptacosan-27-yl)phenoxy)-3-(2-((E)-3,3-dimethyl-5-sulfonato-1-(4-sulfonatobutyl)indolin-2-ylidene)ethylidene)cyclohex-1-en-1-yl)vinyl)-3,3-dimethyl-1-(4-sulfonatobutyl)-3H-indol-1-ium-5-sulfonate

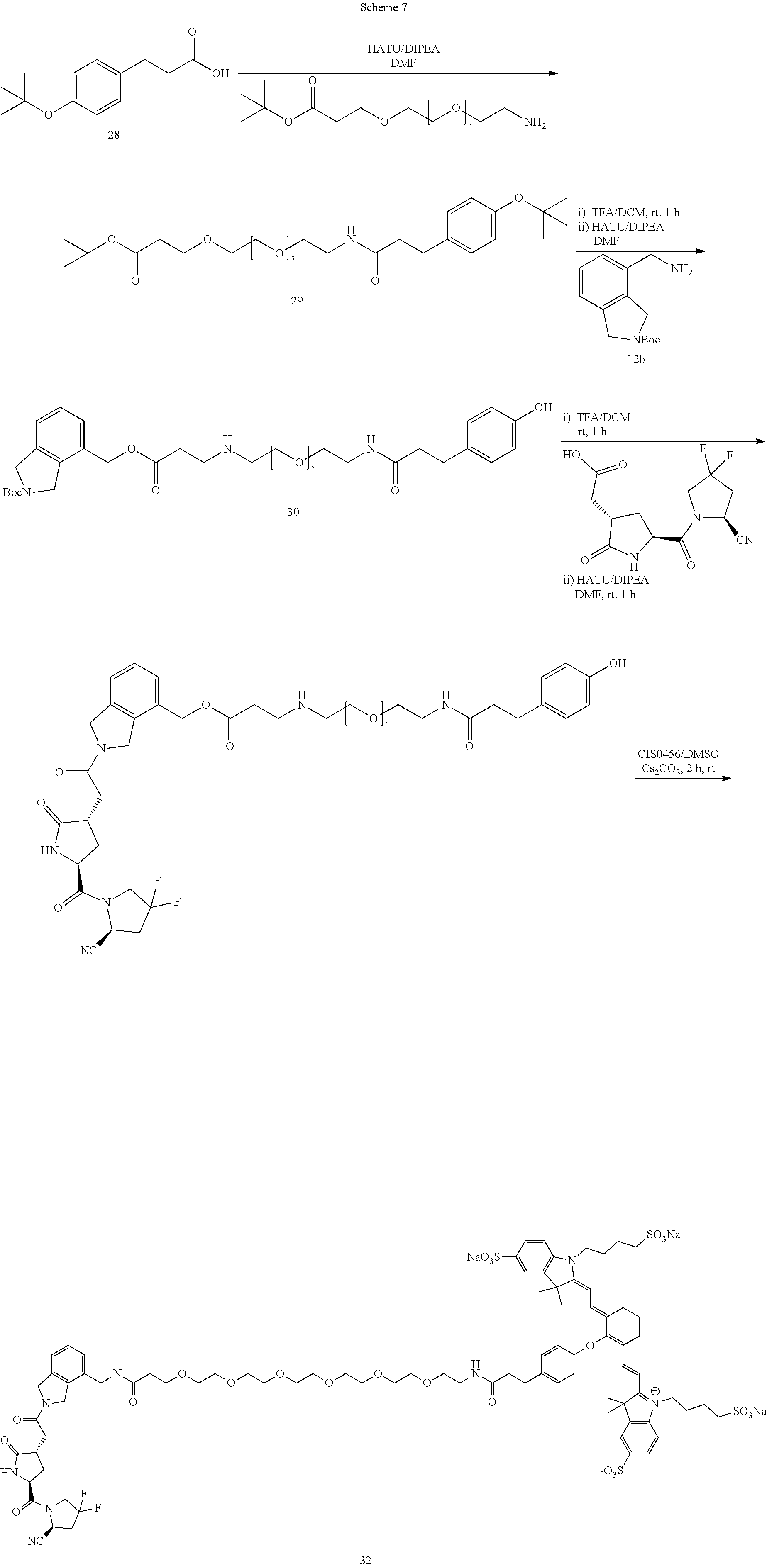

### Step-1:

To a stirred solution of 3-(4-(tert-butoxy)phenyl)propanoic acid (compound 28) in dry DMF was added HATU (1.3 eq) and DIPEA (3.0 eq) followed by amino-PEG6-t-butyl ester (1.2 eq). The reaction mixture was diluted with water, then extracted into ethyl acetate (2×20 mL). The organic layer was dried over anhydrous sodium sulphate, filtered, and the filtrate was evaporated under reduced pressure. The crude residue was purified by using combi flash using methanol and dichloromethane as mobile phase to provide tert-butyl 3-(2-(2-(3-(4-(tert-butoxy)phenyl)propanamido)ethoxy)ethoxy)propanoate (compound 29) as gummy liquid. LC/MS (m/z): [M+H]: calcd for: C₃₂H₅₆NO₁₀ found: 614.38.

### Step-II:

The tert-butyl 3-(2-(2-(3-(4-(tert-butoxy)phenyl)propanamido)ethoxy)ethoxy)propanoate (compound 29) was dissolved in DCM. Trifluoroacetic anhydride was added, and the reaction mixture was stirred at room temperature for 2 h. The reaction mixture was evaporated under reduced pressure and the obtained crude residue was dissolved in DMF. DIPEA (5.0 eq) and HATU (1.3 eq) were added followed by addition of tert-butyl 4-(aminomethyl)isoindoline-2-carboxylate (compound 12b, 1.1 eq). The mixture was stirred for 1 h. The reaction mixture was diluted with water and extracted into ethyl acetate (2×20 mL), the organic layer was evaporated, and the crude residue was purified by using combi flash using methanol and dichloromethane as mobile phase to provide the tert-butyl 4-(27-(4-hydroxyphenyl)-3,25-dioxo-6,9,12,15,18,21-hexaoxa-2,24-diazaheptacosyl)isoindoline-2-carboxylate (compound 30) as gummy liquid. LC/MS (m/z): [M+H]: calcd for C₃₈H₅₈N₃O₁₁ found: 732.40.

### Step-III & Step-IV:

N-((2-(2-((3S,5R)-5-((S)-2-cyano-4,4-difluoropyrrolidine-1-carbonyl)-2-oxopyrrolidin-3-yl)acetyl)isoindolin-4-yl)methyl)-1-(3-(4-hydroxyphenyl)propanamido)-3,6,9,12,15,18-hexaoxahenicosan-21-amide (compound 31) & sodium 2-((E)-2-((E)-2-(4-(1-(2-(2-((3S,5S)-5-((S)-2-cyano-4,4-difluoropyrrolidine-1-carbonyl)-2-oxopyrrolidin-3-yl)acetyl)isoindolin-4-yl)-3,25-dioxo-6,9,12,15,18,21-hexaoxa-2,24-diazaheptacosan-27-yl)phenoxy)-3-(2-((E)-3,3-dimethyl-5-sulfonato-1-(4-sulfonatobutyl)indolin-2-ylidene)ethylidene)cyclohex-1-en-1-yl)vinyl)-3,3-dimethyl-1-(4-sulfonatobutyl)-3H-indol-1-ium-5-sulfonate (compound 32) were synthesized using a similar procedure as that provided in Example 4, Step-IV and Example 5, Step-I, respectively. LCMS of compound 31: LC/MS (m/z): [M+H] calcd for C₄₅H₆₁F₂N₆O₁₂, found: 915.42 LCMS of compound 32: LC/MS (m/z): [M+H] calcd for: C₈₃H₁₀₄F₂N₈Na₃O₂₄S₄ found: 1831.56.

### Example 8

Sodium 2-((E)-2-((E)-2-(4-(1-(1-((2-(2-((3S,5S)-5-((S)-2-cyano-4,4-difluoropyrrolidine-1-carbonyl)-2-oxopyrrolidin-3-yl)acetyl)isoindolin-4-yl)methyl)-1H-1,2,3-triazol-4-yl)-3,19-dioxo-6,9,12,15-tetraoxa-2,18-diazahenicosan-21-yl)phenoxy)-3-(2-((E)-3,3-dimethyl-5-sulfonato-1-(4-sulfonatobutyl)indolin-2-ylidene)ethylidene)cyclohex-1-en-1-yl)vinyl)-3,3-dimethyl-1-(4-sulfonatobutyl)-3H-indol-1-ium-5-sulfonate was synthesized according to Scheme 8.

### Synthesis of sodium 2-((E)-2-((E)-2-(4-(1-(1-((2-(2-((3S,5S)-5-((S)-2-cyano-4,4-difluoropyrrolidine-1-carbonyl)-2-oxopyrrolidin-3-yl)acetyl)isoindolin-4-yl)methyl)-1H-1,2,3-triazol-4-yl)-3,19-dioxo-6,9,12,15-tetraoxa-2,18-diazahenicosan-21-yl)phenoxy)-3-(2-((E)-3,3-dimethyl-5-sulfonato-1-(4-sulfonatobutyl)indolin-2-ylidene)ethylidene)cyclohex-1-en-1-yl)vinyl)-3,3-dimethyl-1-(4-sulfonatobutyl)-3H-indol-1-ium-5-sulfonate

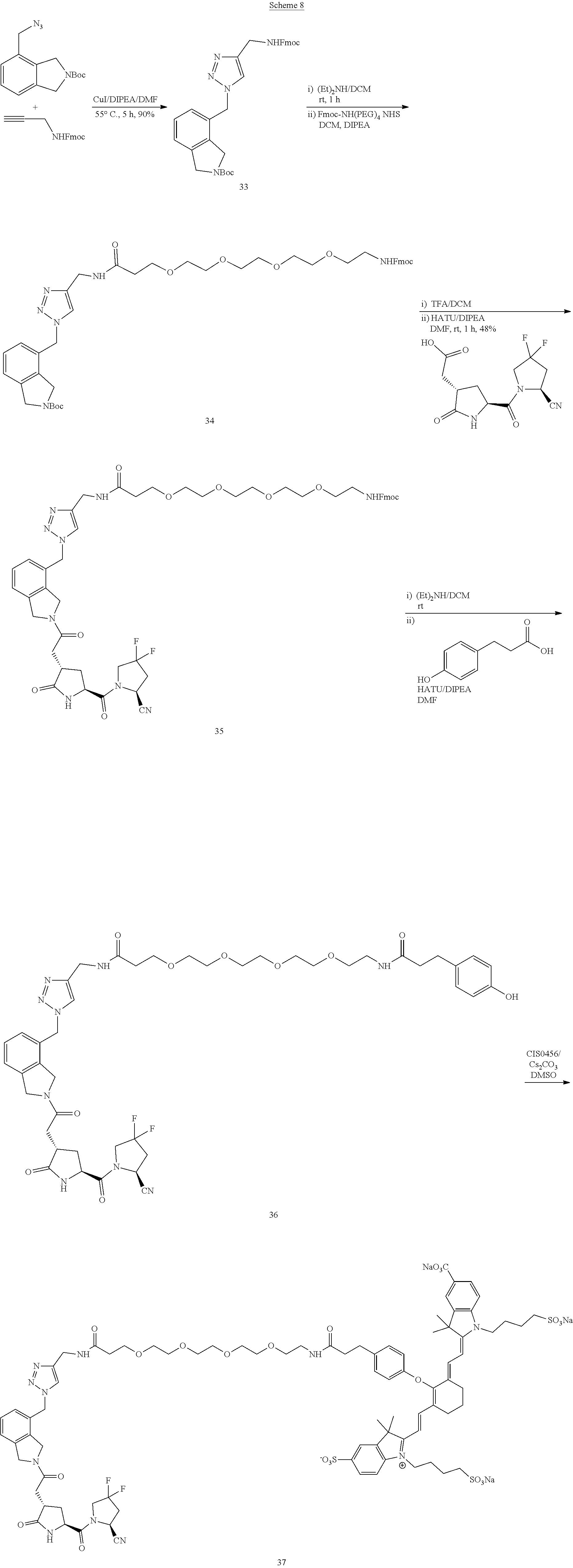

### Step-I

To a mixture of tert-butyl 4-(azidomethyl)isoindoline-2-carboxylate (compound 12a, 1.0 eq) and Fmoc propargyl amine (1.2 eq) in dry DMF (5.0 mL) was added CuI (0.5 eq) followed by DIPEA (2.0 eq). The reaction mixture was stirred at 50° C., under nitrogen atmosphere for 5 h, then the reaction mixture was cooled to room temperature, diluted with water (20 mL), and vigorously stirred for 15 min. The solid residue that formed in the reaction mixture was filtered and washed with water 2×20 mL, and dried under vacuum for 1 h to provide tert-butyl 4-((4-(((((9H-fluoren-9-yl)methoxy)carbonyl)amino)methyl)-1H-1,2,3-triazol-1-yl)methyl)isoindoline-2-carboxylate (compound 33, 96%) as solid and this was taken to further step without further purification.

To a stirred solution of compound 33 (1 mmole) in DCM (5 mL) was added (Et)₂NH (2 mL) and stirring was continued for 2 h, while progress of the reaction was monitored by LCMS. The reaction mixture was evaporated under reduced pressure, and the obtained crude residue was treated with diethyl ether. The ether layer was decanted and the solid compound was dried under vacuum followed by dissolving in DCM. To this reaction mixture Fmoc-NH(PEG4) NHS ester (1.2 eq) and DIPEA (3.0 eq) were added. The reaction mixture was stirred at room temperature for 1 h, evaporated under reduced pressure, and the crude residue was purified by using combi flash using methanol+DCM as mobile phase to provide the compound 34.

### Example 9

2-((3S,5S)-5-((2S,4S)-2-cyano-4-fluoropyrrolidine-1-carbonyl)-2-oxopyrrolidin-3-yl)acetic acid was synthesized according to Scheme 9.

### Synthesis of 2-((3S,5S)-5-((2S,4S)-2-cyano-4-fluoropyrrolidine-1-carbonyl)-2-oxopyrrolidin-3-yl)acetic acid

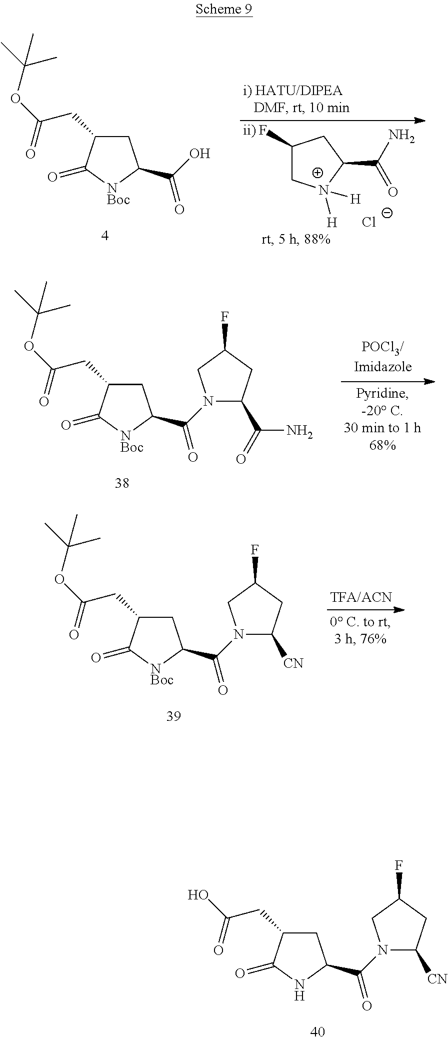

### Step-I:

To a stirred solution of (2S,4S)-4-(2-(tert-butoxy)-2-oxoethyl)-1-(tert-butoxycarbonyl)-5-oxopyrrolidine-2-carboxylic acid (compound 4, 1.0 eq) in dry DMF was added HATU (1.3), DIPEA (3.0 eq) and stirring was continued for 10 min for activation of the acid functionality in 4. 4-Cis-fluoro-L-prolinamide hydrochloride (1.2) and DIPEA (2.0 eq) were added to the mixture and stirring was continued at room temperature under nitrogen atmosphere for 5 h. The reaction mixture was diluted with water and brine and extracted into ethyl acetate. The combined organic extracts were dried over anhydrous sodium sulphate and filtered. The filtrate was evaporated under reduced pressure and the obtained crude residue was purified by combi flash using DCM/MeOH as mobile phase to provide tert-butyl (3S,5S)-3-(2-(tert-butoxy)-2-oxoethyl)-5-((2S,4S)-2-carbamoyl-4-fluoropyrrolidine-1-carbonyl)-2-oxopyrrolidine-1-carboxylate (compound 38) as gummy solid.

### Step-II:

To a mixture of tert-butyl (3S,5S)-3-(2-(tert-butoxy)-2-oxoethyl)-5-((2S,4S)-2-carbamoyl-4-fluoropyrrolidine-1-carbonyl)-2-oxopyrrolidine-1-carboxylate (compound 38, 1.0 eq) and imidazole (1.2 eq.) in pyridine cooled to −20° C. was added phosphoryl chloride (POCl₃) (1.0 eq) under nitrogen. After being stirred for 30 min to 1 h at −20° C., the mixture was evaporated to dryness in vacuo. The resulting brown solid was dissolved in CH₂Cl₂ (40 mL) and washed with 1.0 N aqueous citric acid (40 mL). The organic phase was dried over magnesium sulphate, filtered, and concentrated under reduced pressure to yield the crude material as a viscous oil. The crude material was purified by combi flash (eluted with hexane/EtOAc) to give tert-butyl (3S,5S)-3-(2-(tert-butoxy)-2-oxoethyl)-5-((2S,4S)-2-cyano-4-fluoropyrrolidine-1-carbonyl)-2-oxopyrrolidine-1-carboxylate (compound 39) as a white solid.

### Step-III:

To a solution of tert-butyl (3S,5S)-3-(2-(tert-butoxy)-2-oxoethyl)-5-((2S,4S)-2-cyano-4-fluoropyrrolidine-1-carbonyl)-2-oxopyrrolidine-1-carboxylate (compound 39, 1 mmol) in CH₃CN (5 mL) at 0° C. was added TFA (5 mL) dropwise over 5 min. The reaction mixture was stirred for 1 h at room temperature. The reaction mixture was concentrated under vacuum and crystallized with EA/ether to yield 2-((3S,5S)-5-((2S,4S)-2-cyano-4-fluoropyrrolidine-1-carbonyl)-2-oxopyrrolidin-3-yl)acetic acid (compound 40) as a white powder. LC/MS (m/z): calcd for C₁₂H₁₅FN₃O₄ found 284.10.

### Example 10

4-((2-(4-(1-((2-(2-((3S,5S)-5-((2S,4S)-2-cyano-4-fluoropyrrolidine-1-carbonyl)-2-oxopyrrolidin-3-yl)acetyl)isoindolin-4-yl)methyl)-1H-1,2,3-triazol-4-yl)benzamido)ethyl)amino)-4-oxobutanoic acid was synthesized according to Scheme 10.

### Synthesis of 4-((2-(4-(1-((2-(2-((3S,5S)-5-((2S,4S)-2-cyano-4-fluoropyrrolidine-1-carbonyl)-2-oxopyrrolidin-3-yl)acetyl)isoindolin-4-yl)methyl)-1H-1,2,3-triazol-4-yl)benzamido)ethyl)amino)-4-oxobutanoic acid

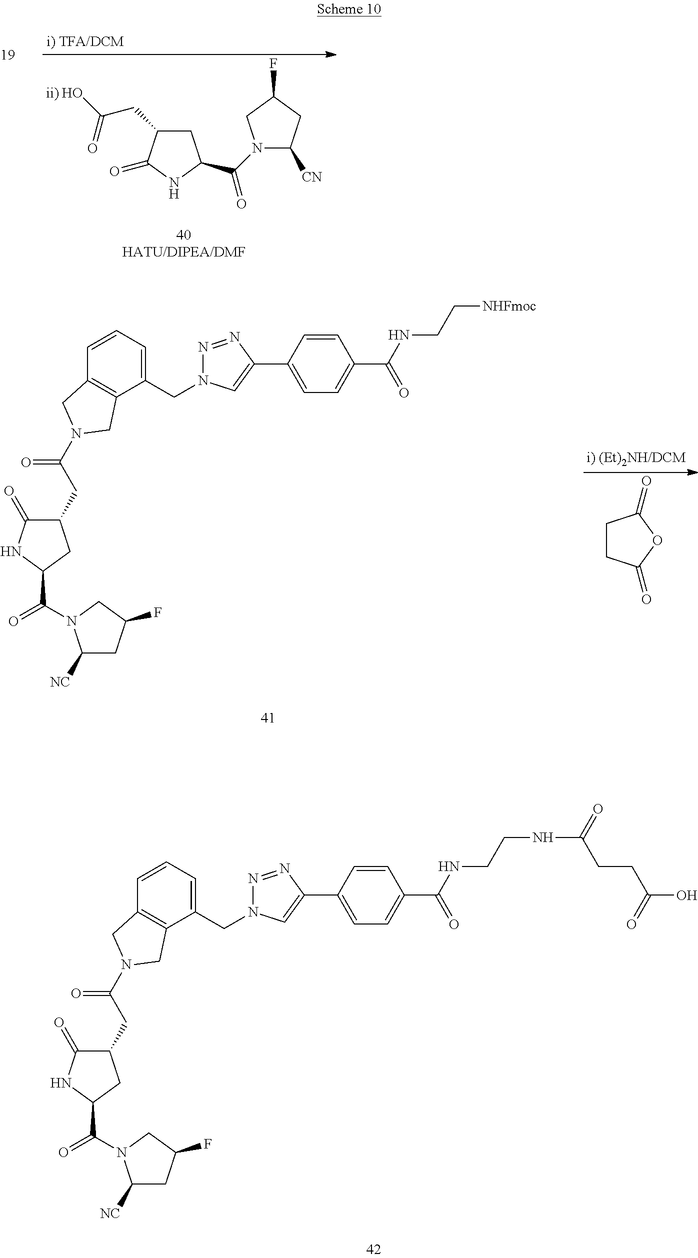

TFA (10 eq) was added to a stirred solution of compound 19 (1.0 eq) in DMF (1.0 mL) at room temperature, and stirring was continued for 30 min. The reaction mixture was evaporated and dried under vacuum. In a separate round bottom flask, compound 40 (1.2 eq) was dissolved in DMF (0.5 mL) followed by HATU (1.3 eq) and DIPEA (5.0 eq), and the reaction mixture was stirred under nitrogen atmosphere at room temperature for 10 min for activation of the acid functionality in compound 40. The amines obtained from compound 19 were dissolved in DMF (1 mL) and added to the above reaction mixture and stirring was continued for an additional 2 h. The reaction mixture was diluted with water (15 mL) and extracted into ethyl acetate (2×15 mL). Organic extracts were dried over anhydrous sodium sulphate and concentrated, and the obtained crude residue was purified by combi flash using DCM and MeOH as mobile phase to give the desired compound 41. LCMS of compound 41: LC/MS (m/z): [M+H] calcd for C₄₇H₄₅FN₉O₆ found: 850.34. LCMS of compound 42: LC/MS (m/z): [M+H] calcd for C₃₆H₄₀FN₉O₇ found 728.29 g/mol.

### Example 11

5-((2-(2-(2-((3S,5S)-5-((S)-2-cyano-4,4-difluoropyrrolidine-1-carbonyl)-2-oxopyrrolidin-3-yl)acetyl)isoindoline-4-carboxamido)ethyl)carbamoyl)-2-(6-(dimethylamino)-3-(dimethyliminio)-3H-xanthen-9-yl)benzoate was synthesized according to Scheme 11.

### Synthesis of 5-((2-(2-(2-((3S,5S)-5-((S)-2-cyano-4,4-difluoropyrrolidine-1-carbonyl)-2-oxopyrrolidin-3-yl)acetyl)isoindoline-4-carboxamido)ethyl)carbamoyl)-2-(6-(dimethylamino)-3-(dimethyliminio)-3H-xanthen-9-yl)benzoate

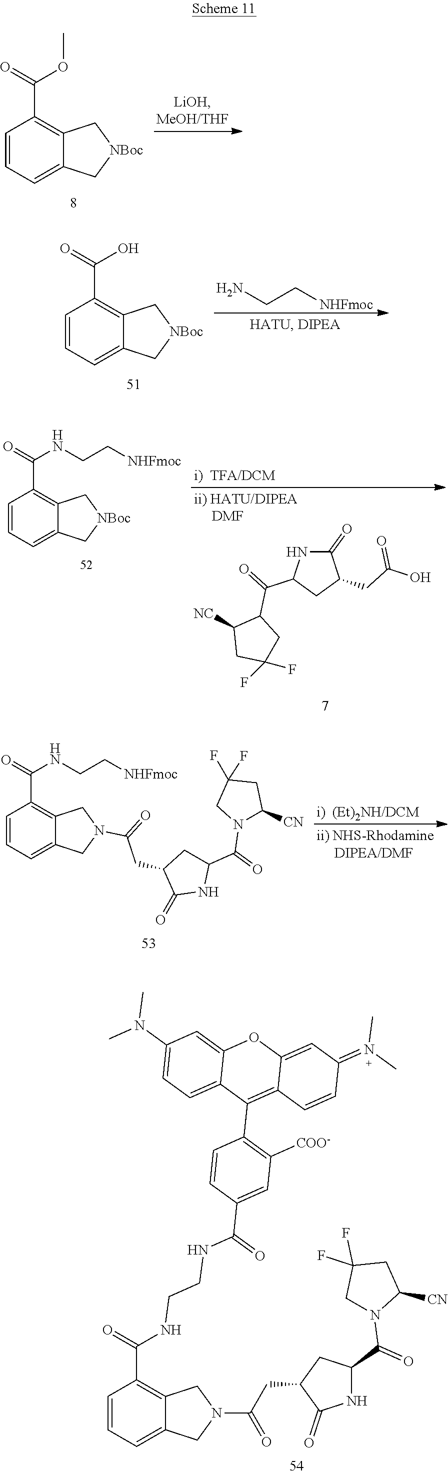

### Step-I

LiOH (172.8 mg, 7.2 mmol, 10 eq) was added to a stirred solution of 2-(tert-butyl) 4-methyl isoindoline-2,4-dicarboxylate (Compound 8, 200 mg, 0.72 mmol) in MeOH/THF/H₂O (0.2 mL/0.6 mL/0.2 mL). The mixture was stirred for 5 h. The solvent was removed under reduced pressure, and the mixture was dissolved in water (1 mL), adjusting the pH to 7 with citric acid (1 M). The product was extracted with EA (3 mL×3). The organic layers were combined, dried with sodium sulfate, and concentrated under reduced pressure to provide 2-(tert-butoxycarbonyl)isoindoline-4-carboxylic acid (Compound 51), which was used in the next step without further purification.

### Step-II

HATU (456 mg, 1.2 mmol, 1.2 eq) and DIPEA (258 mg, 2 mmol, 2.0 eq) were added to a solution of 2-(tert-butoxycarbonyl)isoindoline-4-carboxylic acid (Compound 51, 263 mg, 1.0 mmol, 1.0 eq) in DMF (5 mL). The mixture was stirred for 10 min, and then (9H-fluoren-9-yl)methyl (2-aminoethyl)carbamate hydrochloride salt (350.9 mg, 1.1 mmol, 1.1 eq) was added. The mixture was diluted with ethyl acetate (2 mL) and washed with H₂O (2 mL×3). The organic layers were combined, dried with sodium sulfate, and concentrated under reduced pressure. The remaining oil was purified with combi flash chromatography using hexane/ethyl acetate as eluent, providing tert-butyl 4-((2-((((9H-fluoren-9-yl)methoxy)carbonyl)amino)ethyl)carbamoyl)isoindoline-2-carboxylate (Compound 52) in 352.5 mg yield as white solid.

### Step-III

TFA (10 eq) was added to a stirred solution of compound 52 (1.0 eq) in DMF (1.0 mL) at room temperature, and stirring was continued for 30 min. The reaction mixture was evaporated and dried under vacuum. In a separate round bottom flask, acid Compound 7 (1.2 eq) was dissolved in DMF (0.5 mL) followed by HATU (1.3 eq) and DIPEA (5.0 eq) and the reaction mixture was stirred under nitrogen atmosphere at room temperature for 10 min for activation of acid functionality in compound 7. The amines obtained from compound 52 were dissolved in DMF (1 mL), added to the above reaction mixture, and stirring was continued for an additional 2 h. The reaction mixture was diluted with water (15 mL) and extracted into ethyl acetate (2×15 mL). The organic extracts were dried over anhydrous sodium sulphate. The extract was concentrated and the obtained crude residue was purified by combiflash using DCM and MeOH as mobile phase to give the desired compound 53. (Et)₂NH was added to a solution of compound 53 DCM and stirred at room temperature for 1 h, evaporated under reduced pressure, and the crude reside was treated with diethyl ether, dried and taken to the next step without purification.

N-(2-aminoethyl)-2-(2-((3S)-5-((S)-2-cyano-4,4-difluoropyrrolidine-1-carbonyl)-2-oxopyrrolidin-3-yl)acetyl)isoindoline-4-carboxamide (10.0 mg, 0.02 mmol, 1.0 eq) was dissolved in DMF (1 mL), then rhodamine-NHS (12.9 mg, 0.024 mmol, 1.2 eq) was added followed by DIPEA (3.87 mg, 0.03 mmol, 1.5 eq). The mixture was stirred at room temperature for 2 h. The reaction mixture was diluted with water and purified by using UHPLC (A=20 Mm ammonium acetate buffer (pH=7), B=acetonitrile, solvent gradients 5% B to 95% in 60 min to provide 5-((2-(2-(2-((3S,5S)-5-((S)-2-cyano-4,4-difluoropyrrolidine-1-carbonyl)-2-oxopyrrolidin-3-yl)acetyl)isoindoline-4-carboxamido)ethyl)carbamoyl)-2-(6-(dimethylamino)-3-(dimethyliminio)-3H-xanthen-9-yl)benzoate (Compound 54). LC/MS (m/z): [M+H] calcd for C₄₈H₄₆F₂N₈O₈, found 901.3 g/mol.

### Example 12

5-((2-(3-(2-(2-((3S,5S)-5-((S)-2-cyano-4,4-difluoropyrrolidine-1-carbonyl)-2-oxopyrrolidin-3-yl)acetyl)isoindolin-4-yl)propanamido)ethyl)carbamoyl)-2-(6-(dimethylamino)-3-(dimethyliminio)-3H-xanthen-9-yl)benzoate was synthesized according to Scheme 12.

### Synthesis of 5-((2-(3-(2-(2-((3S,5S)-5-((S)-2-cyano-4,4-difluoropyrrolidine-1-carbonyl)-2-oxopyrrolidin-3-yl)acetyl)isoindolin-4-yl)propanamido)ethyl)carbamoyl)-2-(6-(dimethylamino)-3-(dimethyliminio)-3H-xanthen-9-yl)benzoate

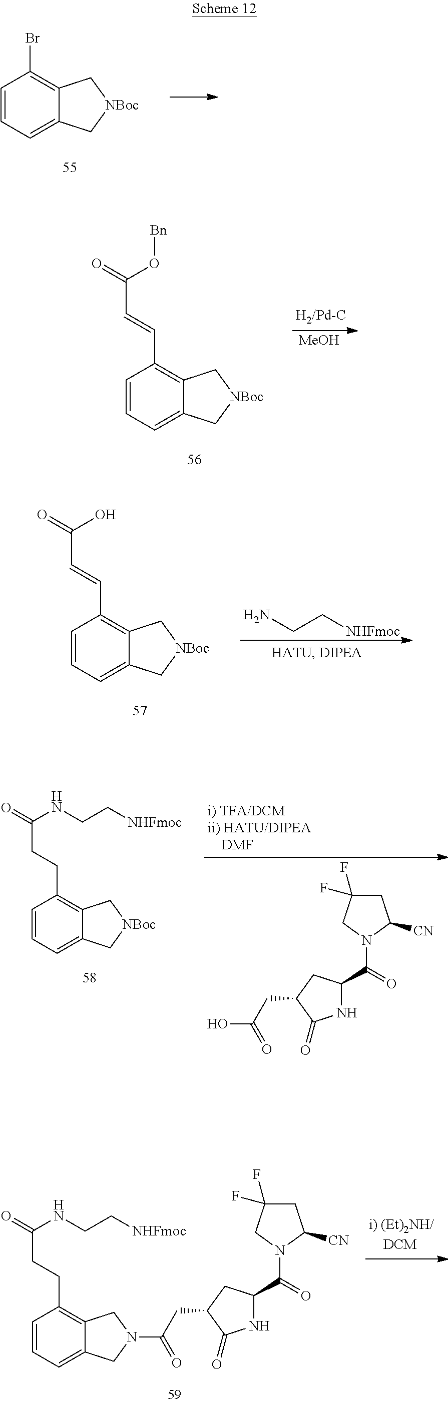

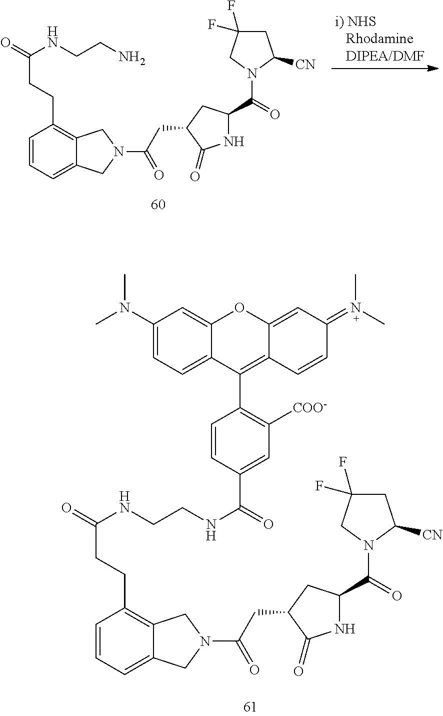

### Step-I

To a solution of tert-butyl 4-bromoisoindoline-2-carboxylate (compound 55) (297 mg, 1 mmol, 1.0 eq) under argon atmosphere in DMF (1 mL) was added benzyl acrylate (486 mg, 3 mmol, 3.0 eq), Pd(OAc)₂ (22.3 mg, 0.1 mmol, 0.1 eq), P(o-Tol)₃ (60.8 mg, 0.2 mmol, 0.2 eq) and DIPEA (387 mg, 3.0 mmol, 3.0 eq). The resulting mixture was heated at 100° C. for 8 h. After completion, the reaction was quenched with water (3 mL). The aqueous layer was extracted with EA (10 mL×3), and the organic phases were combined and dried with sodium sulfate. The organic layers were concentrated under reduced pressure, and the residue was purified with combi flash chromatography using hexane/ethyl acetate as eluent, producing tert-butyl (E)-4-(3-(benzyloxy)-3-oxoprop-1-en-1-yl)isoindoline-2-carboxylate (compound 56) in 148 mg as yellowish oil without further purification. Compound 56 was hydrogenated using Pd/C in MeOH under hydrogen atmosphere for 8 h to provide 3-(2-(tert-butoxycarbonyl)isoindolin-4-yl)propanoic acid (compound 57). LC/MS for 57: LC/MS (m/z) [M+H] calcd for C₁₆H₂₂NO₄ found 292.15.

Compounds 58-61 were synthesized in a similar manner as provided in Example 11 to produce 5-((2-(3-(2-(2-((3S,5S)-5-((S)-2-cyano-4,4-difluoropyrrolidine-1-carbonyl)-2-oxopyrrolidin-3-yl)acetyl)isoindolin-4-yl)propanamido)ethyl)carbamoyl)-2-(6-(dimethylamino)-3-(dimethyliminio)-3H-xanthen-9-yl)benzoate. LC/MS for compound 61 (m/z): [M+H] calcd for C₅₀H₅₁F₂N₈O₈, found 929.3 g/mol.

### Example 13

N-(4-((2-(2-(2-((3S,5S)-5-((S)-2-cyano-4,4-difluoropyrrolidine-1-carbonyl)-2-oxopyrrolidin-3-yl)acetyl)-2,3,7,7a-tetrahydro-1H-isoindole-4-carboxamido)ethyl)amino)-4-oxobutanoyl)-S-((2-((((5-(6-(5-((2,4-difluorophenyl)sulfonamido)-6-methoxypyridin-3-yl)quinolin-4-yl)pyridin-2-yl)methoxy)carbonyl)oxy)ethyl)thio)cysteine was synthesized according to Scheme 13.

### Synthesis of N-(4-((2-(2-(2-((3S,5S)-5-((S)-2-cyano-4,4-difluoropyrrolidine-1-carbonyl)-2-oxopyrrolidin-3-yl)acetyl)-2,3,7,7a-tetrahydro-1H-isoindole-4-carboxamido)ethyl)amino)-4-oxobutanoyl)-S-((2-((((5-(6-(5-((2,4-difluorophenyl)sulfonamido)-6-methoxypyridin-3-yl)quinolin-4-yl)pyridin-2-yl)methoxy)carbonyl)oxy)ethyl)thio)cysteine

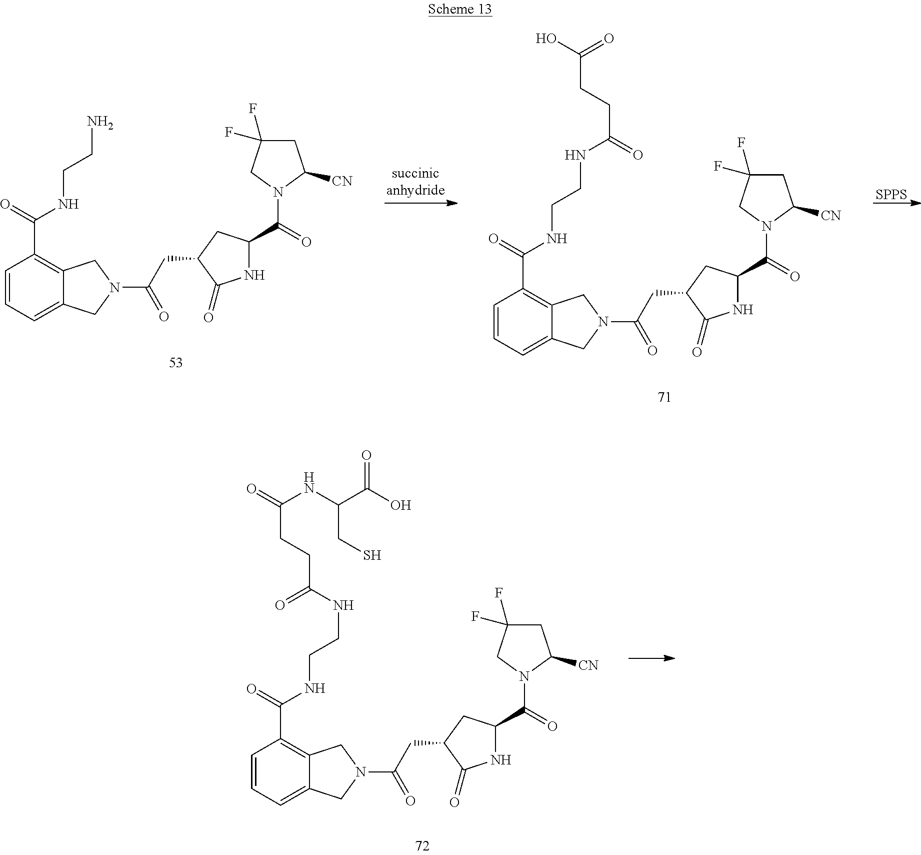

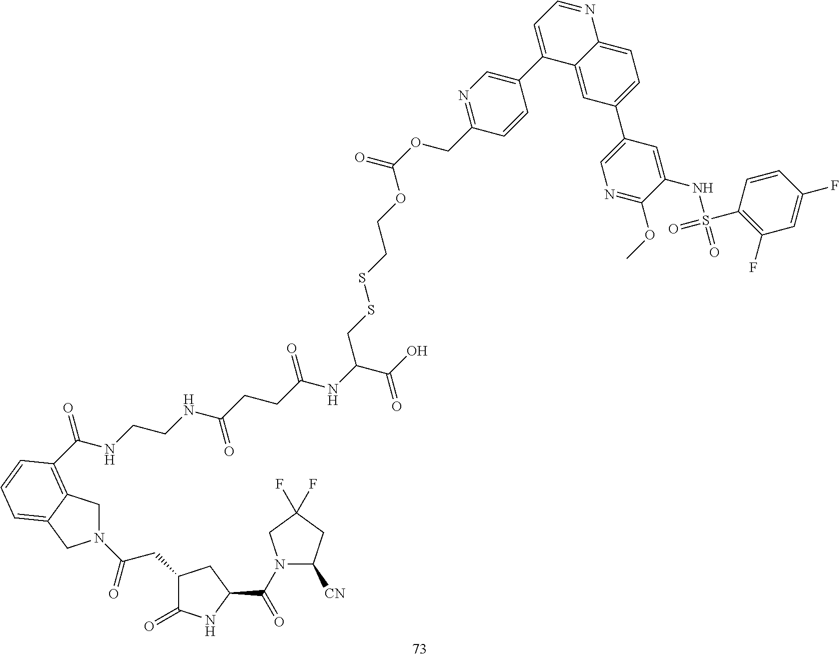 [SMILES: COc1ncc(-c2ccc3nccc(-c4ccc(COC(=O)OCCSSCC(NC(=O)CCC(=O)NCCNC(=O)c5cccc6c5CN(C(=O)C[C@@H]5C[C@@H](C(=O)N7CC(F)(F)C[C@H]7C#N)NC5=O)C6)C(=O)O)nc4)c3c2)cc1NS(=O)(=O)c1ccc(F)cc1F]

N-(2-aminoethyl)-2-(2-((3S,5S)-5-((S)-2-cyano-4,4-difluoropyrrolidine-1-carbonyl)-2-oxopyrrolidin-3-yl)acetyl)isoindoline-4-carboxamide (compound 53, 97.6 mg, 0.2 mmol, 1.0 eq) was dissolved in DCM (2 mL), then succinic anhydride (24 mg, 0.24 mmol, 1.2 eq) was added followed by DIPEA (51.6 mg, 0.4 mmol, 2 eq). The resulting mixture was kept for 8 h. The solvent was removed under reduced pressure. The residue was purified with preparative RP-HPLC [A=2 mM ammonium acetate buffer (pH 7.0), B=acetonitrile, solvent gradient 5% B to 55% B for 60 min] to produce 4-((2-(2-(2-((3S,5S)-5-((S)-2-cyano-4,4-difluoropyrrolidine-1-carbonyl)-2-oxopyrrolidin-3-yl)acetyl)isoindoline-4-carboxamido)ethyl)amino)-4-oxobutanoic acid (compound 71). LC/MS (m/z): [M+H] Exact Mass: 588.21 g/mol. 4-((2-(2-(2-((3S,5S)-5-((S)-2-cyano-4,4-difluoropyrrolidine-1-carbonyl)-2-oxopyrrolidin-3-yl)acetyl)isoindoline-4-carboxamido)ethyl)amino)-4-oxobutanoic acid (compound 71, 117.6 mg, 0.2 mmol, 1.0 eq) was introduced to solid phase peptide coupling conditions with HATU (91.2 mg, 0.24 mmol, 1.2 eq) and DIPEA (51.6 mg, 0.4 mmol, 2.0 eq) using H-Cys(Trt)-2-Cl-Trt (1.2 eq). The final product was cleaved from the resin using a cocktail solution of TFA:water:TIPS:ethanedithiol (95%: 2.5%: 2.5%: 2.5%). The crude compound was precipitated in ether to yield (4-((2-(2-(2-((3S,5S)-5-((S)-2-cyano-4,4-difluoropyrrolidine-1-carbonyl)-2-oxopyrrolidin-3-yl)acetyl)isoindoline-4-carboxamido)ethyl)amino)-4-oxobutanoyl)cysteine (compound 72). LC/MS (m/z): [M+H] Exact Mass: 691.22. (5-(6-(5-((2,4-difluorophenyl)sulfonamido)-6-methoxypyridin-3-yl)quinolin-4-yl)pyridin-2-yl)methyl (2-mercaptoethyl) carbonate (7.46 mg, 0.01 mmol) and (4-((2-(2-(2-((3S,5S)-5-((S)-2-cyano-4,4-difluoropyrrolidine-1-carbonyl)-2-oxopyrrolidin-3-yl)acetyl)isoindoline-4-carboxamido)ethyl)amino)-4-oxobutanoyl)cysteine (compound 72, 6.91 mg, 0.01 mmol) were dissolved in DMF (1 mL) and stirred. Following completion of the reaction, crude product was purified by preparative RP-HPLC [A=2 mM ammonium acetate buffer (pH 7.0), B=acetonitrile, solvent gradient 5% B to 75% B for 60 min] to yield N-(4-((2-(2-(2-((3S,5S)-5-((S)-2-cyano-4,4-difluoropyrrolidine-1-carbonyl)-2-oxopyrrolidin-3-yl)acetyl)-2,3,7,7a-tetrahydro-1H-isoindole-4-carboxamido)ethyl)amino)-4-oxobutanoyl)-S-((2-((((5-(6-(5-((2,4-difluorophenyl)sulfonamido)-6-methoxypyridin-3-yl)quinolin-4-yl)pyridin-2-yl)methoxy)carbonyl)oxy)ethyl)thio)cysteine (compound 73). LRMS-LCMS (m/z): [M+H]+ calculated for C₆₀H₅₈F₄N₁₁O₁₄S₃, 1328.3; found 1328.2).

### Cell Culture

FaDu, HT29, MDA-MB231, KB,4T1 PANC1, U87MG, LaNCap and human FAP-transfected HEK-FAP and HT1080-FAP cells were cultured in a medium consisting of RPMI 1640, DMEM and EMEM, 10% FBS, 1% penicillin-streptomycin, 1% 2 mM glutamine at 37° C. in a 5% CO₂ and 95% humidified atmosphere. The cells used in this study were initiated by thawing frozen vials from a master stock saved from the original cell lines purchased from ATCC. All the experiments were performed within two to five passages following thawing of the cells. No mycoplasma test was performed for any of the cell lines.

### Animal Husbandry

5-6 weeks old female athymic nu/nu mice were purchased from Harlan Laboratories and allowed access to normal rodent chow and water ad libitum. The animals were maintained on a standard 12 h light-dark cycle. All animal procedures were approved by the Purdue Animal Care and Use Committee.

### Confocal Binding Studies of FAP-Targeting Ligand

Method 1: HT1080-FAP cells (1000000 cells/well) were seeded in 4-well confocal plates. The cells were allowed to grow as a monolayer over 24 h at 37° C. and were incubated with various concentrations of conjugate ranging from 3.0 nM (lowest) to 25 nM (highest) in 1% FBS in PBS for 1 h at 37° C. Cells were washed with 1% FBS (3×500 μL) and the cells were left in 500 μl of 1% FBS and images were acquired with confocal microscopy. Again, the PBS in cells was replaced with growth media and the cells were re-incubated at 37° C. for 8 to 48 h. Images acquired at different concentrations of the compound at 37° C. are shown in FIG. 3, at different time points are shown in FIG. 4, and with 100-fold excess of competition ligand are shown in FIG. 5.

Method 2: Human FAP-transfected HT1080-FAP cells were (100,000) were plated on 4-well confocal plates and incubated with different concentrations (50 nM, 25 nM, 12.5 nM, 6.25 nM, 3.125 nM and 1.65 nM) of the compound for 1 h at 37° C. The unbound fluorescence was removed by washing the cells 3× with medium, and cell-bound fluorescence was imaged using an Olympus confocal microscope. The experiment was done in triplicate.

### Binding Assay

Method 1: HT1080-FAP cells (200000 cells/well) were seeded in a 24-well plate. The cells were allowed to grow as a monolayer over 24 h and incubated with various concentrations of FAP-targeted rhodamine conjugate either in the presence or absence of excess competition ligand (ligand without dye). After incubating for 1 h at 4° C. the cells were washed 3× with PBS to remove unbound fluorescence. The cells were then dissolved in 1% SDS and the cell-bound fluorescence was measured using a Neo2 Plate Reader. The results are shown in FIG. 6.

Method 2: 100,000 HT1080-hFAP and HT1080 cells were seeded in amine-coated 24-well plates to ensure cell adherence. Upon formation of a monolayer, cells were incubated with various concentrations of the compound in the presence or absence of excess of unlabeled ligand. After incubation for 1 h, the cells were washed 3× with medium to remove to unbound fluorescence and dissolved in 1% SDS. The cell-bound fluorescence was measured using a fluorescence spectrophotometer (NeO2 Plate reader) set with a λₑₓ=555 nm and λₑₘ=575 nm. Cell-bound fluorescence was plotted against various concentrations and the apparent K_{d} was determined by using one-site binding (hyperbola) curve fit in GraphPad prism7. The experiment was done in triplicate.

### Ex Vivo Fluorescence Imaging and Biodistribution:

Female nu/nu athymic (5-6 weeks old) mice were subcutaneously injected with 5×10⁶ KB, MDA-MB231, HT29, U87MG, FaDu, PANC1 (with 20% matrigel) and BalbC mice for 4T1 cells in 0.1 mL sterile PBS. Tumors were allowed to grow to approximately 250-600 mm³ before initiating imaging studies. Each tumor-bearing mouse was intravenously injected (via tail vein) with 5 nmol to 10 nmol of the compound either in the presence or absence of a 10- to 500-fold excess of unlabeled ligand. Whole body images were acquired using an AMI instrument at two different time points (2 h and 6 h post-injection) for all the tumors followed by euthanasia using CO₂ asphyxiation. After performing whole-body imaging, organs of interest were harvested and imaged to quantitate fluorescence accumulation. The image acquisition parameters were as follows: i) lamp level-high; ii) excitation-745 nm; iii) emission-810; iv) binning (M) 4M; (v) f-stop-4; (vi) FOV-12.5; (vii) acquisition time, 5 s; (viii) power 55. Images were acquired at 2 h, 6 h, 8 h, 24 h, 32 h, 48 h, 72 h, 96 h, and/or 122 h.

### Western Blot Analysis:

After 24 hours of co-incubation, supernatant was removed, and cells were washed with phosphate-buffered saline (PBS). Cells were harvested and lysed for Western blot analysis. 10% sodium dodecyl sulphate polyacrylamide gel was used, and samples were analyzed via gel electrophoresis following by blocking. Nitrocellulose membranes were then incubated with antibodies to detect phosphorylated Akt\hat{ }Ser473 and alpha actin, and signals were visualized by Odyssey CLx imaging system (FIG. 23).

### Quantitative PCR

RNA was extracted using Quick-RNA Microprep Kit based on manufacturer's specification (Zymo Research; catalog number R1050). Extracted RNA was incubated with DNase I supplied from the kit for 15 minutes at room temperature. RNA underwent reverse transcription by high-capacity cDNA reverse transcription kit according to manufacturer's specification (Thermo Fisher; catalog number 4368814). cDNA was then mixed with Cyber-green supermix and primers for human collagen 1a1 and human alpha smooth muscle actin to perform quantitative PCR to analyze the expression of these two pro-fibrotic markers (FIG. 24).

1. A compound represented by a structure of formula (X):
Aₘ-L-B (X)
wherein
A is a radical of a fibroblast activation protein alpha (FAPα) ligand comprising a structure of formula (X-A): 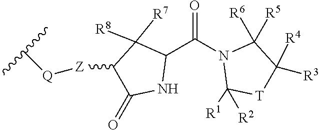
wherein:
Q is aryl, heteroaryl, or heterocyclyl;
Z is a bond, substituted or unsubstituted C₁-C₃ alkylene, substituted or unsubstituted heteroalkylene, amino, —O—, or —S—;
T is substituted or unsubstituted methylene, substituted or unsubstituted amino, —O—, or —S—;
R¹ and R² are each independently selected from the group consisting of —H, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, —SO₂F, —CONH₂, and 5-tetrazolyl;
R³ and R⁴ are each independently selected from the group consisting of —H, —OH, F, Cl, Br, I, —C₁₋₆alkyl, —O—C₁₋₆alkyl, and —S—C₁₋₆alkyl; and
R⁵, R⁶, R⁷, and R⁸ are each independently selected from group consisting of H, alkyl, and halo;
L is a linker connecting one or more A groups to B;
B is a radical of an optical dye, a photodynamic therapeutic agent, a radio-imaging agent, a radiotherapeutic agent, a chemotherapeutic agent, an antifibrotic agent, or an anticancer agent; and
m is 1-6.

2-39. (canceled)

40. The compound of claim 1, wherein A comprises a structure of formula (X-B): 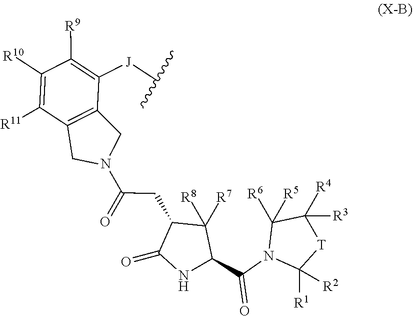
wherein
Q is aryl, heteroaryl, or heterocyclyl;
T is substituted or unsubstituted methylene, substituted or unsubstituted amino, —O—, or —S—;
J is C(R^{J})₂, wherein each R is independently H or alkyl, or both R are taken together to form oxo;
R¹ and R² are each independently selected from the group consisting of —H, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, —SO₂F, —CONH₂, and 5-tetrazolyl;
R³ and R⁴ are each independently selected from the group consisting of—of —H, —OH, F, Cl, Br, I, —C₁₋₆alkyl, —O—C₁₋₆alkyl, and —S—C₁₋₆alkyl;
R⁵, R⁶, R⁷, and R⁸ are each independently selected from group consisting of H, alkyl, and halo; and
R⁹, R¹⁰, and R¹¹ are each independently selected from group consisting of H, —C₁₋₆alkyl, —C₁₋₆haloalkyl, —O—C₁₋₆alkyl, —S—C₁₋₆ alkyl, F, Cl, Br and I.

41. The compound of claim 1, wherein A is selected from the group consisting of:

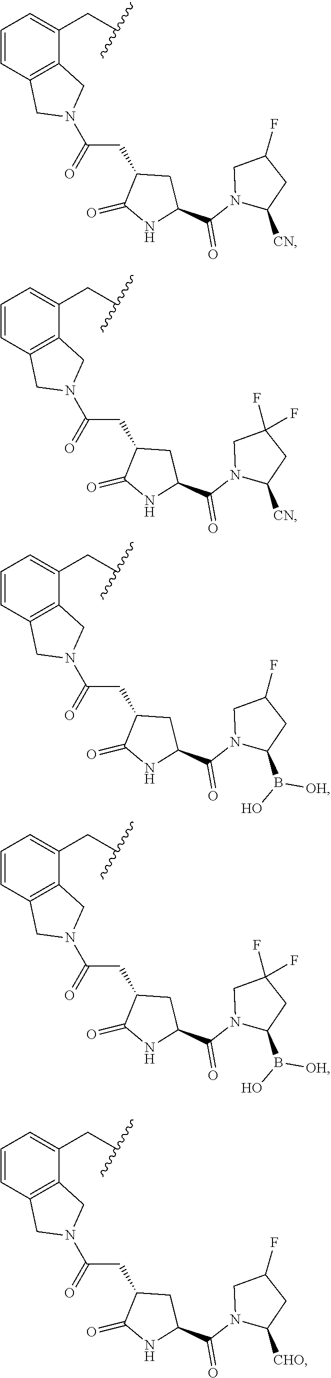 [SMILES: *Cc1cccc2c1CN(C(=O)C[C@@H]1C[C@@H](C(=O)N3CC(F)C[C@H]3C#N)NC1=O)C2 ; *Cc1cccc2c1CN(C(=O)C[C@@H]1C[C@@H](C(=O)N3CC(F)(F)C[C@H]3C#N)NC1=O)C2 ; *Cc1cccc2c1CN(C(=O)C[C@@H]1C[C@@H](C(=O)N3CC(F)C[C@H]3B(O)O)NC1=O)C2 ; *Cc1cccc2c1CN(C(=O)C[C@@H]1C[C@@H](C(=O)N3CC(F)(F)C[C@H]3B(O)O)NC1=O)C2 ; *Cc1cccc2c1CN(C(=O)C[C@@H]1C[C@@H](C(=O)N3CC(F)C[C@H]3C=O)NC1=O)C2]

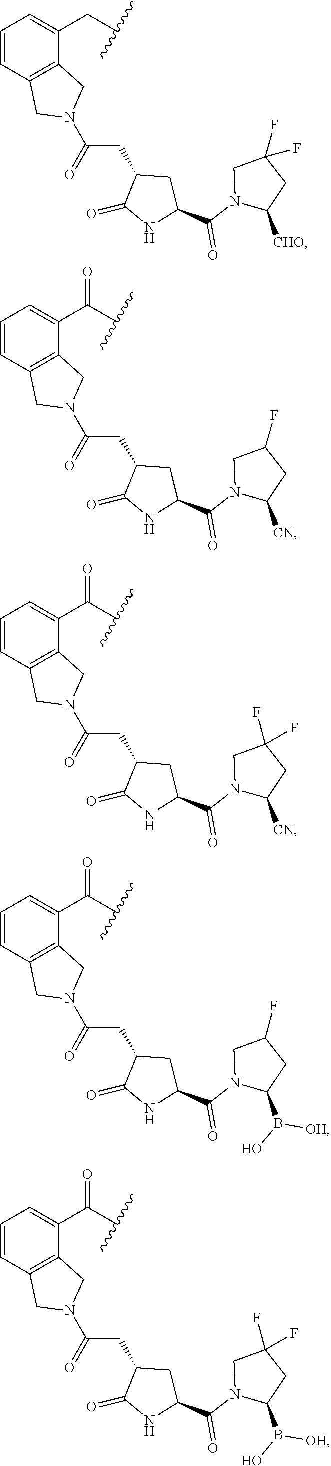 [SMILES: *Cc1cccc2c1CN(C(=O)C[C@@H]1C[C@@H](C(=O)N3CC(F)(F)C[C@H]3C=O)NC1=O)C2 ; *C(=O)c1cccc2c1CN(C(=O)C[C@@H]1C[C@@H](C(=O)N3CC(F)C[C@H]3C#N)NC1=O)C2 ; *C(=O)c1cccc2c1CN(C(=O)C[C@@H]1C[C@@H](C(=O)N3CC(F)(F)C[C@H]3C#N)NC1=O)C2 ; *C(=O)c1cccc2c1CN(C(=O)C[C@@H]1C[C@@H](C(=O)N3CC(F)C[C@H]3B(O)O)NC1=O)C2 ; *C(=O)c1cccc2c1CN(C(=O)C[C@@H]1C[C@@H](C(=O)N3CC(F)(F)C[C@H]3B(O)O)NC1=O)C2]

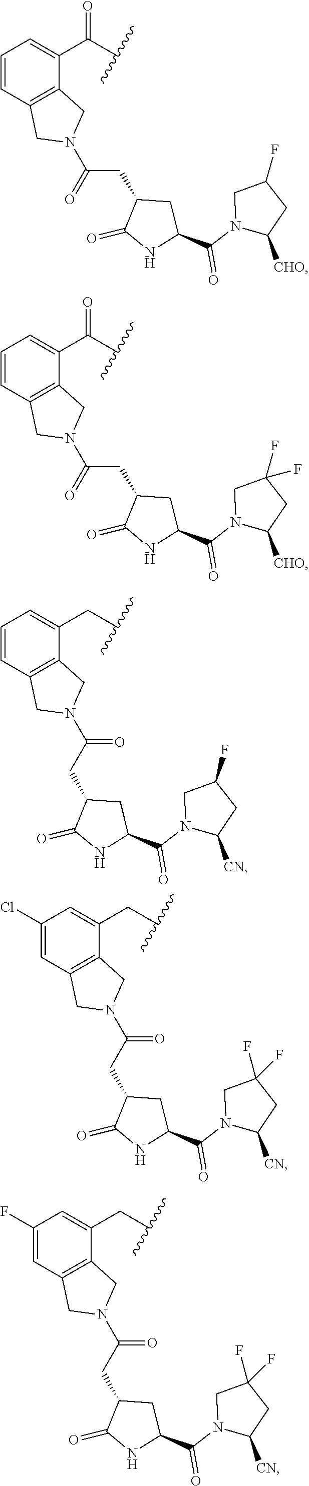

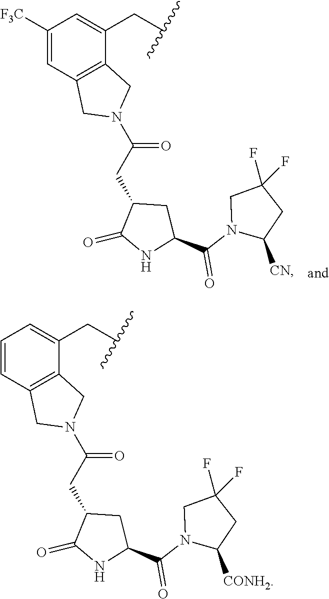 [SMILES: *Cc1cc(C(F)(F)F)cc2c1CN(C(=O)C[C@@H]1C[C@@H](C(=O)N3CC(F)(F)C[C@H]3C#N)NC1=O)C2 ; *Cc1cccc2c1CN(C(=O)C[C@@H]1C[C@@H](C(=O)N3CC(F)(F)C[C@H]3C(N)=O)NC1=O)C2]

42. The compound of claim 1, wherein:
L is (L¹)ₒ-Y-(L₂)ₚ, wherein:
each L¹ is a first linker;
each L² is a second linker;
Y is a third linker;
is an integer from 1-5; and
p is an integer from 1-5.

43. The compound of claim 42, wherein each L¹ and L² independently comprises one or more linker group, each linker group independently selected from the group consisting of alkyl(ene), heteroalkyl(ene), heterocycloalkyl(ene), heteroaryl, aryl, alkoxy, thioether, disulfide, carboxylic acid, anhydride, carbonate, carbamate, thioether, sugar, and peptide.

44. The compound of claim 42, wherein each L¹ and L² independently comprises one or more linker group, each linker group independently selected from the group consisting of polyethylene glycol (PEG), alkyl(ene), disulfide, amide, carboxylic acid, anhydride, carbonate, ester, carbamate, thioether, phenyl, and triazole.

45. The compound of claim 42, wherein Y has an amine core, an aromatic core, or an alkylene core.

46. The compound of claim 42, wherein L, L¹, L², or any combination thereof independently comprise at least one linker group having the following structure: 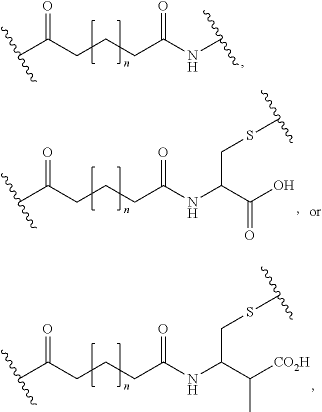
wherein n is 0 to 10.

47. The compound of claim 42, wherein L, L¹, L², or any combination thereof independently comprises at least one linker group having the following structure:

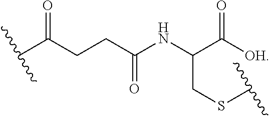 [SMILES: *SCC(NC(=O)CCC(*)=O)C(=O)O]

48. The compound of claim 1, wherein L, L¹, L², or any combination thereof comprises at least one linker group having the following structure: 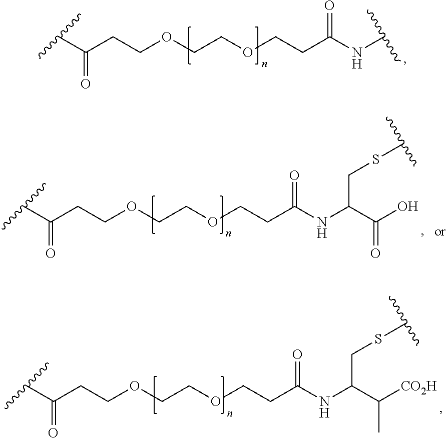
wherein n is 1 to 32.

49. The compound of claim 1, wherein L has the following structure:

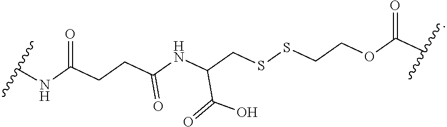 [SMILES: *NC(=O)CCC(=O)NC(CSSCCOC(*)=O)C(=O)O]

50. The compound of claim 1, wherein the compound is an imaging agent and B is a radical of a fluorescent dye.

51. The compound of claim 1, wherein the compound is a chemotherapeutic agent and B is a radical of an antifibrotic agent or an anticancer agent.

52. The compound of claim 1, wherein B is radical of a phosphoinositide 3-kinase (PI3K) inhibitor.

53. The compound of claim 1, wherein B has a structure of a radical of a compound represented by the following formula: 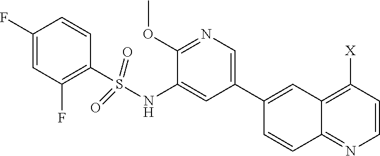
wherein:
X is selected from the group consisting of

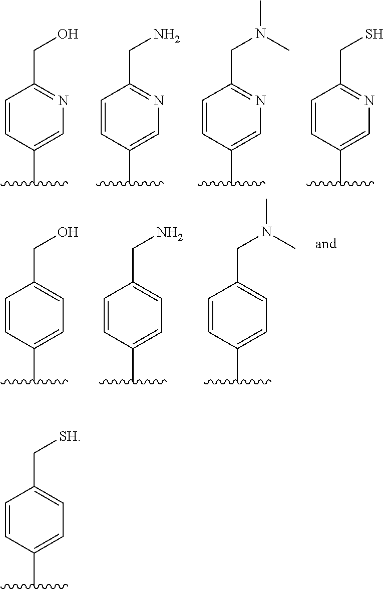

54. The compound of claim 1, wherein B is a radical of a compound of the following structure:

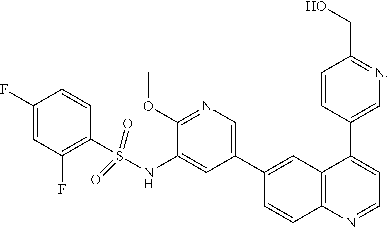

55. A compound of the following structure:

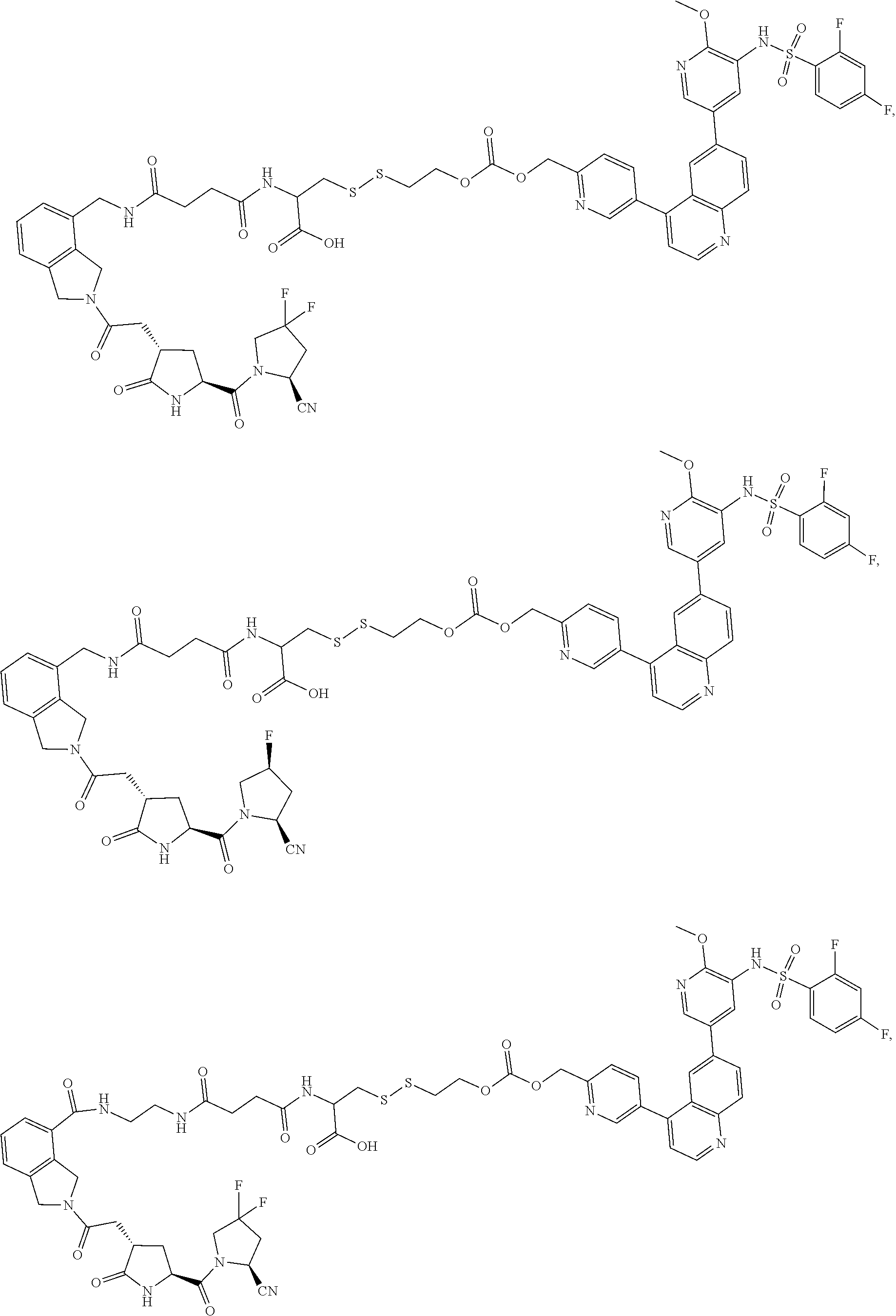

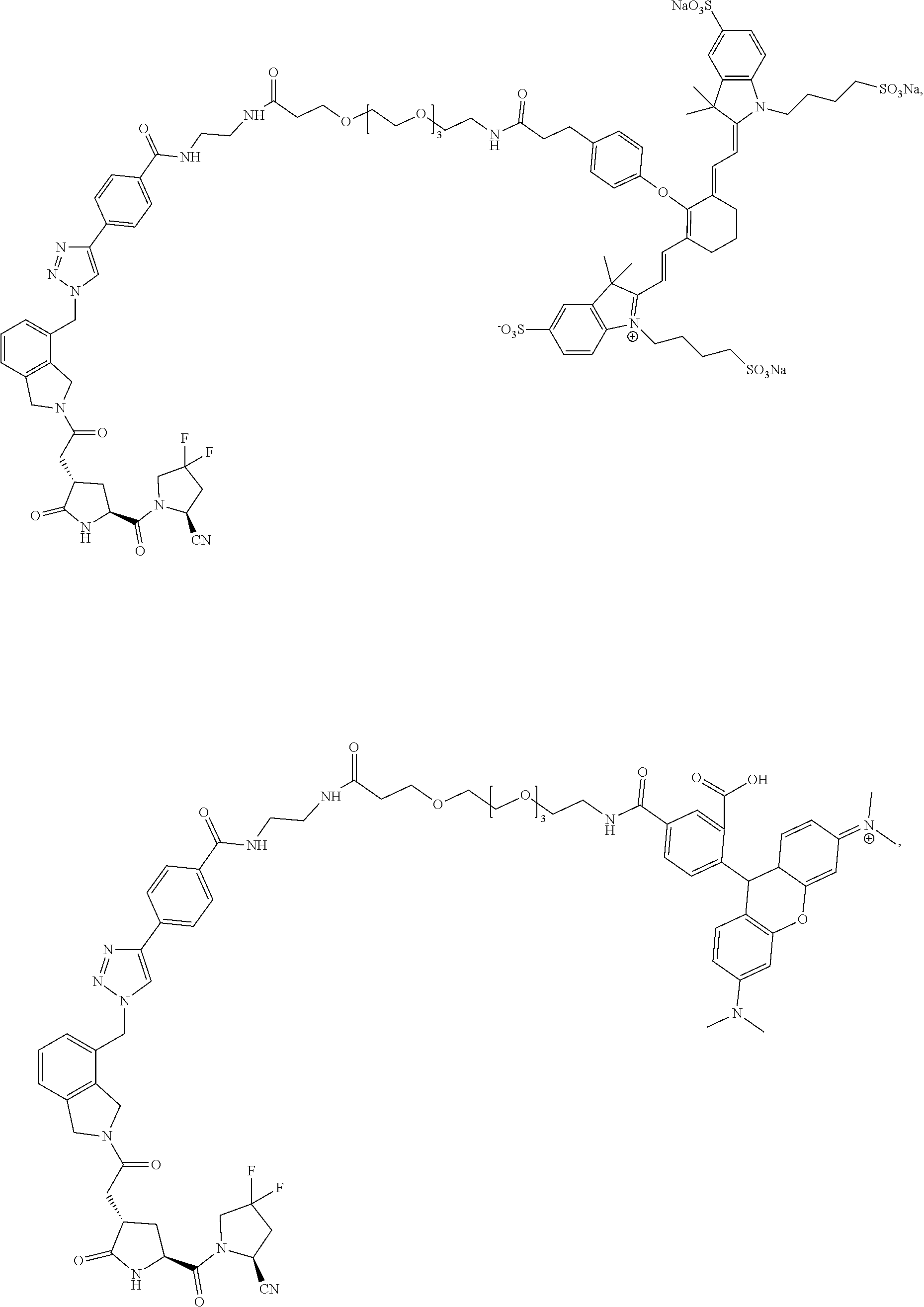

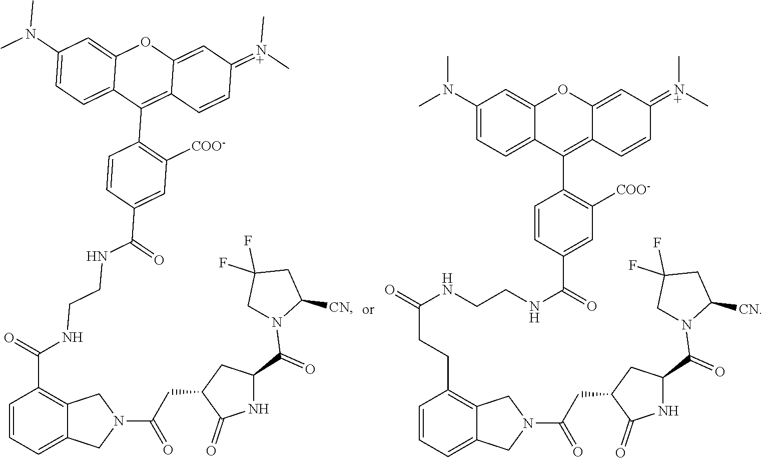

56. A pharmaceutical composition comprising a compound of claim 1 and a pharmaceutically acceptable carrier.

57. A method for imaging cancer or fibrosis in a subject with the cancer or the fibrosis, the method comprising administering an effective amount of a compound of claim 1 to a subject in need thereof.

58. A method for treating an inflammatory disease or disorder, the method comprising administering a therapeutically effective amount of a compound of claim 1 to a subject in need thereof.

59. A method for treating cancer, the method comprising administering a therapeutically effective amount of a compound of claim 1 to a subject in need thereof.

60. The method of claim 59, wherein the cancer is selected from the group consisting of fibrosarcoma, breast cancer, colorectal cancer, glioblastoma, squamous cell carcinoma, pancreatic cancer, and cervical cancer.

61. A method for treating fibrosis in a subject in need thereof, the method comprising administering a therapeutically effective amount of a compound of claim 1 to the subject.

62. The method of claim 61, wherein A comprises:

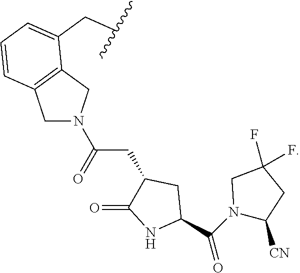

63. The method of claim 57, wherein A comprises:

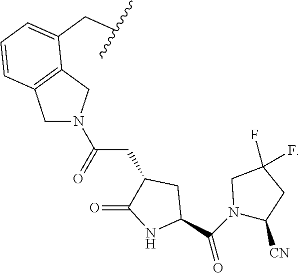

64. The method of claim 58, wherein A comprises:

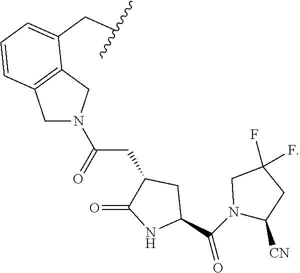

65. The method of claim 59, wherein A comprises:

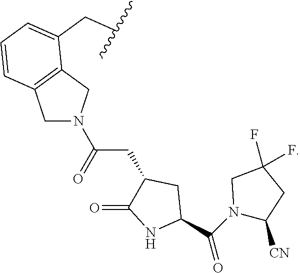

66. A conjugate represented by a structure of formula:
FAP-A-L-B
wherein
FAP is fibroblast activation protein alpha (FAPα), bound to the A group;
A is a radical of a fibroblast activation protein alpha (FAPα) ligand comprising a structure of formula (X-A): 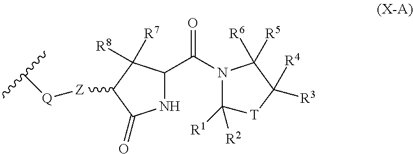
wherein:
Q is aryl, heteroaryl, or heterocyclyl;
Z is a bond, substituted or unsubstituted C₁-C₃ alkylene, substituted or unsubstituted heteroalkylene, amino, —O—, or —S—;
T is substituted or unsubstituted methylene, substituted or unsubstituted amino, —O—, or —S—;
R¹ and R² are each independently selected from the group consisting of —H, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, —SO₂F, —CONH₂, and 5-tetrazolyl;
R³ and R⁴ are each independently selected from the group consisting of —H, —OH, F, Cl, Br, I, —C₁₋₆alkyl, —O—C₁₋₆alkyl, and —S—C₁₋₆alkyl; and
R⁵, R⁶, R⁷, and R⁸ are each independently selected from group consisting of H, alkyl, and halo;
L is a linker connecting one or more A groups to B; and
B is a radical of an optical dye, a photodynamic therapeutic agent, a radio-imaging agent, a radiotherapeutic agent, a chemotherapeutic agent, an antifibrotic agent, or an anticancer agent.

67. The compound of claim 66, wherein FAP is expressed on the surface of a fibroblast.

68. The compound of claim 1, wherein A comprises

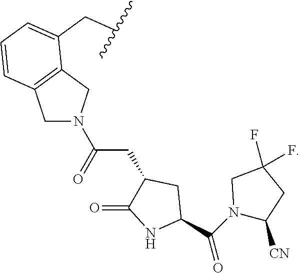

69. The pharmaceutical composition of claim 56, wherein A comprises:

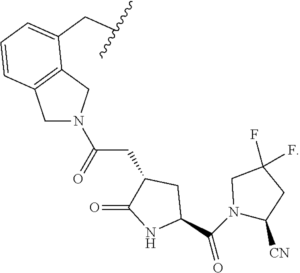

70. A compound of the following structure: 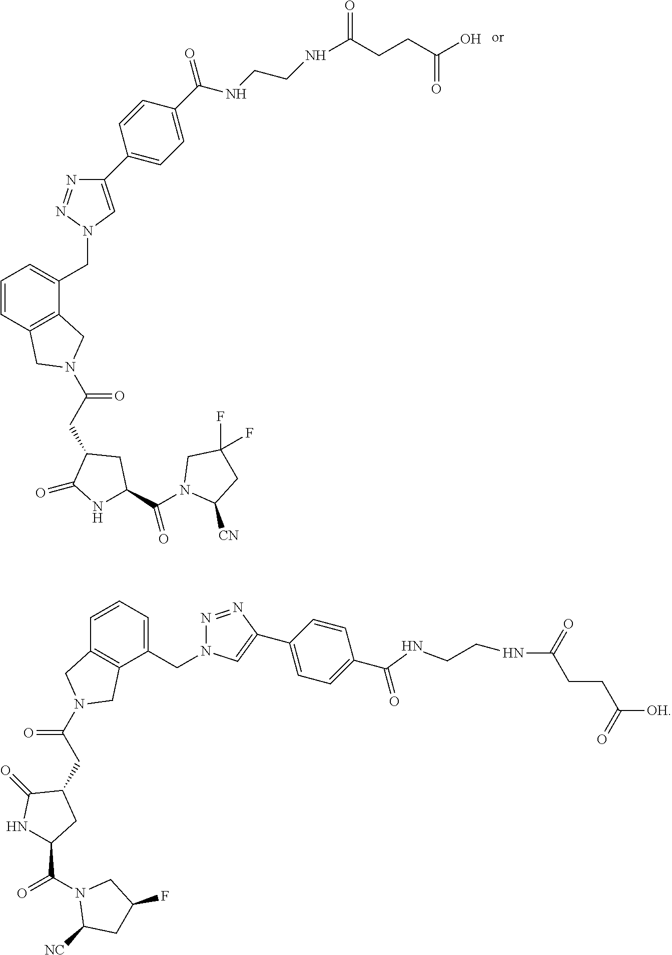 [SMILES: N#C[C@@H]1CC(F)(F)CN1C(=O)[C@@H]1C[C@@H](CC(=O)N2Cc3cccc(Cn4cc(-c5ccc(C(=O)NCCNC(=O)CCC(=O)O)cc5)nn4)c3C2)C(=O)N1 ; N#C[C@@H]1C[C@H](F)CN1C(=O)[C@@H]1C[C@@H](CC(=O)N2Cc3cccc(Cn4cc(-c5ccc(C(=O)NCCNC(=O)CCC(=O)O)cc5)nn4)c3C2)C(=O)N1]
